SEQUENCE LISTING (88 sequences)

SEQ ID NO: 1 - DNA, 1668 bp, Physcomitrella patens
  CDS (242)..(1588): CBL-interacting protein kinase (EST462)
atcccgggtgtaaggtggaggaatggcactgtgacacacggctgatttttgaagaaacgagctccgggtgaaaaatgaaaatgagttgcggtgcaggatgtggaagcgttcgtcagacagcatgagaagatttgtgtgcccagactctttttattgtatgttagggaaggaaagatatcgcgaaaccagcgcaagactgagaagggtgaaagttagataggttacttacgtacaagcaaacatgactaccgcgacaccaagtatcccggctacgaacgtggagcgcacgcgggtcggcaaatatgatctcggcaagaccctgggagagggcacatttgccaaagtcaaggtggctaagcacatcgacactggccatactgttgccataaagattttggacaaggacaagattctcaagcataagatggttgagcagatcaaaagagaaatatctaccatgaagctagtgaagcacccttacgtcgtccagctgttggaagttatggccagcaggacaaaaatctatattgtgctggagtatgttacaggtggcgaacttttcaacaagattgctcaacaaggaaggctgtcagaggacgacgcaaggaaatactttcagcagctcattgatgcagttgattattgccacagccggcaagtttttcatagagatttgaagccagagaatctccttctggatgcgaaggggagcttgaaaatttcggactttggtttgagtgcgctaccgcagcaatttagggctgatggattattacacacaacttgcggaacacccaattatgtggctcctgaggtgattatggataagggatattcgggcgctactgctgatttgtggtcttgcggtgtcatcttatacgtgctgatggctgggtacttgccttttgaggagcccactattatggctttgtacaagaagatatatcgggctcaattctcatggcctccctggttcccgtcaggtgcccggaaattaatttcaaagatattggatcccaaccctagaactcgcatctcagcagctgaaatttataaaaatgattggttcaagaagggatacactccagctcagtttgaccgagaagctgatgtcaaccttgatgatgtgaatgctatcttcagcggttcacaagaacatatagttgtagaaaggaaggaatcaaaaccggttactatgaacgcttttgagctcatctctttgtcttcgggcctcaatctttctagtttgtttgagacaaaagagattcctgaaaaggaggacacgcggtttacaagcaagaaatctgccaaagagatcatcagttcaatcgaggaagctgcgaagcccttgggctttaatgttcagaagcgagattataagatgaagttacaaggagacaagctgggcaggaagggacatctttcagtctcaaccgaggtgttcgaggtggcgccttctctttacatggttgagttacagaagaacagtggtgatacattggagtataaccatttttacaagaatctttccaagggcctaaaagacatagtgtggaaagcagaccctcttcctgcatgtgaacaaaagtagacgcttccgctacggcttcaaaataagcccgtgccgtgaagtacccacatctcctcacttggcatctcagttaacgc

SEQ ID NO: 2 - protein, 449 aa, Physcomitrella patens
MTTATPSIPATNVERTRVGKYDLGKTLGEGTFAKVKVAKHIDTGHTVAIKILDKDKILKHKMVEQIKREISTMKLVKHPYVVQLLEVMASRTKIYIVLEYVTGGELFNKIAQQGRLSEDDARKYFQQLIDAVDYCHSRQVFHRDLKPENLLLDAKGSLKISDFGLSALPQQFRADGLLHTTCGTPNYVAPEVIMDKGYSGATADLWSCGVILYVLMAGYLPFEEPTIMALYKKIYRAQFSWPPWFPSGARKLISKILDPNPRTRISAAEIYKNDWFKKGYTPAQFDREADVNLDDVNAIFSGSQEHIVVERKESKPVTMNAFELISLSSGLNLSSLFETKEIPEKEDTRFTSKKSAKEIISSIEEAAKPLGFNVQKRDYKMKLQGDKLGRKGHLSVSTEVFEVAPSLYMVELQKNSGDTLEYNHFYKNLSKGLKDIVWKADPLPACEQK

SEQ ID NO: 3 - DNA, 1115 bp, Physcomitrella patens
  CDS (46)..(816): 14-3-3 protein (EST362)
atcccgggctcgctcgcttgggtgcagtaacgaccgagatcgaccatggcgacggaggcgcgcgaggagaatgtgtacatggccaagctggccgagcaggccgagcgctacgacgagatggtggaggccatggagaaggtggccaagaccgtcgacaccgaggagctcaccgtcgaagagcgcaacttgttgtctgtggcttacaagaacgtgattggcgctcggagggcgtcgtggaggatcatctcctccatcgagcagaaggaggagagcaagggaaacgacgagcacgtttccgccatcaaggagtaccgtggcaaggtggagtctgagttgagcaccatctgtgacagtattcttaagcttctggatacccacctgatccctacttctagctctggggagtcgaaagttttctacttgaagatgaagggtgattatcacaggtacttggctgagtttaagaccggggccgagaggaaggaagctgctgaagcgacattgttggcgtataagtctgctcaagatattgcgttgacagagttggctcctacccaccccatcagactgggtttggcattgaacttctctgtgttttattacgagattcttaactcaccagatcgggcgtgcactcttgcgaagcaggcatttgatgaagcgatcgctgagcttgatactcttggagaggagtcttacaaggatagcactcttattatgcagctcctccgcgacaacctgacgttgtggacctctgatatgcaggatgaggtcggccccgaggtcaaggatgccaaagttgatgatgctgagcactgaagtggaacttaagctatatttatctttgcacagcagagaggtcatggttagtggatgattttcccgctcggtgcgagtagtggtgcaataccagagacttttctattgccggatcaggacattgtgggacttttctggcaagtccgtggagaagccgctgctttgcgaagcacttctgttgtggttaatttacaggttggtgcttgtgcttttccagttgctcttatagtgccggtatctttgtaagcaagcgagttgtttatttgtctggtggatgacgcatcttccgatatcgc

SEQ ID NO: 4 - protein, 257 aa, Physcomitrella patens
MATEAREENVYMAKLAEQAERYDEMVEAMEKVAKTVDTEELTVEERNLLSVAYKNVIGARRASWRIISSIEQKEESKGNDEHVSAIKEYRGKVESELSTICDSILKLLDTHLIPTSSSGESKVFYLKMKGDYHRYLAEFKTGAERKEAAEATLLAYKSAQDIALTELAPTHPIRLGLALNFSVFYYEILNSPDRACTLAKQAFDEAIAELDTLGEESYKDSTLIMQLLRDNLTLWTSDMQDEVGPEVKDAKVDDAEH

SEQ ID NO: 5 - DNA, 2601 bp, Physcomitrella patens
  CDS (1091)..(2233): RING H2 zinc finger protein (EST373)
atcccgggcgtgtgagtaccctcattgctcgcagcagcatcatcaggttgtactgctcgaagcgaacgtttattgaatggccaccacaattgatcttgatgtgtgggtggacggttgcaataaactcttttagcagcgctagatggcgttttcttaggccaagctgagagtcataagcgagtcagtttttgggtgaccatcactgcttatcgattcgtgagaagcattccacttggaattgcggatggttagtcaaggatagtgaattggatgatgtagatgatttttacccacacatgggctgctgctcggtctgcagttcggtcctatgcagcatcaggatgatgcttttgcttctgccaggacttcaccgggtcataacgagtccggagaggtacaaccgagggttagatgttggtgagcatggttgggcgagttgacacccttgtcctcaattcatccgtcgttttcgcaatctgctgttcctagttctgcatgcaagcttccgtttcgagagtgtgagtgacaactgttctagatccctaaaggatcagatattcgggaactcaagggtgctgttgcaattttcgaaagatgtggatggggtacaaccacgcgctagtgcgaggagcgacaagcaaaccgatgaggggaagcggagctcttgcagtcactgttcgtattagaattgaggattttagcaacagaaggtcttgtggatctaagtccctgcgtttggcgatggaagttggtctcatcagctgaaatcctttgtagtcgctaaacggccgagtttagtgtctggcggaattgaccattctgcagcactccaaggtctttcagctgatatgaaacaattgacaaatgaggtatgcaaatactgtgggttgcgagacaagttcacaagacatttgattcaggatatataaccccatgcatagattatccaagcgtcacttagcagggatatttcagttttagaacagaatttgctaattgggcgaagctcttcaagttgatagtttcatgaatttccactcattactggagtctgcgccagtttttcgaagtatcaaggagagtggtcaaaatggcggcgttgatggttgagacgcccatagccttcgggcttacgatggcggtgtgtttggctttattcttctattgttggcgcattcggaagtttcgtaatcggctcacctccgtccaagtcgcagccacgcctaatgaagtgaattcagggttgcagattggaatcaagcaggatgtgatcaaaaccttcccaactgtgatgactaaggagctgaaaattgacatcaaggatgggcttcagtgcccgatatgtctggtcgagtacgaggaggcggaagtgctgcgaaaacttccactctgcggccatgttttccacatacgttgcgtcgactcctggctagaaaagcaagtcacttgtcctgtttgccgcattgttctcgcgggagtttccaagttatcacttcgaactaaccgccagcaaaactatcttaatcactacagatttccctccagcccccgctctgtaaccgtagaggtggctggcaacatacccgcatgggttcttgtcaatcgacctctgcccttgccaccagccattcctgagcgcccctcggtggacagcgtcacctctctagaatccagccccttggacattgatgtgcagccttcagccaatttcggcatgaccggcgagtctccactcctcattcctcacgatgcaggatggggagctatctacctgcagaggagtcatggcgcactgagctttaaggcgcgaacaggcgcagacatcgcaatcgaaaccaaagagtgcgtcgatcattcttccataagcgagaggtggatgacagagtcgttctcttttggcatctccacctgcgaggacgtgtcttcgacaagatctagccataatgtgtggcaagctgactcgactacacgccattcttcgtggagctcacactcccacaactcattgtgtgatatcaaccaacccacgatgaagaattgggagtcggaggaagtgtttgagtcgctagccacccatcaccagcccttgacgatgtccccagagcgctgctcctttgagtttctgcccatcatcacaggcactgaaggtgactgcattttgaagcacaattcttatgcgccgaaaccagaaagaactgagatcggttcaagccctcactcttactcccagctctgaatttttcctcccgaattctggagaaccatctcttcaccacattagtgcactccgcaaatttcttcatggtcatgactgttggaagcattcatttttcgggagggcggagtgcaccgctggttttacgtgtctcgcaacgaaggtttagaaggggactgtcggagaagattggtttgctcgaaaagagttgctccgttgaagaagcacttttacgggacggaatcccaaaccgaaaataaggttcaaattttaggcagagtagatggtaacaaactgtacattcacactgtggcttaaggaatcaccgccggaatgtagtaatcttgtaaataatcacccagccgtgatcttagaggcgttaacgc

SEQ ID NO: 6 - protein, 381 aa, Physcomitrella patens
MAALMVETPIAFGLTMAVCLALFFYCWRIRKFRNRLTSVQVAATPNEVNSGLQIGIKQDVIKTFPTVMTKELKIDIKDGLQCPICLVEYEEAEVLRKLPLCGHVFHIRCVDSWLEKQVTCPVCRIVLAGVSKLSLRTNRQQNYLNHYRFPSSPRSVTVEVAGNIPAWVLVNRPLPLPPAIPERPSVDSVTSLESSPLDIDVQPSANFGMTGESPLLIPHDAGWGAIYLQRSHGALSFKARTGADIAIETKECVDHSSISERWMTESFSFGISTCEDVSSTRSSHNVWQADSTTRHSSWSSHSHNSLCDINQPTMKNWESEEVFESLATHHQPLTMSPERCSFEFLPIITGTEGDCILKHNSYAPKPERTEIGSSPHSYSQL

SEQ ID NO: 7 - DNA, 874 bp, Hordeum vulgare
  CDS (15)..(611): RING H2 zinc finger protein (HV62561245)
gcgagggggaaacgatgatgttcgggtcggggatgaatctcctcagcgcggcgctcggcttcggcatgaccgccgtcttcgtcgcgttcgtctgcgcgcggttcatctgctgccgcgcccggggcgcgggcgacggcgccccgccgccggtggactttgacgttgacttcccggcagatctcgaacgcccggtggaggatgctcattgtgggttggagcctttggttattgctgcaattcctattatgaagtactccgaggaattatattcaaaggatgatgcccagtgctccatatgtctaagtgaatacactgagaaagagcttctaagaatcattccgacatgtcggcataactttcaccgttcctgcttagatttatggttgcagaaacagactacttgcccaatatgccgggtctcgttaaaagagctgcctagcagaaaagctgctataacaccttcatgtagcaaccctcaagtgtgccctcgcactgagaactctgttaatccagcacctgactggctcctccctgttcatcattctcacagaggtcaacaaagtggtttagacacacaaggatcagtagaagtgattattgagatacgccaataagcacagcatgaggttgctatggaagagagcaaaatgggaatatgtaataggtttcctgcctcattgcattgttgcagcaccctaactggattggcattgtatgccacctcgttgcaggtaatgtgtaaacatttgttgtacatttcacattgtagataagcatattgtgttatgacacataaatactttcaatgttcttttctaatgcactgtatattgtaaaaatggtaaggaaatattggatgttagataaattcctg

SEQ ID NO: 8 - protein, 199 aa, Hordeum vulgare
MMFGSGMNLLSAALGFGMTAVFVAFVCARFICCRARGAGDGAPPPVDFDVDFPADLERPVEDAHCGLEPLVIAAIPIMKYSEELYSKDDAQCSICLSEYTEKELLRIIPTCRHNFHRSCLDLWLQKQTTCPICRVSLKELPSRKAAITPSCSNPQVCPRTENSVNPAPDWLLPVHHSHRGQQSGLDTQGSVEVIIEIRQ

SEQ ID NO: 9 - DNA, 1153 bp, Brassica napus
  CDS (44)..(847): RING H2 zinc finger protein (BN43173847)
ctctctccctctcaatctctcattcgccaccatcttcaaactcatgaactccaacgaccaatatccaatgggcaggcccgacgaaaccacctccggctcttctcgaacctacgccatgagcgggaagatcatgctgagcgccatcgtcatcctcttcttcgtcgtcatcctaatggtcttcctccacctctacgcccgctggtacctcctccgcgctcgccgccgtcatttccgccgccgcagccgtaaccgtcgctccacgatggttttcttcgccgcggatccttccgccgccgccgccgcctcgcgcggcctcgatcccgcggtgatcaagtctctccccgttttcgctttctccgagttgactcacaaagatctgaccgagtgcgccgtttgcctctccgagttcgaggaaggcgagtcgggtcgggttttgcccggttgcaagcatacgtttcatgttgactgtatagatatgtggtttcattctcattccacgtgtcctctctgccgctctctcgtcgagcctcccgtggaggagcaagttgcgatcacgatttctcctgaaccggtttctgttgcaattgaaccgggttcgagctctggattgagaaaaccggcggcgattgaggtgccgaggaggaacttcagtgaatttgacgatcggaactcgccggcgaatcactcgtttaggtcgccgatgagtcgtatgttatctttcactcggatgctgagcagaggaaactcctcgtcgcccatagccggagctccgcctcaatctccgtcgtctaactgccggatagcgatgactgagtcagatatagagcgtggaggagaagagactaggtgagctattggtcggaaagtaaaaactataaattttattacaggattgataaagtcaactagcctttgccgacggttgatttaagctccagtaacacgttgcgtggtctgaacgaatcttattcaccgagtgtttacttgtgttagtttagatagaattgtctgaagatgtacataaaattgtcagttgtcgatgatgttatattgaatcttttttttccatttgtttttattcccagtctctatagactctttatgtaataccaccaattcaatggtcatgaaatcatgatagagacttaacctg

SEQ ID NO: 10 - protein, 268 aa, Brassica napus
MNSNDQYPMGRPDETTSGSSRTYAMSGKIMLSAIVILFFVVILMVFLHLYARWYLLRARRRHFRRRSRNRRSTMVFFAADPSAAAAASRGLDPAVIKSLPVFAFSELTHKDLTECAVCLSEFEEGESGRVLPGCKHTFHVDCIDMWFHSHSTCPLCRSLVEPPVEEQVAITISPEPVSVAIEPGSSSGLRKPAAIEVPRRNFSEFDDRNSPANHSFRSPMSRMLSFTRMLSRGNSSSPIAGAPPQSPSSNCRIAMTESDIERGGEETR

SEQ ID NO: 11 - DNA, 1069 bp, Brassica napus
  CDS (48)..(881): RING H2 zinc finger protein (BN46735603)
tttcacccaactctctctctctcagttcccactcgtgatccgaaagcatgagtcttagagacccgaatccagtaactaacacacccggatccttttcggatccaggcgggttcgctataaacagcagaatcatgttcaccgccataatcataatcatattcttcgtcattctcatggtctctcttcacctctactctcgttgctacctccaccgctctcgccgtttccacatccgccgcttaaaccgtagtagacgcgccgccgccgctatgaccttcttcgccgatccttcctcctccacctccgaggtcaccactcgcggtctcgacccctccgtcgtcaaatctcttcccactttcacgttctccgccgcagccgccccggacgcgatcgagtgtgcggtttgcctctcggagtttgaggagagcgaaccgggtcgggttttgcccaattgcaagcacgcgtttcatgttgagtgcattgatatgtggtttctttctcattcctcttgtcctctgtgccgatcgctcgtcgaacctatcgccggagttgtaaaaactgcggcggaggaagtcgcgatttcgatttctgacccggtttcaggcgacacaaacgacgttataggagctgggacttccgatcatgaagattccagggggaaaccggcggcgattgaagtctcaacgaggaatctcggagaatcggagaacgagttgagtcggagtaactcgtttaagtcacgggtgatatcttccacgcggattttcagcaaagaacggagaagcgcttcgtcgtcttcttctatcgggttccctccgcctccggtctctagcatgccgatgacggagttagatatcgagtctggaggagaagagcctcgttgactttaagacgctaaatttttactgctacgtggacgtgtatgatttgttataaatgtttccttgtttagagctaagatgcggagatgaaataattctttgttagggcatcagcattgggacttcttaagcccatttcttagtaaatttgggtcgaaatttaaatcaaaaaggctggatatgtttgg

SEQ ID NO: 12 - protein, 278 aa, Brassica napus
MSLRDPNPVTNTPGSFSDPGGFAINSRIMFTAIIIIIFFVILMVSLHLYSRCYLHRSRRFHIRRLNRSRRAAAAMTFFADPSSSTSEVTTRGLDPSVVKSLPTFTFSAAAAPDAIECAVCLSEFEESEPGRVLPNCKHAFHVECIDMWFLSHSSCPLCRSLVEPIAGVVKTAAEEVAISISDPVSGDTNDVIGAGTSDHEDSRGKPAAIEVSTRNLGESENELSRSNSFKSRVISSTRIFSKERRSASSSSSIGFPPPPVSSMPMTELDIESGGEEPR

SEQ ID NO: 13 - DNA, 1214 bp, Glycine max
  CDS (49)..(1008): RING H2 zinc finger protein (GM52504443)
cctgccaccaaccaaaaccaatcctattacaacaagttcagcccttccatggccatcataatcgtcatcctcatcgccgccctctttctaatgggcttcttctccatctacatccgccactgctccgactccccctccgccagcatccgcaatctcgccgccgccactggacgctcacggcgcggcacccgcggcctcgagcaggcggtgatcgacaccttcccgacgctggagtactcggcggtgaagatccacaagctgggaaagggaactctggagtgcgctgtgtgcttgaacgagttcgaggacaccgaaacgctgcgtttaatccccaagtgtgaccacgtgttccaccccgagtgcatcgacgagtggctagcttcccacaccacttgccccgtttgccgcgccaacctcgtccctcagcccggcgagtccgtccacggaatcccaatcctcaacgctcctgaggacatcgaggcccaacacgaagcccaaaacgacctcgtcgagcccgaacagcaacagcaagaccccaagcctcccgttcccactgaacctcaagtgctgtcattaaaccagacgctgaaccggaaccgcaccagaggctcccggtcgggccggccgcggcgattcccgcggtctcactcgaccggtcattctttagtcctgccgggcgaagacactgaacggttcactttgcggcttcccgaggaagttagaaagcagatattgcagaacccgcaactgcatcgcgcgagaagcctcgttatcttaccgagagaaggtagttcgcggcgggggtatcgaaccggtgaaggaagtagcagagggagatcgtcgaggcggttggaccgggggtttaagtcggaccggtgggttttcaccatggcgccgccttttttggtgagagcgtcgtcgattaggtcgcccagggtggccaataacggtggcgaaggaacttccgctgctgcgtctttgcctccgccgcctgctgtggagtctgtttgagttttgattcccccttctgcaagatttcaatattttattgtatttaccaattattttttgctgccacgatttttttacgctagaatttgtaagatgtgtataatatttggcacacttgttttgcgtttgaagataaataactgaaatcctgaatcacgatagattcttaaatcataatcttggtcatcagttcagatatgaat

SEQ ID NO: 14 - protein, 320 aa, Glycine max
MAIIIVILIAALFLMGFFSIYIRHCSDSPSASIRNLAAATGRSRRGTRGLEQAVIDTFPTLEYSAVKIHKLGKGTLECAVCLNEFEDTETLRLIPKCDHVFHPECIDEWLASHTTCPVCRANLVPQPGESVHGIPILNAPEDIEAQHEAQNDLVEPEQQQQDPKPPVPTEPQVLSLNQTLNRNRTRGSRSGRPRRFPRSHSTGHSLVLPGEDTERFTLRLPEEVRKQILQNPQLHRARSLVILPREGSSRRGYRTGEGSSRGRSSRRLDRGFKSDRWVFTMAPPFLVRASSIRSPRVANNGGEGTSAAASLPPPPAVESV

SEQ ID NO: 15 - DNA, 981 bp, Glycine max
  CDS (4)..(660): RING H2 zinc finger protein (GM47122590)
gtgatgtctgagtgtggctgttccgagtcagacccttcgtgtggttgttggtcgagcagcagcagcagatctgtggcctcaactgaactgaagctgtaccgagcattcatcttctgtgttcccatcttcttcactctcattctcctctttctcttctatctcttctacctccgaccgcgaactaggctccattggatttcacactttcgccttcccagcaacaacaaccgcaataatgccatctccacattgggtttgggcttgaacaaagaacttagagagatgctgcccattattgtctacaaggaaagcttctccgtcaaagatactcaatgctcagtgtgccttttggactaccaggcagaggataggctgcaacaaatacctgcatgtggccatacatttcatatgagctgcattgatctttggcttgccacccataccacctgtcctctctgccgcttctccctactaaccactgctaaatcttcaacgcaggcatccgatatgcagaacaatgaagaaacacaagccatggaattctctgaatcaacatctcctagggatctagaaaccaatgtcttccaaaatgtctctggagaagttgccatcagcactcactgcattgatgttgaagggcaaaatgtgcaaaacaatcaataggagcatgatgatgcaaaactctttcaggtgtatcaagttgataatcaattctactatcaaaatgatgaaatccagatatattgacaaacttatcccttccaactcagttgaatgaagcctccagagtgtgcgcagcaactgcacagattgatacttcggcaagaaatgtcttcattcggggaactacagctttgatggtacatttgaattgactcatcattattgtaacttatggtaccctgaatgtgtcttttaagcattctaattttggttaatgtacctaagatagtttacatcacaagtgaaaagtattttatg

SEQ ID NO: 16 - protein, 219 aa, Glycine max
MSECGCSESDPSCGCWSSSSSRSVASTELKLYRAFIFCVPIFFTLILLFLFYLFYLRPRTRLHWISHFRLPSNNNRNNAISTLGLGLNKELREMLPIIVYKESFSVKDTQCSVCLLDYQAEDRLQQIPACGHTFHMSCIDLWLATHTTCPLCRFSLLTTAKSSTQASDMQNNEETQAMEFSESTSPRDLETNVFQNVSGEVAISTHCIDVEGQNVQNNQ

SEQ ID NO: 17 - DNA, 860 bp, Glycine max
  CDS (37)..(567): RING H2 zinc finger protein (GM52750153)
ggtaccaatttggtgaccacggtcattgggtttgggatgagtgccactttcattgtgtttgtgtgcaccagaatcatttgtgggaggctaagagggggtgttgaatctcggatgatgtacgagattgaatcaagaattgatatggaacagccagaacatcatgttaatgaccctgaatccgatcctgttcttcttgatgcaatccctactttgaagttcaaccaagaggctttcagttcccttgaacacacacagtgtgtaatatgtttggcagattacagagaaagagaagtattgcgcatcatgcccaaatgtggccacacttttcatctttcttgcattgatatatggctgaggaaacaatccacctgtccagtatgccgtctgccgttgaaaaactcttccgaaacgaaacatgtgagacctgtgacatttaccatgagccaatcccttgacgagtctcacacatcagacagaaacgatgatattgagagatatgttgaacctacacctactgcagccagtaactctttacaaccaacttcaggagaacaagaagcaaggcaatgatcttagagaactaaaggggttgttctgctcaaaaagagaagaatgtagaatttctgcttctatagaggaatgcttctaattatagattggattcaaattctttgtctgtaatatggccttcatattcacttggtggtgtaaatatgtttccttttgtagcatatgcgggccaaggttttggtggaatttcttgcataccgatttgaagttcttttgtctatggtatcgcttactcaagcaagcacactgctcttgttaatgcttaacagattaaacaaatggttgattac

SEQ ID NO: 18 - protein, 177 aa, Glycine max
MSATFIVFVCTRIICGRLRGGVESRMMYEIESRIDMEQPEHHVNDPESDPVLLDAIPTLKFNQEAFSSLEHTQCVICLADYREREVLRIMPKCGHTFHLSCIDIWLRKQSTCPVCRLPLKNSSETKHVRPVTFTMSQSLDESHTSDRNDDIERYVEPTPTAASNSLQPTSGEQEARQ

SEQ ID NO: 19 - DNA, 755 bp, Physcomitrella patens
  CDS (32)..(679): GTP-binding protein (EST548)
atcccgggagtggcaggctgtaactagcgtcatggccgcaggtggatcaagagcccgagccgattacgattaccccatcaagttgctgttgattggcgacagtggggttgggaaatcttgtcttctccttcgtttctcggatgactcctttactacaagtttcatcaccacaatagggattgacttcaagatacggaccatcgagctggatgggaagcgcatcaagcttcagatatgggacacggctggacaagaacgtttccgcacaatcacaacagcttactacagaggtgccatgggaatattgctggtatacgatgtaacggacgaatcttcatttaacaatattcggaactggatcaggaacatcgagcagcatgcatctgacaatgtgaacaagatcttggttggaaacaaagctgatatggacgagagcaaaagagctgtcccaactgccaaaggtcaagccctagctgatgaatatggcatcaagttttttgaaactagcgctaaaacaaacatgaacgtggaagatgttttcttcacaattgcaagggacatcaaacagaggttggctgagactgattcgaagcctgaggctgctaagaatgcaaagccagatgtcaagcttcttgcaggaaattctcagcaaaagccagcttctagttcctgctgctcgtagctgaaagcttatgttgagacatttgtctggtaagcttttggatctattccgagtaaaggctgtctgagctcgc

SEQ ID NO: 20 - protein, 216 aa, Physcomitrella patens
MAAGGSRARADYDYPIKLLLIGDSGVGKSCLLLRFSDDSFTTSFITTIGIDFKIRTIELDGKRIKLQIWDTAGQERFRTITTAYYRGAMGILLVYDVTDESSFNNIRNWIRNIEQHASDNVNKILVGNKADMDESKRAVPTAKGQALADEYGIKFFETSAKTNMNVEDVFFTIARDIKQRLAETDSKPEAAKNAKPDVKLLAGNSQQKPASSSCCS

SEQ ID NO: 21 - DNA, 880 bp, Glycine max
  CDS (154)..(705): GTP-binding protein (GM50181682)
ggaagggaaggaggagagggagagggagagagaaagaaaggtgaattggattgcatctctctctgtgtgttggaagaggggaatcgtagatctgatttctttctttctttttaataattttgtgatcagaattattgagctgaacaaaagacaatgggattgtgggaagcttttctcaattggcttcgcagcctttttttcaagcaggaaatggagttatctctaataggacttcagaatgctgggaagacttcccttgtaaatgtagttgctaccggtggatatagtgaggacatgattccaactgtgggattcaatatgaggaaagtgacaaaagggaatgttacaataaagttatgggatcttggagggcaacctaggttccgcagcatgtgggaacgttactgtcgtgccgtttctgctattgtttatgttgttgatgctgccgatccagataaccttagcatatcaagaagtgagcttcatgatttgctgagcaaaccatcattgggtggcatccctctgttggtattggggaacaagattgacaaagcgggggctctgtctaaacaagcattgactgaccaaatggatttgaagtcaattactgacagggaagtttgctgcttcatgatctcgtgcaaaaactcgaccaacatcgactctgttattgactggcttgtaaagcattccaaatcaaagagctgagagcctactttctgttttgaactctagtgtaatttatgggtgacacattttctggatttactagaggcatttgcatgtctaactcggttgctgattgatttgtttttcccttttgtcagatgctttgtaatataatatcacatcattcttgtccaatagggagttaaacggg

SEQ ID NO: 22 - protein, 184 aa, Glycine max
MGLWEAFLNWLRSLFFKQEMELSLIGLQNAGKTSLVNVVATGGYSEDMIPTVGFNMRKVTKGNVTIKLWDLGGQPRFRSMWERYCRAVSAIVYVVDAADPDNLSISRSELHDLLSKPSLGGIPLLVLGNKIDKAGALSKQALTDQMDLKSITDREVCCFMISCKNSTNIDSVIDWLVKHSKSKS

SEQ ID NO: 23 - DNA, 996 bp, Hordeum vulgare
  CDS (159)..(731): GTP-binding protein (HV62638446)
ccggctccgacttcggccagaggaaggaaggcaggcaagggcggggacgatcgagccttccccgaaccccgcgcgcatcccataaccttccactagccgttccattctcatcctcttcggcggccgaccagccggccagattctcctgatccagggttatgggtcaggccttccgcaagctcttcgatgccttcttcggcaacaaggagatgcgggtggtgatgcttgggttggatgcagccggtaaaaccaccatactctacaagctacacattggcgaagtactctccaccgttcccactattggcttcaatgttgagaaggttcagtacaagaatgtagtattcactgtgtgggacgtgggtggccaggagaaattgaggcccttgtggaggcactacttcaacaacacagatgctctgatctatgtggtcgattccctcgacagggatagaattggaagagccagggctgaatttcaggccataatcaatgacccgtttatgctcaacagtgtattattggtgtttgctaacaagcaagacatgaggggagcaatgactccgatggaagtatgcgagggtcttggtctgtacgacctgaacaatcgtatctggcatatccaaggtacctgtgctcttaaaggcgatggcctgtatgaaggcttggactggctagcgacgaccctggatgaaatgcgagctacagggcggttagcttcgacatcggcgtaaagagtaacagggaaggaccgtctgtgtttcttggcccctcatttttcctttttgtgtctgccctgtggccgctttttgatgtgttcgacagatttgttgtagtatgaatgattcacaagaggagatgcgttttctgaagagggggtcatcctcttagttggaggcgcatatatattctgttctactctaggattgtgggatgtaaatactgatgtttctactgatggcatgacacgcttaatatttgtggtttagtctgaag

SEQ ID NO: 24 - protein, 191 aa, Hordeum vulgare
MGQAFRKLFDAFFGNKEMRVVMLGLDAAGKTTILYKLHIGEVLSTVPTIGFNVEKVQYKNVVFTVWDVGGQEKLRPLWRHYFNNTDALIYVVDSLDRDRIGRARAEFQAIINDPFMLNSVLLVFANKQDMRGAMTPMEVCEGLGLYDLNNRIWHIQGTCALKGDGLYEGLDWLATTLDEMRATGRLASTSA

SEQ ID NO: 25 - DNA, 917 bp, Triticum aestivum
  CDS (107)..(748): GTP-binding protein ()TA56528531)
acggacgaagcggagatcgatcggacgaacgccgccgccgcatcggagcacgcgcgcgcgcgagcgaagccgtccccgcctcgctcggcctgggagttagggcgcgatggcggcgccgccggctagggctcgggccgactacgactacctcatcaagctcctcctcatcggcgacagcggtgttgggaaaagttgtctgcttctgcggttctcagatggctccttcaccactagcttcatcaccactattggtattgacttcaagataaggactgttgagttggatggtaagcggattaagttgcagatctgggatactgctggccaagaacgctttcggactataactactgcctactacaggggagcgatgggcattttacttgtttatgatgtcacggacgaggcgtcattcaataacatcagaaattggatcaaaaacattgaacagcatgcttcagataacgtgagcaaaattttggtggggaacaaagcggatatggatgaaagcaaaagggctgttcccacttcaaagggccaggccctggccgatgaatacgggatccagttctttgaagcgagtgcaaagacaaacatgaatgtcgagcaggttttcttctctatagcaagagacatcaaacagagactctcggaggcagattccaagactgagggggggactatcaagatcaacacggagggtgatgccagtgcagcagcaggacagaagtcggcttgctgtgggtcttgaaccgtcgtcgtcgctacggaaaaaaaaagatagttgcgacacggtgcttgtaattcttgtcattccattctttgcctgctggtttcgttgtgttatttaagttatcgctgttgttaggatttggacaaattggtgttacgtcagcaattacttgcagtatcggtgg

SEQ ID NO: 26 - protein, 214 aa, Triticum aestivum
MAAPPARARADYDYLIKLLLIGDSGVGKSCLLLRFSDGSFTTSFITTIGIDFKIRTVELDGKRIKLQIWDTAGQERFRTITTAYYRGAMGILLVYDVTDEASFNNIRNWIKNIEQHASDNVSKILVGNKADMDESKRAVPTSKGQALADEYGIQFFEASAKTNMNVEQVFFSIARDIKQRLSEADSKTEGGTIKINTEGDASAAAGQKSACCGS

SEQ ID NO: 27 - DNA, 976 bp, Hordeum vulgare
  CDS (183)..(728): GTP-binding protein (HV62624858)
caaatcgccgaagcaactgataggagagaggaagtgggggagagatcttcgtcttcaccactcgcgcgcgcaagctcgctcgctccagatctcccccttccatcgtagatcccacgaccgcaagccgccgcgtccccgacgaaaccctagctcgcgcccctccgccgcgtaggggcgccgccatgggcatcgtgttcacgcggctcttctcgtcagtattcggaaaccgcgaggctcgcatcctcgtcctcggccttgacaatgccggcaagactactatcctctatcggctgcagatgggggaggtcgtctccacgatcccaacaatcggcttcaacgtggagacggtgcagtacaataacatcaagttccaagtttgggatctcggtggtcaaacaagcataaggccgtactggagatgctactttccaaacactcaggctatcatatatgttgttgattcaagtgatactgataggctggtaactgcaaaagaagaatttcattctatccttgaggaggatgagctgaaaggtgcggttgtccttgtatatgcaaataaacaggaccttccaggtgcacttgatgatgctgccataactgaatcattagaacttcacaagattaagagccgccaatgggcaattttcaaaacatctgctataaaaggggagggcctttttgaaggcttgaattggctcagtaacgcactcaagtccggaagcagctaatgcaggctccattccgcgaatcattgcttgatggtaaggaacagggacgatgacatccttctcactagtctgcgcgaaaatcacattctctttatttaactcggaagttatacacaatcagttatctgtagagtgcttgttgaagtttccagatacaacaccaggtgtacccatatcgggagcaagaatatatttgtagaacatactgagcagacttatggtttgaaatctatggcttcaccgcg

SEQ ID NO: 28 - protein, 182 aa, Hordeum vulgare
MGIVFTRLFSSVFGNREARILVLGLDNAGKTTILYRLQMGEVVSTIPTIGFNVETVQYNNIKFQVWDLGGQTSIRPYWRCYFPNTQAIIYVVDSSDTDRLVTAKEEFHSILEEDELKGAVVLVYANKQDLPGALDDAAITESLELHKIKSRQWAIFKTSAIKGEGLFEGLNWLSNALKSGSS

SEQ ID NO: 29 - DNA, 875 bp, Linum usitatissimum
  CDS (100)..(642): GTP-binding protein (LU61640267)
ctcgcgcctcccttctcttcttcgagatccaaagctagggcaaaaaacctttcccacaacacctcctccttcatttcgttctctgtctgtagtttcaagatgggtctatcattcaccaagctgttcagccggctatttgccaaaaaagagatgcggattctgatggtgggtctcgatgcagctggtaagactacaatcttgtacaagctcaagcttggagagatcgtgacaaccattcccaccattggattcaatgttgagaccgtggaatacaagaacatcagcttcactgtctgggatgttggaggtcaagacaagatccgtccattgtggagacactacttccaaaacactcaaggactgatctttgtcgttgatagcaacgatcgcgatcgtgtggtcgaggctagagatgaacttcatcgcatgttgaatgaggatgagttgagggatgcagttctgctagtctttgccaacaaacaggatctcccgaatgccatgaatgcagctgagatcacggacaagcttggccttaattcccttcgtcagcgccactggtacatccagagcacctgcgctacctctggtgaaggactctacgagggactcgactggctgtccaacaacattgccaacaaggcatagaggactgtggtagacttcacgaagccttatgtaactgcttcgatactgccgctagcgcgaacccataatatgatgtttttcgtgtttgttttgaggggtatgtcgatgtatcctgtaatcgtttgcaagtgatgttggtaattctatctttttgtagattctcaaaataataatctttcatacgtattgttaaatatgattctgtaacgtgactcacaagttacctcttt

SEQ ID NO: 30 - protein, 181 aa, Linum usitatissimum
MGLSFTKLFSRLFAKKEMRILMVGLDAAGKTTILYKLKLGEIVTTIPTIGFNVETVEYKNISFTVWDVGGQDKIRPLWRHYFQNTQGLIFVVDSNDRDRVVEARDELHRMLNEDELRDAVLLVFANKQDLPNAMNAAEITDKLGLNSLRQRHWYIQSTCATSGEGLYEGLDWLSNNIANKA

SEQ ID NO: 31 - DNA, 872 bp, Linum usitatissimum
  CDS (39)..(617): GTP-binding protein (LU61872929)
agcagcagggcgcaccggtcggccggccctttcccgatatgttcctattcgactggttctatggaattctcgcatctcttgggctatggcagaaagaggccaagatcctcttcttgggtctcgacaacgccggcaagaccactcttcttcacatgttgaaagacgagagactagtgcaacatcagccgacccagcatcctacttcagaggagttgagtattggcaaaatcaagttcaaagcttttgatttgggcggccatcagatcgctcgccgcgtctggaaagactattatgccaaggttgatgccgtggtctaccttgttgatgcctacgacaaggagaggtttgcagagtcgaagaaggagctggacgccctcttgtcagacgagggccttaccagtgttccattcctgatcctaggcaacaaaatcgacatcccctatgcagcatcggaagacgagctccggtaccatctagggctgtcgaatttcacaaccggaaagggcaaggtgaacctcacggactccaacgtccggcctcttgaggttttcatgtgcagcattgtccggaagatgggttacggagaaggcttcaagtggctctctcagtacatcaagtagaggaattatatcaagatataatagaagatggggttattcagtactttctcctcccctcagctgttctgtatttttgtactggagcttatttcctcatgcccttgcccattactgtttttgtttctgggtttatcgatgttttgttttttgcaagtcagttagatacaattagattggaagaatgggtattcttttgctgctgttatggataaactggattggtgtaaggagattaagcaacttgggagagcc

SEQ ID NO: 32 - protein, 193 aa, Linum usitatissimum
MFLFDWFYGILASLGLWQKEAKILFLGLDNAGKTTLLHMLKDERLVQHQPTQHPTSEELSIGKIKFKAFDLGGHQIARRVWKDYYAKVDAVVYLVDAYDKERFAESKKELDALLSDEGLTSVPFLILGNKIDIPYAASEDELRYHLGLSNFTTGKGKVNLTDSNVRPLEVFMCSIVRKMGYGEGFKWLSQYIK

SEQ ID NO: 33 - DNA, 823 bp, Linum usitatissimum
  CDS (161)..(709): GTP-binding protein (LU61896092)
cccgcctctgctcatacacgattaccacgattactaagttatcttttcattatctctttccctcgcccacccgctgcacctttcgatcattctcccgaatcaacttggattggtaatttttgctttcgatccgtttctcaagggggagtagaagcagaagatgggagcattcatgtctagattttggttcatgatgtttccagctaaggagtacaagattgtggtggttggattggataatgcagggaagaccaccactctttacaaattgcacttgggagaggtcgtcactactcaccctactgtcggtagcaatgtggaagaagttgtctacaagaacattcgtttcgaggtgtgggaccttggaggacaagagaggctgaggacatcatgggcaacatattacagaggaacacatgccataatagtagtgatagacagcacggatagagcaaggatttcgataatgaaggatgaactttttagactgattgggcatgacgaattgcagcagtcggttgtactggtatttgcaaacaaacaagatctaaaggacgccatgactcctgctgagataacagatgcactttcactccacagcatcaaaaatcacgactggcacatccaggcatgttgcgcactcaccggtgaaggcttgtacgacggccttggatggattgcacagcgtgttactggcaaggccccaagttagaagtgaaagttggtgatgaggtggaggaaattatagagagcatcttctttcttgtacaccatctgattgtacttgttcatcaatttactgcaattgtgtttcttgcgactc

SEQ ID NO: 34 - protein, 183 aa, Linum usitatissimum
MGAFMSRFWFMMFPAKEYKIVVVGLDNAGKTTTLYKLHLGEVVTTHPTVGSNVEEVVYKNIRFEVWDLGGQERLRTSWATYYRGTHAIIVVIDSTDRARISIMKDELFRLIGHDELQQSVVLVFANKQDLKDAMTPAEITDALSLHSIKNHDWHIQACCALTGEGLYDGLGWIAQRVTGKAPS

SEQ ID NO: 35 - DNA, 848 bp, Linum usitatissimum
  CDS (63)..(641): GTP-binding protein (LU61748785)
agcaaatcactttcgattctcgcctttaggttttcaattgagttgattgagatagaggagccatgtttctgatcgattggttctacggagttctcgcatcgctcgggctgtggcagaaggaagccaagatcttgttcctcggcctcgataatgccgggaaaaccactctcctccacatgttgaaagatgagaggctagtgcagcatcagccgactcagtacccgacttctgaagagctgagcattgggaaaatcaagttcaaagcttttgatcttggtggtcaccagattgctcgtagagtctggaaagattactatgctaaggtggacgccgtggtctacttggtcgatgcattcgacaaggaaagattcgcagagtccaagaaggaactcgatgcactcctctccgacgagtcactctccaccgtccctttcctgatacttgggaacaagatcgacataccatatgctgcctcggaagacgagttgcgttaccacttggggctcacaaacttcaccaccggcaagggcaaggtgaacttgagtgacacgaatgtccgccccctcgaggtgttcatgtgcagcatcgtccgcaaaatggggtatggcgaagggttcaagtggatgtctcagtacatcaactagaccgtattgtagtgtgttttgtttttgtcttcagacattctcaatggtatttttctacttgttatggtgttcttgttctgagtctggtgttaaaaaatatgtaatatacataaacctgattagagtttggtttttctactgtattgtctgtatcatattttcctactatccaatgcttatagtctttcaagatcttatatctcg

SEQ ID NO: 36 - protein, 193 aa, Linum usitatissimum
MFLIDWFYGVLASLGLWQKEAKILFLGLDNAGKTTLLHMLKDERLVQHQPTQYPTSEELSIGKIKFKAFDLGGHQIARRVWKDYYAKVDAVVYLVDAFDKERFAESKKELDALLSDESLSTVPFLILGNKIDIPYAASEDELRYHLGLTNFTTGKGKVNLSDTNVRPLEVFMCSIVRKMGYGEGFKWMSQYIN

SEQ ID NO: 37 - DNA, 945 bp, Oryza sativa
  CDS (98)..(676): GTP-binding protein (OS34706416)
cctacccaaaacaaaacttcaatttctgtttcagttcgcggagatcaatattttatctaggtccatcgtcgatagaagatacgagaaaccaaaggcaatgtttttgtgggattggttttatgggattctagcgtcgctcgggctgtggcagaaggaggccaagatcttattcttgggcctcgataacgctggcaaaactaccttgcttcacatgctcaaagatgagagattagtccagcatcagcctacccagtatcctacatcggaggagttgagtattgggaagatcaagtttaaagcttttgatctagggggtcatcagattgctcgaagagtttggaaagattactatgcccaggtggatgcagtggtgtacttggttgatgcttatgacaaggagagatttgctgagtcaaaaaaagagctggatgctctactctctgatgaatctttagccagtgtcccttttcttgtccttgggaacaagatagatattccatatgctgcctcagaagaagaattgcgctaccatttgggcctgactaacttcaccacaggcaagggtaaggtaaacttggccgactcaaatgtccgtcccatggaggtattcatgtgcagtattgtgaagaaaatgggttatggggatggtttcaaatgggtttcccagtacatcaaatagtcccttagcaagagatggcttggtacctcatttctagaagtttgtttctctagttgagatttggaggtgttgttgggacaaaattgctgttaaagaaattgcagtatatttcaacttttatttatataaaatgactgggaaccttctcctgttttccccaccctcctacactgtcgatgatgtgctgagcaaatttcagttgatttgtggtgattgatgattttttaggtgaaaaattgaggtggcccgaattattaggcatgctg

SEQ ID NO: 38 - protein, 193 aa, Oryza sativa
MFLWDWFYGILASLGLWQKEAKILFLGLDNAGKTTLLHMLKDERLVQHQPTQYPTSEELSIGKIKFKAFDLGGHQIARRVWKDYYAQVDAVVYLVDAYDKERFAESKKELDALLSDESLASVPFLVLGNKIDIPYAASEEELRYHLGLTNFTTGKGKVNLADSNVRPMEVFMCSIVKKMGYGDGFKWVSQYIK

SEQ ID NO: 39 - DNA, 833 bp, Glycine max
  CDS (93)..(671): GTP-binding protein (GM49750953)
ccaaaacaaaacttcaatttctgtttcagttcgcggagatcaatattttatctaggtccatcgtcgatagaagatacgagaaaccaaaggcaatgtttttgtgggattggttttatgggattctagcgtcgctcgggctgtggcagaaggaggccaagatcttattcttgggcctcgataacgctggcaaaactaccttgcttcacatgctcaaagatgagagattagtccagcatcagcctacccagtatcctacatcggaggagttgagtattgggaagatcaagtttaaagcttttgatctagggggtcatcagattgctcgaagagtttggaaagattactatgcccaggtggatgcagtggtgtacttggttgatgcttatgacaaggagagatttgctgagtcaaaaaaagagctggatgctctactctctgatgaatctttagccagtgtcccttttcttgtccttgggaacaagatagatattccatatgctgcctcagaagaagaattgcgctaccatttgggcctgactaacttcaccacaggcaagggtaaggtaaacttggccgactcaaatgtccgtcccatggaggtattcatgtgcagtattgtgaagaaaatgggttatggggatggtttcaaatgggtttcccagtacatcaaatagtcccttagcaagagatggcttggtaactcatttctagaagtttgtttctctagttgagatttggaggtgttgttgggacaaaattgctgttaaagaaattgcagtatatttcaacttttatttatataaaatgactgggaaccttctcctgttttcctc

SEQ ID NO: 40 - protein, 193 aa, Glycine max; identical to SEQ ID NO: 38

SEQ ID NO: 41 - DNA, 832 bp, Helianthus annuus
  CDS (86)..(628): GTP-binding protein (HA66696606)
ccaaattccacaactcacaacccccctttctctctttctccttcgatccctctccacatccacagggatcctacgcggcaaaaaaatggggctaacgttcacgaaactctttagtcggctgtttgccaagaaggagatgcggatcttgatggtgggtcttgatgcagctggtaagacgaccattttgtacaagctcaagcttggtgagatcgtgacaacgattcctaccattgggtttaacgtggagaccgtggagtacaaaaacatcagcttcaccgtctgggatgtcgggggtcaagacaagatccgtccgttatggaggcactacttccagaacacacaaggtcttatctttgtggttgatagcaatgacagggatagagttgttgaggcaagagatgaattacataggatgttgaatgaggacgagcttcgagatgcagtcttgcttgtgtttgctaacaaacaagatcttccaaatgcaatgaatgctgccgaaatcactgataagcttggccttcattcccttcgccaacgccactggtacatccagagcacctgtgcaacctcaggagagggactttacgagggtctcgattggctttccaataacatcgctaacaaggcataagatgaaacaagaccaaacctaatgtcgatcttggatgctgggagcttttgctttgctctgtgtgtttgttaatgggtagcaaatgtgtctacttatataatatttggctgtattgcagttactttttaaaagcattgtctaaagtttgtaacagaggttaattttgattgttttattatatgatgatgatgtttcttaacc

SEQ ID NO: 42 - protein, 181 aa, Helianthus annuus
MGLTFTKLFSRLFAKKEMRILMVGLDAAGKTTILYKLKLGEIVTTIPTIGFNVETVEYKNISFTVWDVGGQDKIRPLWRHYFQNTQGLIFVVDSNDRDRVVEARDELHRMLNEDELRDAVLLVFANKQDLPNAMNAAEITDKLGLHSLRQRHWYIQSTCATSGEGLYEGLDWLSNNIANKA

SEQ ID NO: 43 - DNA, 846 bp, Helianthus annuus
  CDS (98)..(676): GTP-binding protein (HA66783477)
actccaactgttacagaaataggtcagatccataaacataaccgcttgtgcaactccagatctgtgaacaaattcgatcaattctctcaattcaacgatgtttttgttcgattggttctacggcatccttgcgtcactcggtttatggcagaaggaagcgaagatcttgttccttggcctcgataacgccggtaaaacgacgttgcttcatatgttgaaagacgagagattagttcaacatcaaccgactcaacatccgacgtcggaagaattgagtatagggaagattaagttcaaagcgtttgatttaggaggtcatcagattgctcgtagagtctggaaggattattacgccaaggtggatgccgtagtgtatctagtagatgcatatgataaagaacggtttgccgaatcaaaaaaggaactagatgcacttctttctgacgagaatctgtctgcagtcccctttctgattttaggaaacaagattgatataccatatgcagcctcagaagatgagctgcgttaccaccttggactgacaggggtcacgactggcaaagggaaggtaaatcttcaagattcaagcgtccgccccttggaggtatttatgtgcagcattgtgcgcaaaatgggttacggtgatggtttcaaatgggtctctcaatacatcaaatagtgggcgcctgagcaaatcgagtatcttatctgggaaataaaaaaggtaaggaagaatatggtgatttccccaatttgattttgtattcattctgtaagagtgggattttgtttgtttgtgttggcatgtaaaattctgttagaccaaattgctagttgttttgtttg

SEQ ID NO: 44 - protein, 193 aa, Helianthus annuus
MFLFDWFYGILASLGLWQKEAKILFLGLDNAGKTTLLHMLKDERLVQHQPTQHPTSEELSIGKIKFKAFDLGGHQIARRVWKDYYAKVDAVVYLVDAYDKERFAESKKELDALLSDENLSAVPFLILGNKIDIPYAASEDELRYHLGLTGVTTGKGKVNLQDSSVRPLEVFMCSIVRKMGYGDGFKWVSQYIK

SEQ ID NO: 45 - DNA, 882 bp, Helianthus annuus
  CDS (67)..(675): GTP-binding protein (HA66705690)
ccaaacgaataaccttcacccttggatcactcgcccttgttatataccccctgcaatttctataccatgaatcacgaatatgattacttgttcaagcttttgctgattggggattcgggagtcggcaaatcttgtctcctacttagatttgctgatgactcatatattgacagctacatcagcacaattggtgtggactttaaaatccgcaccgttgagcaggatggaaaaaccattaagcttcaaatttgggacacagctggacaagaaaggttcaggacaattaccagtagctactaccgtggggcccatggcattatcatagtttacgatgttactgacctagacagtttcaacaacgttaagcaatggttgagtgaaattgaccgttatgcaagtgaaaatgtgaataaacttcttgttggaaacaaatgtgaccttacagaaagtagagccgtgtcctatgatactgctaaggaatttgcggataacattggcattccgtttatggaaactagtgccaaagatgctaccaatgttgagcaggctttcatggccatgtcctctgacatcaaaaacaggatggcaagtcagcctggggcaaacaacacgaggccaccttctgtgcagctcaagggtcaacctgttggtcaaaagggcggttgctgctcatcttagaataccagtcttgcagctgtttgattataaagaatcaccatgaatccaactgtcattcaagttttttgctattttattttcatataattcccctataaaagctattatagtttttattatttcaagaatttaatttttttttttaaaattggttgtacaaatttgcaaaaactgtctgctgctagtgttgatttgctattcttt

SEQ ID NO: 46 - protein, 203 aa, Helianthus annuus
MNHEYDYLFKLLLIGDSGVGKSCLLLRFADDSYIDSYISTIGVDFKIRTVEQDGKTIKLQIWDTAGQERFRTITSSYYRGAHGIIIVYDVTDLDSFNNVKQWLSEIDRYASENVNKLLVGNKCDLTESRAVSYDTAKEFADNIGIPFMETSAKDATNVEQAFMAMSSDIKNRMASQPGANNTRPPSVQLKGQPVGQKGGCCSS

SEQ ID NO: 47 - DNA, 942 bp, Triticum aestivum
  CDS (151)..(696): GTP-binding protein (TA59921546)
ccgaagttactctcttcgtcttgagcactcgcgcgcgcaagctcactcgctccagatctccccttaccatcgtgtagatctcacgcccccaagccgccacgcccccaacgagacctagctcgcgcccctccgccgcgtaggggcgccgccatgggcatcgtgttcacgcggctcttctcgtcggtattcggaaaccgcgaggcccgcatcctcgtcctcggcctcgacaatgccggcaagactactatcctctatcggctgcagatgggggaggtcgtttccacgatcccaacgatcgggttcaacgtggagacggtgcagtacaataacatcaagttccaagtttgggatctcggtggtcaaacaagcatcaggccatactggagatgctactttccaaacactcaggctatcatatatgttgttgattcaagtgatactgataggctggtaactgcaaaagaagaatttcattccatccttgaggaggatgagctgaaaggtgcggttgttcttgtatatgcgaataaacaggaccttccaggtgcacttgatgatgctgccataactgaatcattagaacttcacaagattaagagccgccaatgggcaattttcaaaacatctgctataaaaggggaggggttttttgaaggcttgaactggctcagtaatgcactcaagtccggaggcagctaatgtaggaggcccagcctccattccgtgaatcattgcttgatggtaaggaacagggacgatgacagccttctcgctagtctgcgtggaaatcagaatccctttattttaactctggaagttatacacaatcagttatctgtagagtgcttgttgaagtttccagacacaacactaggtgtaccatgtcgagagcaagaatatatttgtagaaaataccgagcaaacgattacggtttgaaatag

SEQ ID NO: 48 - protein, 182 aa, Triticum aestivum
MGIVFTRLFSSVFGNREARILVLGLDNAGKTTILYRLQMGEVVSTIPTIGFNVETVQYNNIKFQVWDLGGQTSIRPYWRCYFPNTQAIIYVVDSSDTDRLVTAKEEFHSILEEDELKGAVVLVYANKQDLPGALDDAAITESLELHKIKSRQWAIFKTSAIKGEGFFEGLNWLSNALKSGGS

SEQ ID NO: 49 - DNA, 915 bp, Hordeum vulgare
  CDS (85)..(726): GTP-binding protein (HV62657638)
cccgccccctcgtctgccggtcggggatcagcaacagcgccgatcgaggggtaggacgaggaggaggaggcgggtgcgcgcgacatggctgcgccgccggcgagggcccgggccgactacgactacctcatcaagctcctcctcatcggggacagcggtgttggcaagagttgcctccttctgcggttctctgatggctccttcactacgagctttattaccacgattggtattgactttaagatcagaacaatagagctggatcagaaacgtattaagctacaaatatgggacacggctggtcaagaacggttccggactattaccactgcgtattaccgtggagccatgggtatcctgcttgtttatgacgtcaccgacgagtcatctttcaacaacataaggaactggatccggaacattgagcagcatgcctctgacaacgtcaacaaaattttgattggcaacaaggctgatatggatgagagtaaaagggctgtacctactgcgaaggggcaagctttggccgatgaatatggcatcaagttctttgaaactagtgccaagacaaacctgaacgtggagcaggttttcttctccattgcccgcgacattaagcagaggcttgccgagaccgattccaagcctgaggacaaaacaatcaagattaacaaggcagaaggcggtgatgcgccggcagcttcgggatctgcctgctgtggctcttaagggatggatgattgagtgtgtcggtgatcattgtttatttgacatcattcggttcccgctgctgctgctgcttgtctgttataggaagaatgtcaatcaagaagaaaactatgacttatgatacagatctggttgtacttatattcgcttcccattctttgaagcaactacccttgcctttgacgg

SEQ ID NO: 50 - protein, 214 aa, Hordeum vulgare
MAAPPARARADYDYLIKLLLIGDSGVGKSCLLLRFSDGSFTTSFITTIGIDFKIRTIELDQKRIKLQIWDTAGQERFRTITTAYYRGAMGILLVYDVTDESSFNNIRNWIRNIEQHASDNVNKILIGNKADMDESKRAVPTAKGQALADEYGIKFFETSAKTNLNVEQVFFSIARDIKQRLAETDSKPEDKTIKINKAEGGDAPAASGSACCGS

SEQ ID NO: 51 - DNA, 869 bp, Brassica napus
  CDS (109)..(726): GTP-binding protein (BN43540204)
gacacgcctaaccgtaacctccttttatttttttcttagaaaacttcttttttcctgggaaaaattcacgaatcaatcggaaaaaactcacgaagagctcgagaaaccatgagcaacgagtacgattatctgttcaagcttctgttgatcggcgactcatccgtaggaaaatcatgcctgcttcttcgattcgctgatgatgcgtacatcgacagttacataagtaccattggtgttgacttcaaaattaggacgattgagcaggatgggaagacgattaagcttcaaatctgggatactgctgggcaggagcgtttcaggaccatcactagcagctactacagaggagctcatggaatcattattgtgtatgactgtaccgagatggagagtttcaacaatgtgaagcagtggttgagtgagattgacagatatgctaatgacagtgtttgcaagcttcttattggtaacaagaatgatatggttgaaagtaaagttgtttccaccgaaactggaaaggccttagccgatgagctcggaataccctttctcgagacaagtgctaaggattctatcaacgtcgaacaggcattcttaactattgctggcgagatcaagaagaaaatgggaagccagacgaatgcaaacaagacatctggaagtggaactgtccaaatgaaaggtcagccaatccaacagaacaatggtggcggttgctgcggtcagtagttaagcaaagtgttatcaaaactatgtgagacttttttttttcttactatgtgctgtgaaaactaatggctgtctaaaacagtaacgctggaaactttgataccatgtcactctatgttcaatctatggtggtagttgcg

SEQ ID NO: 52 - protein, 206 aa, Brassica napus
MSNEYDYLFKLLLIGDSSVGKSCLLLRFADDAYIDSYISTIGVDFKIRTIEQDGKTIKLQIWDTAGQERFRTITSSYYRGAHGIIIVYDCTEMESFNNVKQWLSEIDRYANDSVCKLLIGNKNDMVESKVVSTETGKALADELGIPFLETSAKDSINVEQAFLTIAGEIKKKMGSQTNANKTSGSGTVQMKGQPIQQNNGGGCCGQ

SEQ ID NO: 53 - DNA, 908 bp, Brassica napus
  CDS (58)..(669): GTP-binding protein (BN45139744)
tccaccctccccccccagattttcctctgttcgctgtcatctaaagtcgaaaccaccatgaatcccgccgagtacgactaccttttcaagctcctgctcattggggattctggcgtgggcaagtcttgtctactgttgagattctctgatgattcgtatgtagaaagttacataagcactattggagtcgattttaaaattcggactgtggagcaagacgggaagacgattaagctccaaatttgggacactgctggtcaagagcgcttcaggactattactagcagttattaccgtggcgcacatggaatcattattgtctacgacgtcacagatcaagaaagctttaataatgtgaagcaatggttgagtgaaattgatcgttatgctagtgacaatgtgaacaaactcctagttggaaacaagtgtgatcttgctgaaaacagagccgttccatatgaaaccgcaaaggcttttgccgatgaaattggaattcctttcatggagactagtgcaaaagatgctacaaacgtggaacaggctttcatggccatgtcggcatccatcaaagagagtatggcaagccaaccagctgggaacattgccagaccgccgacggtgcagatcagaggacagcctgttgcccaaaagaatggctgttgctcaacttgattgcctagcaatatccttttccgttcagtcttcgagtcctacaaccttaagccaaaattgttttctcttcagttcacttgtactttgtacgtcatttctggtctgtaattaaggtcacttgtcctttggttggctgtttttctctttgcgtatcaacattttcgtaccaccacatttttgtggctgccttcagtgtatttatatactgtcgttttgcttaacaatgtttattagat

SEQ ID NO: 54 - protein, 204 aa, Brassica napus
MNPAEYDYLFKLLLIGDSGVGKSCLLLRFSDDSYVESYISTIGVDFKIRTVEQDGKTIKLQIWDTAGQERFRTITSSYYRGAHGIIIVYDVTDQESFNNVKQWLSEIDRYASDNVNKLLVGNKCDLAENRAVPYETAKAFADEIGIPFMETSAKDATNVEQAFMAMSASIKESMASQPAGNIARPPTVQIRGQPVAQKNGCCST

SEQ ID NO: 55 - DNA, 1295 bp, Brassica napus
  CDS (583)..(1056): GTP-binding protein (BN3613585)
tccgtcatttccattgatctctctcgttcttctctgctcatcactatcaccacggtcctcttctctgcctcgtttgatccgattcgatttcgatggcagctccacctgctaggggtagagccgattacgattacctcataaagcttctcctgatcggtgatagcggtgtgggcaaaagttgtttgctgttaaggttctctgatggctcattcaccactagcttcatcaccaccattgggtttgtattatctttaagaatctattagagactatggtgatgcatgatgtttcacactgactctctttggtgtttgtgtgttggcttataatgatgcagcattgattttaagattagaactattgagcttgatactaaacgcatcaagctccagatttgggatactgctggtcaagaacgttttcgaaccatcaccactggttagtcagtggaaattggattagagaggattaagagtcactagcagtctacttaatgctatggatgatgctttgaggatatttagtttttttttttttttttgaaaactgataagtaccattgcagcttattaccgaggggcaatgggcattttgctggtctatgatgtcacagacgagtcatcctttaacagtaacttttgcttctgtctaagcattgacatcttttattttatttacatttttgctctgttctggacctgttttcttgaccttgttgcagatattaggaactggattcgtaatattgaacagcacgcttcggataatgttaataaaatcttggtagggaacaaagccgatatggatgagagcaagagggctgttccaacatcaaagggtcaagcacttgctgatgaatatggaatcaagttctttgaaacaagtgccaaaacaaatctaaatgtggaagaggttttcttctcgatagcaaaggacattaagcagagactcacagatactgactcgagagcagagcctgcgacgattaggataagccaaacagaccaggctgctggagccggacaagccacgcagaagtctgcatgctgtggaacttaaaagttactcaagttgaagtgaagtgcaaagaaaccagatttgtgccaaatcatttgtcttgtctttggtgcttttgtatttttttttctcttttgatgattgttctaaatttgccatttttagtttagattcgatggccctatagctgattcagtggcttttgattgttaacacttttgctcacaactcaaaatctcttgcactctctgttaataaagcttttccctttgcagcac

SEQ ID NO: 56 - protein, 158 aa, Brassica napus
MGILLVYDVTDESSFNSNFCFCLSIDIFYFIYIFALFWTCFLDLVADIRNWIRNIEQHASDNVNKILVGNKADMDESKRAVPTSKGQALADEYGIKFFETSAKTNLNVEEVFFSIAKDIKQRLTDTDSRAEPATIRISQTDQAAGAGQATQKSACCGT

SEQ ID NO: 57 - DNA, 962 bp, Linum usitatissimum
  CDS (104)..(709): GTP-binding protein (LU61965240)
ttttccacccaatttctctcccaactccgattcgccggcgtagcttcgtccgcctccgacgagttcgagcccgatctccttaaccgccgacaacgtcatcatcatgaacactgaatacgattacttgttcaagcttttgcttattggagattctggagtcggcaaatcgtgtctgcttttgagattcgctgatgattcgtaccttgacagctacatcagtaccataggagtcgatttcaaaatccgcactgtggagcaggatgggaagaccatcaaactccaaatttgggacacagcagggcaagagcgatttaggacgatcaccagcagttactacaggggtgctcacgggatcattgttgtttatgatgtcacggaccaagagagtttcaacaacgtaaaacagtggctgaacgagatcgatcgctacgctagcgagcacgtgaacaagcttcttgtgggaaacaagagtgacctcactagcaacaaagtcgtttcgtatgaaacagggaaggcattagctgatgaactcggtatcccgttcatggagacgagtgccaagaacgcgtccaacgtagaagacgctttcatggccatgtcagctgcaatcaagaccaggatggctagccagcccacgaacaatgccaagccaccgactgtccaaatccgtggagaaccggtcaaccagaagtcaggctgctgttcttcttgaacagcatggattgggatcgtacggtgatgttaatcgtgttcggctaatccttgtggcatgtaaacttggtttcaatattcttattggttttccatatgaacgacaggattattcgtttcgttttcgccttcctgtttttttagtcgcacgtcacatttacagattctgtcgaaacttcgctctttaatgtaattcgattccaggtctgaacaaaacatttgtacaaagtagggaattctgttgaaatgtg

SEQ ID NO: 58 - protein, 202 aa, Linum usitatissimum
MNTEYDYLFKLLLIGDSGVGKSCLLLRFADDSYLDSYISTIGVDFKIRTVEQDGKTIKLQIWDTAGQERFRTITSSYYRGAHGIIVVYDVTDQESFNNVKQWLNEIDRYASEHVNKLLVGNKSDLTSNKVVSYETGKALADELGIPFMETSAKNASNVEDAFMAMSAAIKTRMASQPTNNAKPPTVQIRGEPVNQKSGCCSS

SEQ ID NO: 59 - DNA, 1055 bp, Linum usitatissimum
  CDS (4)..(6): GTP-binding protein (LU62294414)
ccgaaattgaccccgttctgtttgtgagatctttttgatcattattagccagacagaaacggtgcattaacagttgttgagaggaaaagcaaagcaaaagcaggaacaagaggaagaagcaagagagaaagaaagcttgcttcttttttttctgttttctgttccatttgggtggctgctgctggaatttgggaggagaaatttagttctggaatgggatcttcttcaggtagtagtgggtatgatctgtcgttcaagttgttgttgattggagattcaagtgttggcaaaagcagcctgcttgtcagcttcatctccaccacctctgctgaagaagatcttgctcccaccattggtgtggacttcaagatcaagcagctgacagtagctggcaagagattgaagctcaccatttgggatactgctgggcaggagaggttcaggacactaacaagctcttactacaggaatgcacagggtatcatacttgtttatgacgtgaccaggagagagacctttacgaacctatcggacgtatgggctaaagaagttgagctctactgcacaaaccaggactgtgtcaagatgcttgttggcaacaaagttgacaaagactctgacagaactgtaaccagagaagaaggaatggaacttgcaaaagagcgtggatgtttgttcctcgagtgcagtgccaaaactcgtgaaaacgtggagcaatgcttcgaggagcttgcgcaaaagataaaggatgttccaagtctcttggaagaaggatctacggccgggaagaggaacattctaaagcaaaacccagatcgccaaatgtctcaaagcaacggctgttgctcttaaataatgattgactaactgattgatgtatattcagcttcagttctttacctttgtttcttctgtttgtgatttcgagggtgtgtatttcccagagtttccgattagtttgttgcaaaagattggtttgatgaggctaacggtgaatccagtcgagtcgtcaatgaacgaatgtgatatgatatatataggtttgtaattgatgt

SEQ ID NO: 60 - protein, 212 aa, Linum usitatissimum
MGSSSGSSGYDLSFKLLLIGDSSVGKSSLLVSFISTTSAEEDLAPTIGVDFKIKQLTVAGKRLKLTIWDTAGQERFRTLTSSYYRNAQGIILVYDVTRRETFTNLSDVWAKEVELYCTNQDCVKMLVGNKVDKDSDRTVTREEGMELAKERGCLFLECSAKTRENVEQCFEELAQKIKDVPSLLEEGSTAGKRNILKQNPDRQMSQSNGCCS

SEQ ID NO: 61 - DNA, 1019 bp, Linum usitatissimum
  CDS (153)..(800): GTP-binding protein (LU61723544)
ggtacctgaagaagaaggcctttccctcttcattctgcattttcttttcctctttggcttttccattagatcttcctcttctgcttcttcctgatctggttttcctctggaattttctgatttagagagtaaatttgttagcgtttgaatcaatggctgctccgcccgcaagagctcgtgccgattatgattaccttataaagctcctcctgatcggcgatagcggtgtgggtaagagttgcctcctcctacgtttctcagatggttccttcaccactagtttcattacgaccattggtattgatttcaagataaggacaattgagcttgatggaaaacggatcaagttgcaaatatgggatactgctggtcaagagcgtttccgcactattacaactgcttactatcgtggagcaatgggtattttgctcgtgtatgatgtcactgatgagtcatcattcaacaatatcaggaattggattcgcaacattgaacaacatgcctctgataatgtgaacaagatcttggttggaaacaaagccgatatggatgagagcaaaagggcggttcctaccgcaaagggccaggctcttgcagacgaatacggcatcaagttctttgagacgagtgcaaagacaaacttaaacgtggaggaggttttcttctcaatagccagagacatcaagcaacgacttgcagatacggattcaaagtccgagccacagacgatcaagattaaccagccggaccaggcgggtggttcgaaccaggctgcacaaaagtctgcttgctgtggttcttagagattaagacagaaggaataagagtaatatccaattcccttttggccttgtgcgaaattcaaactcgatactattcgtcttctccctcttcaatctcgtctccacgttttcttcgtcattcttgtttcgcttaattttcgtatgaggttagcgcgacaaagagggctgcgattgtttcaccccttctgaaccttaatgtttttgttgcttccttcc

SEQ ID NO: 62 - protein, 216 aa, Linum usitatissimum
MAAPPARARADYDYLIKLLLIGDSGVGKSCLLLRFSDGSFTTSFITTIGIDFKIRTIELDGKRIKLQIWDTAGQERFRTITTAYYRGAMGILLVYDVTDESSFNNIRNWIRNIEQHASDNVNKILVGNKADMDESKRAVPTAKGQALADEYGIKFFETSAKTNLNVEEVFFSIARDIKQRLADTDSKSEPQTIKINQPDQAGGSNQAAQKSACCGS

SEQ ID NO: 63 - DNA, 925 bp, Linum usitatissimum
  CDS (73)..(675): GTP-binding protein (LU61871078)
aggaactcaattcccttccatctccagacggaattcattcattgagagcaagaaaccctatcatcttcaatcatgggcaccgaatacgactatctcttcaagcttctgctaatcggcgactcctccgttggaaaatcttgcctgctgctccgatttgctgatgattcgtacgttgacagctacatcagtactataggagttgatttcaaaatcagaactgtggagctggatggaaagacggtcaagcttcagatctgggatactgctggtcaggagcgctttagaacaataacaagcagttattaccgaggggcacatggaatcatcattgtctatgatgttactgacatggacagcttcaacaatgtcaaacaatggttaaatgagattgaccgatatgcaaatgatactgtatgcaagcttttggttgggaacaaatgcgatcttgttgagaacaaagttgtcgatacgcagacagcaaaggcgttggccgatgagctaggcatcccttttctggagaccagtgccaaagattcaataaatgtggaacaagctttcttaacaatggctgcagaaattaagaaaaaaatgggtaatcaaccgacagctagcaaggcgaccggaacggttcagatgaaaggacaaccgatccagcaaagcaacaactgctgtggttaaacctagtcgggctattttgatgtcctgggataagactagtgtggtgaaagtttgtttccatggtttctaggttttctaacttgatgaagtttagagcaaggtgtagtagattcagttccagataatgtatctccttataatgcttgtaatctatgtgaactgcgatccaatcgagtcgttatccgagtagatctcaactgttgtccgttccccagaattcaactggtttaaaatgttgcctttctgc

SEQ ID NO: 64 - protein, 201 aa, Linum usitatissimum
MGTEYDYLFKLLLIGDSSVGKSCLLLRFADDSYVDSYISTIGVDFKIRTVELDGKTVKLQIWDTAGQERFRTITSSYYRGAHGIIIVYDVTDMDSFNNVKQWLNEIDRYANDTVCKLLVGNKCDLVENKVVDTQTAKALADELGIPFLETSAKDSINVEQAFLTMAAEIKKKMGNQPTASKATGTVQMKGQPIQQSNNCCG

SEQ ID NO: 65 - DNA, 1019 bp, Linum usitatissimum
  CDS (165)..(773): GTP-binding protein (LU61569070)
tgaaactctctctctctctctctctctctctctctctctctctctcgtcttcaacaacaacagaaaacatcgccgctgttcgcttcacatctactccggcgtagctcgatctacgacggttttaggtttcgcttccttctccacgcgttcgtcagctcgccatcatgaactctgagtacgattacttgttcaagcttttgcttatcggagattccggagtcggcaagtcatgtctacttttgcgattcgctgatgattcgtacttggacagttacatcagtaccatcggagtggacttcaaaattcgcaccgtggagcaggatggcaaaaccattaagctccaaatctgggatacggcagggcaagaacgattcaggaccattacaagtagttactatcgtggtgctcatgggattattgtggtctatgatgtcacagaccaagagagtttcaacaatgtcaaacagtggttgagtgaaattgatcgctacgcaagtgagaacgtgaacaaacttctagttgggaacaagagtgacctcactgccaacaaagttgtttcatatgaaactgctaaggcatttgccgatgaaattgggattcccttcatggagacgagtgccaagaacgcttccaatgtcgaagatgcttttatggcaatgtcagctgcaatcaagaccaggatggctagccaacctgtgtcaggcactgccagacctccaacggtgcaaatccgcggagaaccagtgaaccagaagtcaggttgctgctcttcttgaaaagtagaagcggtggtagtggtgttgggtctctgaagcttaattgtgtgtcctttattatgaatgacatgtaaaactagttctcactgttgttactgcttttgatgtgaaaaaggatttatttgcatcttttctatttcttgggtcagtttcagtaatgtgttgaaactttgattgttttaaatgtaatttggtttcaggacaacatttgtacaaattagaaatactgttttgttgaacgcc

SEQ ID NO: 66 - protein, 203 aa, Linum usitatissimum
MNSEYDYLFKLLLIGDSGVGKSCLLLRFADDSYLDSYISTIGVDFKIRTVEQDGKTIKLQIWDTAGQERFRTITSSYYRGAHGIIVVYDVTDQESFNNVKQWLSEIDRYASENVNKLLVGNKSDLTANKVVSYETAKAFADEIGIPFMETSAKNASNVEDAFMAMSAAIKTRMASQPVSGTARPPTVQIRGEPVNQKSGCCSS

SEQ ID NO: 67 - DNA, 905 bp, Oryza sativa
  CDS (97)..(705): GTP-binding protein (OS34999273)
ttttcccttccgttggtgccattcgtgcagcaccggatcctctcatttctccggcgataactctcccttttccggcgaattcaccgcttcctcgatatgaatcccgagtatcactatctgttcaagctccttctgattggagactctggtgttggtaaatcatgccttcttctaagatttgctgatgattcatacattgagagctacataagcaccatcggagttgattttaaaattcgcactgttgagcaggatgggaagacaattaaactacagatttgggatactgctggacaagaacgatttaggacaataactagtagctactatcgtggagcacatggaatcattattgtttatgacgtgacagatgaagatagcttcaataatgtgaagcaatggctcagtgaaattgaccgctatgccagtgataatgttaacaaacttttggttggaaacaagagtgatctgacagcaaatagagttgtctcatatgacacagctaaggaattcgcagatcaaattggcatacctttcatggaaacaagtgcaaaagatgctacaaatgtggaagatgctttcatggccatgtctgctgccatcaagaatagaatggctagtcagccttcagcaaacaatgcaaggcctccaacagtgcagatcagagggcaacctgttggacaaaaaagtggttgctgctcttcctaaccaggtggtgctgcttggtctacacttaccttttgcatgtaaggggcatatgctatttcactaaatagtggaccagtgtcacgtaatccaacctgtggtttgggaattggcctagatgatcccattctttaccatatacttgaatgctatgattgtgcttagtacttgttaatgataaaacttttatatttctgctc

SEQ ID NO: 68 - protein, 203 aa, Oryza sativa
MNPEYHYLFKLLLIGDSGVGKSCLLLRFADDSYIESYISTIGVDFKIRTVEQDGKTIKLQIWDTAGQERFRTITSSYYRGAHGIIIVYDVTDEDSFNNVKQWLSEIDRYASDNVNKLLVGNKSDLTANRVVSYDTAKEFADQIGIPFMETSAKDATNVEDAFMAMSAAIKNRMASQPSANNARPPTVQIRGQPVGQKSGCCSS

SEQ ID NO: 69 - DNA, 905 bp, Helianthus annuus
  CDS (67)..(675): GTP-binding protein (HA66779896)
gccacctgcaacaaaatctccacaaatctttcactcaaccgatcacaactccacacacaaacaaagatgaatcccgaatacgactatctgttcaagcttttactcattggagattcaggagttggaaaatcatgtctcctattgcgttttgctgatgattcgtacttggaaagttacattagcaccattggggttgactttaaaattcgcactgtggaacaagatggcaaaacaattaagcttcaaatttgggatacagctggacaagaacgtttcaggaccatcactagcagctactatcgtggagctcatggcattattgttgtttatgacgtgacagatcaagagagtttcaacaacgtgaaacaatggttgagtgaaatcgatcgttacgctagtgagaacgtaaacaagcttcttgtcggaaacaaatgcgatcttacgtctcagaaagctgtttcctacgaaacaggaaaggcgtttgctgatgagatcgggatcccgtttctcgaaacaagtgccaagaattccaccaatgtcgaagaggcgtttatggctatgactgctgaaataaaaaacaggatggcaagccagccggcaatgaacaatgctagaccgctaactgttgaaatccgaggtcaaccggtcaaccaaaagtcaggatgctgctcttcttgaagagggtaaggatgtgggtggtcaacgtgtgttaagatatgcatttttgttcactcatacttgtcgatgtgaagaagccatttcgttgatcgccaaacttttgtcattcttttcgatgaattcggggaccttttgtacaaagtaggataagactgttgaatgtgtattatgttatactgttttgctgtttgcatttcctttacattttaatgacatttcaagtgtgt

SEQ ID NO: 70 - protein, 203 aa, Helianthus annuus
MNPEYDYLFKLLLIGDSGVGKSCLLLRFADDSYLESYISTIGVDFKIRTVEQDGKTIKLQIWDTAGQERFRTITSSYYRGAHGIIVVYDVTDQESFNNVKQWLSEIDRYASENVNKLLVGNKCDLTSQKAVSYETGKAFADEIGIPFLETSAKNSTNVEEAFMAMTAEIKNRMASQPAMNNARPLTVEIRGQPVNQKSGCCSS

SEQ ID NO: 71 - DNA, 1001 bp, Oryza sativa
  CDS (136)..(762): GTP-binding protein (OS32667913)
ctcaccaccttcttgttcctggagaacctcctctccagctctgtccaagcatcaattctctttcttttgcttcctgctgatacctttgatcctgagcagaagaagctgcagaagtgggttaaggcaggaagagccatgaacaacgaatttgattacctgttcaagctgctcctcatcggcgactcctcggtcggcaagtcatgcttcctcctccgattcgcggacgactcctacgtcgacagctacatcagcacgatcggtgttgacttcaagattcgcacgatcgagatggacgggaagaccatcaagctgcagatctgggacacagcaggacaggagcgattcagaaccatcaccagtagctactaccggggagctcatgggataattatcgtctatgacattacggatatggagagcttcaacaatgtgaaggagtggatgagcgagatcgacaagtacgccaatgacagcgtatgcaagcttcttgttggtaacaagtgtgatctggcagagagcagagttgttgaaactgcagtagcacaggcttatgctgatgagataggcattccattccttgaaacaagtgctaaggactcgatcaatgtcgaagaggctttcttggctatgtgtgccgcaatcaaaaagcaaaaatctgggagccaggcagccctggagaggaaggcatccaatctagttcagatgaaaggtcagccaattcagcaacagcagcagccacagaagagcagctgttgttcatcgtgatggcacaatggtctggcatcttccatgaattgggatgaacatggcatatctgttaagtgtgttcctctgtcttctcatagatttgagcactttagttactgcaaggtgtcgccacatctgttgaaaatcgagtcaagaacctaatttcctgtctttgatgattctctaataaacattgcatctagaaagttgtaccatatttaatagatacatgtagtttccagtctgaaaggtcg

SEQ ID NO: 72 - protein, 209 aa, Oryza sativa
MNNEFDYLFKLLLIGDSSVGKSCFLLRFADDSYVDSYISTIGVDFKIRTIEMDGKTIKLQIWDTAGQERFRTITSSYYRGAHGIIIVYDITDMESFNNVKEWMSEIDKYANDSVCKLLVGNKCDLAESRVVETAVAQAYADEIGIPFLETSAKDSINVEEAFLAMCAAIKKQKSGSQAALERKASNLVQMKGQPIQQQQQPQKSSCCSS

SEQ ID NO: 73 - DNA, 835 bp, Helianthus annuus
  misc_feature (1)..(835): GTP-binding protein gene (HA66453181)
tgtcccccaattctctctctctctctctctctcatcggagcttcaccaccgccggtgatccacaacattcgctatatacctttctccgatcactatcaacagccatgactcctgagtatgactacctgttcaagcttttgctcattggagattcgggtgtaggaaagtcatgtctacttctgaggtttgctgacgattcttacttggacagttacataagcaccatcggagtcgattttaaaattcgtaccgtggagcaagatgccaaggttatcaagcttcaaatttgggatactgctggccaagaacgttttaggacaatcacaagcagctactatcgaggagcacatggcatcatcgtggtttatgatgtgacggaccaagagagctttaataacgttaagcagtggctgagtgaaatcgaccgttacgctagtgagaacgttaacaagatccttgttggaaacaaatgcgatcttgttgcaaataaagtcgtttcaaccgaaacagccaaggcatttgctgatgaaattggaattccgttcttggaaacaagtgcaaaagatgcaaccaatgtcgaacagggtcaaccggtctcccagaacagcggatgctgctcttagtggttgtatttgatgggggtgatgtggcggtgtacaagtattgtccttgtgttactttcatggccatgacggcttccatcaaagacaggatggcgagtcaacccaatttgaatacctcaaagcctccaacggtcaacattcgtggggttggattctttttactttctttgtttcagattgtttgcattgtataaaattcaagaattcttttt

SEQ ID NO: 74 - protein, 202 aa, Helianthus annuus
  MISC_FEATURE (1)..(202): GTP-binding protein (HA66453181)
MTPEYDYLFKLLLIGDSGVGKSCLLLRFADDSYLDSYISTIGVDFKIRTVEQDAKVIKLQIWDTAGQERFRTITSSYYRGAHGIIVVYDVTDQESFNNVKQWLSEIDRYASENVNKILVGNKCDLVANKVVSTETAKAFADEIGIPFLETSAKDATNVEQAFMAMTASIKDRMASQPNLNTSKPPTVNIRGQPVSQNSGCCS

SEQ ID NO: 75 - DNA, 938 bp, Helianthus annuus
  CDS (37)..(627): GTP-binding protein (HA66709897)
agaaaccaatcatccaccgacaccgtcacaatgagcaacgaatacgattatctcttcaaacttttactcatcggtgactcctccgtcggaaaatcatgccttcttctccgatttgctgatgattcttatgtggatagttacataagcacaattggagttgactttaaaattaggactgtggagcaggataggaagaccatcaagctgcagatatgggatactgctggccaggagcggtttcggactataacaagcagttactacagaggagcacatggaataattatcgtgtatgatgtgactgagatggagagcttcaacaatgtgaagcaatggctgagtgaaatcgacagatatgcaaatgaatcagtctgcaagcttcttgttggaaacaaatgtgatctagttgagaacaaggttgttgacacacaaacagctaaggcatttgcagatgagctcgggatccctttcctcgagaccagtgcaaaagactccgtaaacgtggaacaggctttcttgacaatggctgcagagataaagaaaaaaatgggtaaccagccaacgggcgacaagagcatagttcaaatcaaagggcagccgattgagcagaagagcaattgttgtggttaatactgttaaggtccgcaggacaactggtaaaaatgtttgtaaaatgttgttggcttttaattagcttcatggacttttttgtatcatctgatttcaactacgggtaattttctgcatcaaattactttgaaaggtggcaaaatgagcatggttgtgtgacgggtcacaacaggttaaaaaggtcgggccgccgacttgaaacgcttttgatctagttttcgttctattacactttgaaatactatcccaataattttttttggattaattagattataagcttacattgctcgacgttggtttatatc

SEQ ID NO: 76 - protein, 197 aa, Helianthus annuus
NEYDYLFKLLLIGDSSVGKSCLLLRFADDSYVDSYISTIGVDFKIRTVEQDRKTIKLQIWDTAGQERFRTITSSYYRGAHGIIIVYDVTEMESFNNVKQWLSEIDRYANESVCKLLVGNKCDLVENKVVDTQTAKAFADELGIPFLETSAKDSVNVEQAFLTMAAEIKKKMGNQPTGDKSIVQIKGQPIEQKSNCCG

SEQ ID NO: 77 - protein, 450 aa, Physcomitrella patens
  MISC_FEATURE (1)..(450): CBL-interacting protein kinase (EST443)
MVMRKVGKYEVGRTIGEGTFAKVKFAQNTETGESVAMKVLDRQTVLKHKMVEQIRREISIMKLVRHPNVVRLHEVLASRCKIYIILEFVTGGELFDKIVHQGRLNENDSRKYFQQLMDGVDYCHSKGVSHRDLKPENLLLDSLDNLKISDFGLSALPQQVREDGLLHTTCGTPNYVAPEVLNDKGYDGAVADIWSCGVILFVLMAGFLPFDEADLNTLYSKIREADFTCPPWFSSGAKTLITNILDPNPLTRIRMRGIRDDEWFKKNYVPVRMYDDEDINLDDVETAFDDSKEQFVKEQREVKDVGPSLMNAFELISLSQGLNLSALFDRRQDHVKRQTRFTSKKPARDIINRMETAAKSMGFGVGTRNYKMRLEAASECRISQHLAVAIEVYEVAPSLFMIEVRKAAGDTLEYHKFYKSFCTRLKDIIWTTAVDKDEVKTLTPSVVKNK

SEQ ID NO: 78 - protein, 457 aa, Populus trichocarpa
  MISC_FEATURE (1)..(457): CBL-interacting protein kinase (ABJ91230)
MSSSRSGGSGSRTRVGRYELGRTLGEGTFAKVKFARNVETGENVAIKILDKEKVLKHKMIGQIKREISTMKLIRHPNVVRMYEVMASKTKIYIVLEFVTGGELFDKIASKGRLKEDEARKYFQQLINAVDYCHSRGVYHRDLKPENLLLDASGFLKVSDFGLSALPQQVREDGLLHTTCGTPNYVAPEVINNKGYDGAKADLWSCGVILFVLMAGYLPFEESNLMALYKKIFKADFTCPPWFSSSAKKLIKRILDPNPSTRITISELIENEWFKKGYKPPTFEKANVSLDDVDSIFNESMDSQNLVVERREEGFIGPMAPVTMNAFELISTSQGLNLSSLFEKQMGLVKRETRFTSKHSASEIISKIEAAAAPLGFDVKKNNFKMKLQGEKDGRKGRLSVSTEVFEVAPSLYMVEVRKSDGDTLEFHKFYKNLSTGLKDIVWKTIDEEEEEEAATNG

SEQ ID NO: 79 - protein, 466 aa, Populus trichocarpa
  MISC_FEATURE (1)..(466): CBL-interacting protein kinase (ABJ91231)
MSSSRSGGGGGGGGGGSGSKTRVGRYELGRTLGEGNFAKVKFARNVETKENVAIKILDKENVLKHKMIGQIKREISTMKLIRHPNVVRMYEVMASKTKIYIVLQFVTGGELFDKIASKGRLKEDEARKYFQQLICAVDYCHSRGVYHRDLKPENLLMDANGILKVSDFGLSALPQQVREDGLLHTTCGTPNYVAPEVINNKGYDGAKADLWSCGVILFVLMAGYLPFEEANLMALYKKIFKADFTCPPWFSSSAKKLIKRILDPNPSTRITIAELIENEWFKKGYKPPAFEQANVSLDDVNSIFNESVDSRNLVVERREEGFIGPMAPVTMNAFELISTSQGLNLSSLFEKQMGLVKRESRFTSKHSASEIISKIEAAAAPLGFDVKKNNFKMKLQGDKDGRKGRLSVATEIFEVAPSLYMVEVRKSGGDTLEFHKFYKNLSTGLKDIVWKTIDEEKEEEEAATNG

SEQ ID NO: 80 - protein, 450 aa, Oryza sativa
  MISC_FEATURE (1)..(450): CBL-interacting protein kinase (NP_001058901)
MSVSGGRTRVGRYELGRTLGEGTFAKVKFARNADSGENVAIKILDKDKVLKHKMIAQIKREISTMKLIRHPNVIRMHEVMASKTKIYIVMELVTGGELFDKIASRGRLKEDDARKYFQQLINAVDYCHSRGVYHRDLKPENLLLDASGTLKVSDFGLSALSQQVREDGLLHTTCGTPNYVAPEVINNKGYDGAKADLWSCGVILFVLMAGYLPFEDSNLMSLYKKIFKADFSCPSWFSTSAKKLIKKILDPNPSTRITIAELINNEWFKKGYQPPRFETADVNLDDINSIFNESGDQTQLVVERREERPSVMNAFELISTSQGLNLGTLFEKQSQGSVKRETRFASRLPANEILSKIEAAAGPMGFNVQKRNYKLKLQGENPGRKGQLAIATEVFEVTPSLYMVELRKSNGDTLEFHKFYHNISNGLKDVMWKPESSIIAGDEIQHRRSP

SEQ ID NO: 81 - protein, 447 aa, Arabidopsis thaliana
  MISC_FEATURE (1)..(447): CBL-interacting protein kinase (NP_171622)
MSGSRRKATPASRTRVGNYEMGRTLGEGSFAKVKYAKNTVTGDQAAIKILDREKVFRHKMVEQLKREISTMKLIKHPNVVEIIEVMASKTKIYIVLELVNGGELFDKIAQQGRLKEDEARRYFQQLINAVDYCHSRGVYHRDLKPENLILDANGVLKVSDFGLSAFSRQVREDGLLHTACGTPNYVAPEVLSDKGYDGAAADVWSCGVILFVLMAGYLPFDEPNLMTLYKRICKAEFSCPPWFSQGAKRVIKRILEPNPITRISIAELLEDEWFKKGYKPPSFDQDDEDITIDDVDAAFSNSKECLVTEKKEKPVSMNAFELISSSSEFSLENLFEKQAQLVKKETRFTSQRSASEIMSKMEETAKPLGFNVRKDNYKIKMKGDKSGRKGQLSVATEVFEVAPSLHVVELRKTGGDTLEFHKFYKNFSSGLKDVVWNTDAAAEEQKQ

SEQ ID NO: 82 - protein, 441 aa, Populus trichocarpa
  MISC_FEATURE (1)..(441): CBL-interacting protein kinase (ABJ91219)
MSVKVPAARTRVGKYELGKTIGEGSFAKVKVAKNVQTGDVVAIKILDRDQVLRHKMVEQLKREISTMKLIKHPNVIKIFEVMASKTKIYIVIEFVDGGELFDKIAKHGRLKEDEARRYFQQLIKAVDYCHSRGVFHRDLKPENLLLDSRGVLKVSDFGLSALSQQLRGDGLLHTACGTPNYVAPEVLRDQGYDGTASDVWSCGVILYVLMAGFLPFSESSLVVLYRKICRADFTFPSWFSSGAKKLIKRILDPKPLTRITVSEILEDEWFKKGYKPPQFEQEEDVNIDDVDAVFNDSKEHLVTERKVKPVSINAFELISKTQGFSLDNLFGKQAGVVKRETHIASHSPANEIMSRIEEAAKPLGFNVDKRNYKMKLKGDKSGRKGQLSVATEVFEVAPSLHMVELRKIGGDTLEFHKFYKSFSSGLKDVVWKSDQTIEGLR

SEQ ID NO: 83 - protein, 260 aa, Nicotiana tabacum
  MISC_FEATURE (1)..(260): 14-3-3 protein (BAD12177)
MAESTREENVYMAKLAEQAERYEEMVEFMEKVAKTVDVEELTVEERNLLSVAYKNVIGARRASWRIISSIEQKEESRGNEDHVSSIKEYRGKIEAELSKICDGILNLLESHLIPVASTAESKVFYLKMKGDYHRYLAEFKTGAERKEAAENTLLAYKSAQDIALAELAPTHPIRLGLALNFSVFYYEILNSSDRACNLAKQAFDDAIAELDTLGEESYKDSTLIMQLLRDNLTLWTSDSTDDAGDEIKEASKRESGDGEQ

SEQ ID NO: 84 - protein, 264 aa, Manihot esculenta
  MISC_FEATURE (1)..(264): 14-3-3 protein (AAY67798)
MLPTESSREENVYMAKLAEQAERYEEMVEFMEKVAKTVDVEELTVEERNLLSVAYKNVIGARRASWRIISSIEQKEESRGNEDHVSIIKEYRGKIEAELSKICDGILSLLESHLIPSASSAESKVFYLKMKGDYHRYLAEFKTAAERKEAAESTLLAYKSAQDIALADLAPTHPIRLGLALNFSVFYYEILNSPDRACNLAKQAFDEAISELDTLGEESYKDSTLIMQLLRDNLTLWTSDITDEAGDEIKDASKRESGEGQPQQ

SEQ ID NO: 85 - protein, 260 aa, Nicotiana tabacum
  MISC_FEATURE (1)..(260): 14-3-3 protein (BAD12176)
MAESTREENVYMAKLAEQAERYEEMVEFMEKVAKTVDVEELTVEERNLLSVAYKNVIGARRASWRIISSIEQKEESRGNEDHVSSIKEYRGKIEAELSKICDGILNLLESHLIPVASTAESKVFYLKMKGDYHRYLAEFKTGAERKEAAENTLLAYKSAQDIALAELAPTHPIRLGLALNFSVFYYEILNSSDRACNLAKQAFDDAIAELDTLGEESYKDSTLIMQLLRDNLTLWTSDTTDDAGDEIKEASKRESGEGEQ

SEQ ID NO: 86 - protein, 259 aa, Fritillaria agrestis
  MISC_FEATURE (1)..(259): 14-3-3 protein (AAC04811)
MSPAEPSREENVYMAKLAEQAERYEEMVEFMEKVARTVDTEELTVEERNLLSVAYKNVIGARRASWRIISSIEQKEESRGNEDHVALIKDYRGKIEAELSKICDGILKLLDSHLVPSSTAAESKVFYLKMKGDYHRYLAEFKSGAERKEAAESTLLAYKSAQDIALAELAPTHPIRLGLALNFSVFYYEILNSPDRACNLAKQAFDEAISELDTLGEESYKDSTLIMQLLRDNLTLWTSDINEEAGDEIKEASKAGEGQ

SEQ ID NO: 87 - protein, 259 aa, Lilium longiflorum
  MISC_FEATURE (1)..(259): 14-3-3 protein (Q9SP07)
MSPAEPSREENVYMAKLAEQAERYEEMVEFMEKVARTVDTEELTVEERNLLSVAYKNVIGARRASWRIISSIEQKEESRGNEDHVALIKDYRGKIEAELSKICDGILKLLDSHLVPSSTAPESKVFYLKMKGDYHRYLAEFKSGAERKEAAESTLLAYKSAQDIALAELAPTHPIRLGLALNFSVFYYEILNSPDRACNLAKQAFDEAISELDTLGEESYKDSTLIMQLLRDNLTLWTSDINEEAGDEIKEASKAVEGQ

SEQ ID NO: 88 - protein, 258 aa, Physcomitrella patens
  MISC_FEATURE (1)..(258): 14-3-3 protein (EST217)
MSTEKERESYVYMAKLAEQAERYDEMVESMKKVAKLDVELTVEERNLLSVGYKNVIGARRASWRIMSSIEQKEESKGNEQNVKRIKDYRHKVEEELSKICNDILSIIDGHLIPSSSTGESTVFYYKMKGDYYRYLAEFKTGNERKEAADQSLKAYQAASSTAVTDLAPTHPIRLGLALNFSVFYYEILNSPERACHLAKQAFDEAIAELDTLSEESYKDSTLIMQLLRDNLTLWTSDLQDEGGDDQGKGDDMRPEEAE

(19) United States
(12) Patent Application Publication
(10) Pub. No.: US 2010/0199388 A1
(43) Pub. Date: Aug. 5, 2010

(54) Transgenic Plants with Increased Stress Tolerance and Yield

(75) Inventors: Damian Allen, West Lafayette, IN (US); Amber Shirley, Durham, NC (US); Bryan D. McKersie, Raleigh, NC (US)

Correspondence Address: BASF CORPORATION, CARL-BOSCH-STRASSE 38, LUDWIGSHAFEN, D67056, DE

(73) Assignee: BASF Plant Science GmbH, Ludwigshafen (DE)

(21) Appl. No.: 12/601,531

(22) PCT Filed: May 28, 2008
(86) PCT No.: PCT/EP2008/056553

§ 371 Date: Nov. 24, 2009

Related U.S. Application Data
(60) Provisional application No. 60/932,147, filed on May 29, 2007.

(51) Int. Cl. A01H 1/00; A01H 5/00; C07H 21/04; C07K 14/00

(52) U.S. Cl. 800/290; 800/295; 536/23.1; 530/350

(57) ABSTRACT

Polynucleotides are disclosed which are capable of enhancing a growth, yield under water-limited conditions, and/or increased tolerance to an environmental stress of a plant transformed to contain such polynucleotides. Also provided are methods of using such polynucleotides and transgenic plants and agricultural products, including seeds, containing such polynucleotides as transgenes.

This application claims priority benefit of U.S. provisional patent application Ser. No. 60/932,147, filed May 29, 2007, the contents of which are hereby incorporated by reference.

### FIELD OF THE INVENTION

This invention relates generally to transgenic plants which overexpress nucleic acid sequences encoding polypeptides capable of conferring increased stress tolerance and consequently, increased plant growth and crop yield, under normal or abiotic stress conditions. Additionally, the invention relates to novel isolated nucleic acid sequences encoding polypeptides that confer upon a plant increased tolerance under abiotic stress conditions, and/or increased plant growth and/or increased yield under normal or abiotic stress conditions.

### BACKGROUND OF THE INVENTION

Abiotic environmental stresses, such as drought, salinity, heat, and cold, are major limiting factors of plant growth and crop yield. Crop yield is defined herein as the number of bushels of relevant agricultural product (such as grain, forage, or seed) harvested per acre. Crop losses and crop yield losses of major crops such as soybean, rice, maize (corn), cotton, and wheat caused by these stresses represent a significant economic and political factor and contribute to food shortages in many underdeveloped countries.

Water availability is an important aspect of the abiotic stresses and their effects on plant growth. Continuous exposure to drought conditions causes major alterations in the plant metabolism which ultimately lead to cell death and consequently to yield losses. Because high salt content in some soils results in less water being available for cell intake, high salt concentration has an effect on plants similar to the effect of drought on plants. Additionally, under freezing temperatures, plant cells lose water as a result of ice formation within the plant. Accordingly, crop damage from drought, heat, salinity, and cold stress, is predominantly due to dehydration.

Because plants are typically exposed to conditions of reduced water availability during their life cycle, most plants have evolved protective mechanisms against desiccation caused by abiotic stresses. However, if the severity and duration of dessication conditions are too great, the effects on development, growth, plant size, and yield of most crop plants are profound. Developing plants efficient in water use is therefore a strategy that has the potential to significantly improve human life on a worldwide scale.

Traditional plant breeding strategies are relatively slow and require abiotic stress-tolerant founder lines for crossing with other germplasm to develop new abiotic stress-resistant lines. Limited germplasm resources for such founder lines and incompatibility in crosses between distantly related plant species represent significant problems encountered in conventional breeding. Breeding for tolerance has been largely unsuccessful.

Many agricultural biotechnology companies have attempted to identify genes that could confer tolerance to abiotic stress responses, in an effort to develop transgenic abiotic stress-tolerant crop plants. Although some genes that are involved in stress responses or water use efficiency in plants have been characterized, the characterization and cloning of plant genes that confer stress tolerance and/or water use efficiency remains largely incomplete and fragmented. To date, success at developing transgenic abiotic stress-tolerant crop plants has been limited, and no such plants have been commercialized.

In order to develop transgenic abiotic stress-tolerant crop plants, it is necessary to assay a number of parameters in model plant systems, greenhouse studies of crop plants, and in field trials. For example, water use efficiency (WUE), is a parameter often correlated with drought tolerance. Studies of a plant's response to dessication, osmotic shock, and temperature extremes are also employed to determine the plant's tolerance or resistance to abiotic stresses. When testing for the impact of the presence of a transgene on a plant's stress tolerance, the ability to standardize soil properties, temperature, water and nutrient availability and light intensity is an intrinsic advantage of greenhouse or plant growth chamber environments compared to the field.

WUE has been defined and measured in multiple ways. One approach is to calculate the ratio of whole plant dry weight, to the weight of water consumed by the plant throughout its life. Another variation is to use a shorter time interval when biomass accumulation and water use are measured. Yet another approach is to use measurements from restricted parts of the plant, for example, measuring only aerial growth and water use. WUE also has been defined as the ratio of CO₂ uptake to water vapor loss from a leaf or portion of a leaf, often measured over a very short time period (e.g. seconds/minutes). The ratio of ¹³C/¹²C fixed in plant tissue, and measured with an isotope ratio mass-spectrometer, also has been used to estimate WUE in plants using C₃ photosynthesis.

An increase in WUE is informative about the relatively improved efficiency of growth and water consumption, but this information taken alone does not indicate whether one of these two processes has changed or both have changed. In selecting traits for improving crops, an increase in WUE due to a decrease in water use, without a change in growth would have particular merit in an irrigated agricultural system where the water input costs were high. An increase in WUE driven mainly by an increase in growth without a corresponding jump in water use would have applicability to all agricultural systems. In many agricultural systems where water supply is not limiting, an increase in growth, even if it came at the expense of an increase in water use (i.e. no change in WUE), could also increase yield. Therefore, new methods to increase both WUE and biomass accumulation are required to improve agricultural productivity.

Concomitant with measurements of parameters that correlate with abiotic stress tolerance are measurements of parameters that indicate the potential impact of a transgene on crop yield. For forage crops like alfalfa, silage corn, and hay, the plant biomass correlates with the total yield. For grain crops, however, other parameters have been used to estimate yield, such as plant size, as measured by total plant dry weight, above-ground dry weight, above-ground fresh weight, leaf area, stem volume, plant height, rosette diameter, leaf length, root length, root mass, tiller number, and leaf number. Plant size at an early developmental stage will typically correlate with plant size later in development. A larger plant with a greater leaf area can typically absorb more light and carbon dioxide than a smaller plant and therefore will likely gain a greater weight during the same period. This is in addition to the potential continuation of the micro-environmental or genetic advantage that the plant had to achieve the larger size initially. There is a strong genetic component to plant size and growth rate, and so for a range of diverse genotypes plant size under one environmental condition is likely to correlate with size under another. In this way a standard environment is used to approximate the diverse and dynamic environments encountered at different locations and times by crops in the field.

Harvest index, the ratio of seed yield to above-ground dry weight, is relatively stable under many environmental conditions and so a robust correlation between plant size and grain yield is possible. Plant size and grain yield are intrinsically linked, because the majority of grain biomass is dependent on current or stored photosynthetic productivity by the leaves and stem of the plant. Therefore, selecting for plant size, even at early stages of development, has been used as to screen for plants that may demonstrate increased yield when exposed to field testing. As with abiotic stress tolerance, measurements of plant size in early development, under standardized conditions in a growth chamber or greenhouse, are standard practices to measure potential yield advantages conferred by the presence of a transgene.

There is a need, therefore, to identify additional genes expressed in stress tolerant plants and/or plants that are efficient in water use that have the capacity to confer stress tolerance and/or increased water use efficiency to the host plant and to other plant species. Newly generated stress tolerant plants and/or plants with increased water use efficiency will have many advantages, such as an increased range in which the crop plants can be cultivated, by for example, decreasing the water requirements of a plant species. Other desirable advantages include increased resistance to lodging, the bending of shoots or stems in response to wind, rain, pests, or disease.

### SUMMARY OF THE INVENTION

The present inventors have discovered that transforming a plant with certain polynucleotides results in enhancement of the plant's growth and response to environmental stress, and accordingly the yield of the agricultural products of the plant is increased, when the polynucleotides are present in the plant as transgenes. The polynucleotides capable of mediating such enhancements have been isolated from Physcomitrella patens, Hordeum vulgare, Brassica napus, Linum usitatissimum, Orzya sativa, Helianthus annuus, Triticum aestivum, and Glycine max and are listed in Table 1, and the sequences thereof are set forth in the Sequence Listing as indicated in Table 1.

TABLE 1

| | | Polynucleotide | Amino acid |
|---|---|---|---|
| Gene ID | Organism | SEQ ID NO | SEQ ID NO |
| EST462 | P. patens | 1 | 2 |
| EST329 | P. patens | 3 | 4 |
| EST373 | P. patens | 5 | 6 |
| HV62561245 | H. vulgare | 7 | 8 |
| BN43173847 | B. napus | 9 | 10 |
| BN46735603 | B. napus | 11 | 12 |
| GM52504443 | G. max | 13 | 14 |
| GM47122590 | G. max | 15 | 16 |
| GM52750153 | G. max | 17 | 18 |
| EST548 | P. patens | 19 | 20 |
| GM50181682 | G. max | 21 | 22 |
| HV62638446 | H. vulgare | 23 | 24 |
| TA56528531 | T. aestivum | 25 | 26 |
| HV62624858 | H. vulgare | 27 | 28 |
| LU61640267 | L. usitatissimum | 29 | 30 |
| LU61872929 | L. usitatissimum | 31 | 32 |
| LU61896092 | L. usitatissimum | 33 | 34 |
| LU61748785 | L. usitatissimum | 35 | 36 |
| OS34706416 | O. sativa | 37 | 38 |
| GM49750953 | G. max | 39 | 40 |
| HA66696606 | H. annuus | 41 | 42 |
| HA66783477 | H. annuus | 43 | 44 |
| HA66705690 | H. annuus | 45 | 46 |
| TA59921546 | T. aestivum | 47 | 48 |
| HV62657638 | H. vulgare | 49 | 50 |
| BN43540204 | B. napus | 51 | 52 |
| BN45139744 | B. napus | 53 | 54 |
| BN43613585 | B. napus | 55 | 56 |
| LU61965240 | L. usitatissimum | 57 | 58 |
| LU62294414 | L. usitatissimum | 59 | 60 |
| LU61723544 | L. usitatissimum | 61 | 62 |
| LU61871078 | L. usitatissimum | 63 | 64 |
| LU61569070 | L. usitatissimum | 65 | 66 |
| OS34999273 | O. sativa | 67 | 68 |
| HA66779896 | H. annuus | 69 | 70 |
| OS32667913 | O. sativa | 71 | 72 |
| HA66453181 | H. annuus | 73 | 74 |
| HA66709897 | H. annuus | 75 | 76 |

In one embodiment, the invention provides a transgenic plant transformed with an expression cassette comprising an isolated polynucleotide encoding a CBL-interacting protein kinase having a sequence as set forth in SEQ ID NO:2.

In another embodiment, the invention provides a transgenic plant transformed with an expression cassette comprising an isolated polynucleotide encoding a 14-3-3 protein having a sequence as set forth in SEQ ID NO:4.

In another embodiment, the invention provides a transgenic plant transformed with an expression cassette comprising an isolated polynucleotide encoding a RING H2 zinc finger protein or a RING H2 zinc finger protein domain.

In another embodiment, the invention provides a transgenic plant transformed with an expression cassette comprising an isolated polynucleotide encoding a GTP binding protein or a GTP binding protein domain.

In a further embodiment, the invention provides a seed produced by the transgenic plant of the invention, wherein the seed is true breeding for a transgene comprising the polynucleotide described above. Plants derived from the seed of the invention demonstrate increased tolerance to an environmental stress, and/or increased plant growth, and/or increased yield, under normal or stress conditions as compared to a wild type variety of the plant.

In a still another aspect, the invention provides products produced by or from the transgenic plants of the invention, their plant parts, or their seeds, such as a foodstuff, feedstuff, food supplement, feed supplement, cosmetic or pharmaceutical.

The invention further provides the isolated polynucleotides identified in Table 1 below, and isolated polypeptides identified in Table 1. The invention is also embodied in recombinant vector comprising an isolated polynucleotide of the invention.

In yet another embodiment, the invention concerns a method of producing the aforesaid transgenic plant, wherein the method comprises transforming a plant cell with an expression vector comprising an isolated polynucleotide of the invention, and generating from the plant cell a transgenic plant that expresses the polypeptide encoded by thepolynucleotide. Expression of the polypeptide in the plant results in increased tolerance to an environmental stress, and/or growth, and/or yield under normal or stress conditions as compared to a wild type variety of the plant.

In still another embodiment, the invention provides a method of increasing a plant's tolerance to an environmental stress, and/or growth, and/or yield. The method comprises the steps of transforming a plant cell with an expression cassette comprising an isolated polynucleotide of the invention, and generating a transgenic plant from the plant cell, wherein the transgenic plant comprises the polynucleotide.

### BRIEF DESCRIPTION OF THE DRAWINGS

FIG. 1 is an alignment of EST462 of P. patens with the known CBL-interacting protein kinases identified in Table 2.

FIG. 2 is an alignment of EST329 of P. patens with the known 14-3-3 proteins identified in Table 3.

FIG. 3 is an alignment of EST373 with the known RING H2 zinc finger proteins identified in Table 4.

FIGS. 4A and 4B contain an alignment of EST548 with the known GTP binding proteins identified in Table 5.

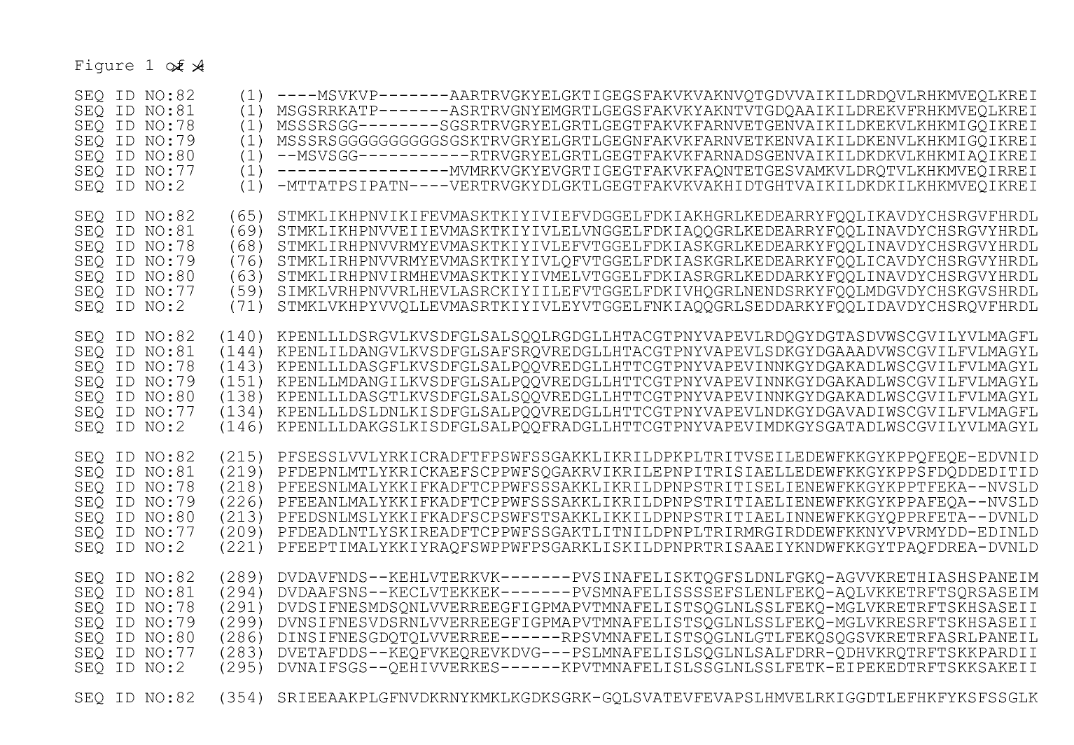

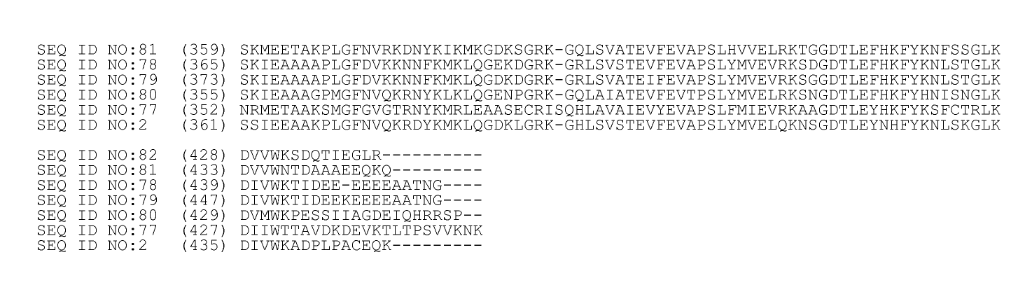

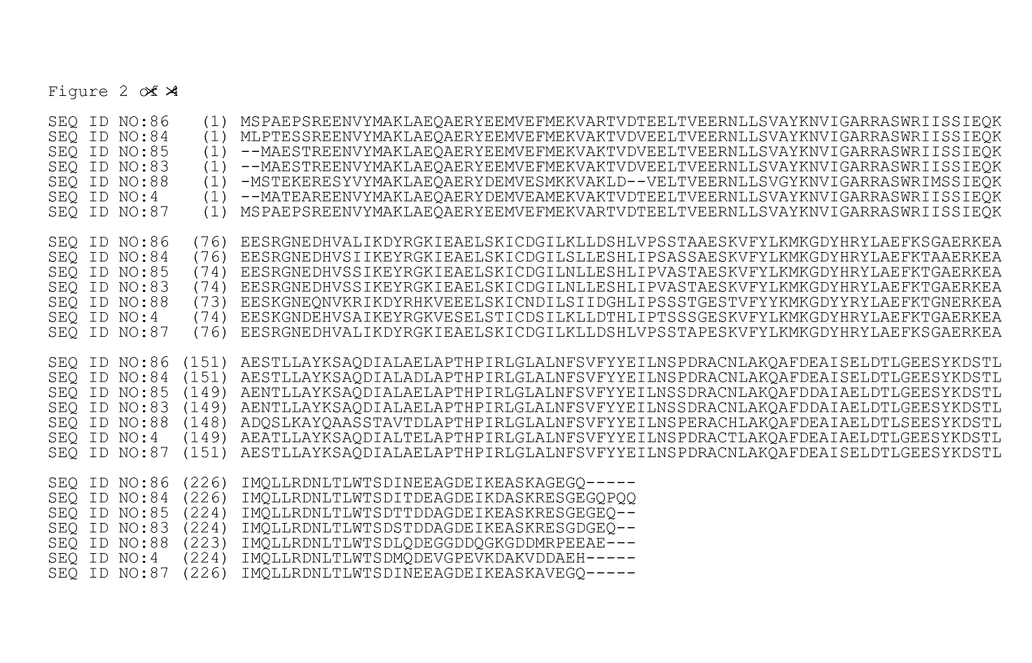

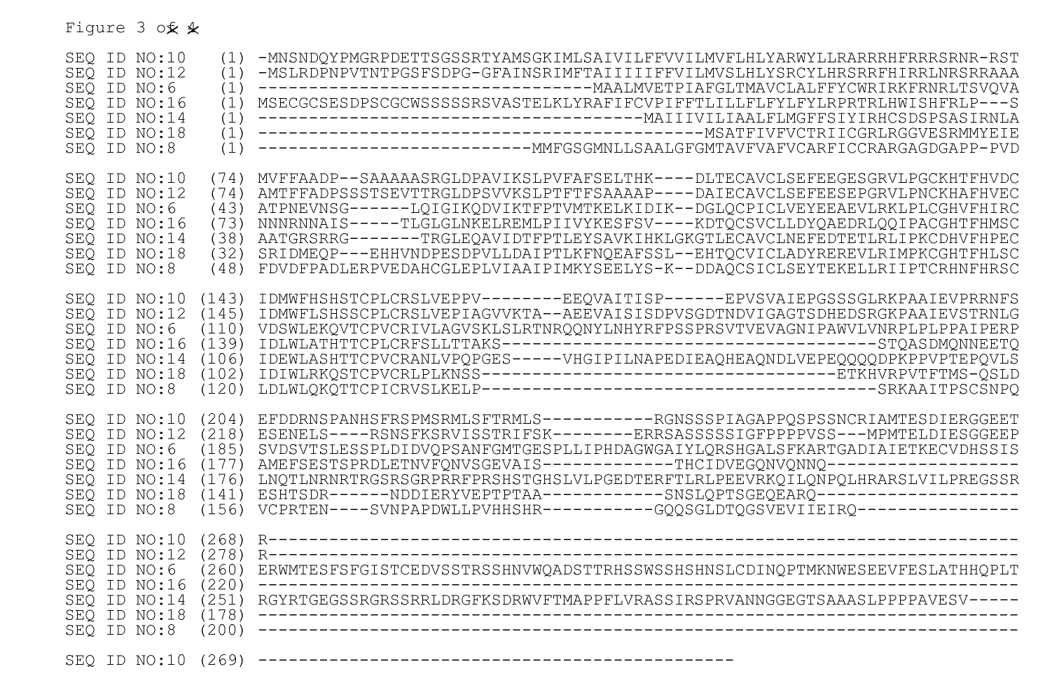

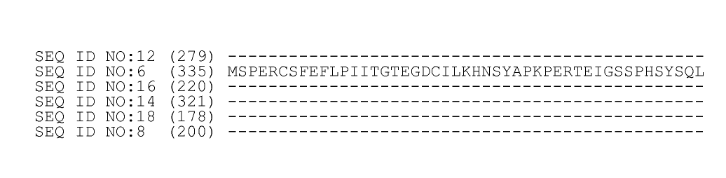

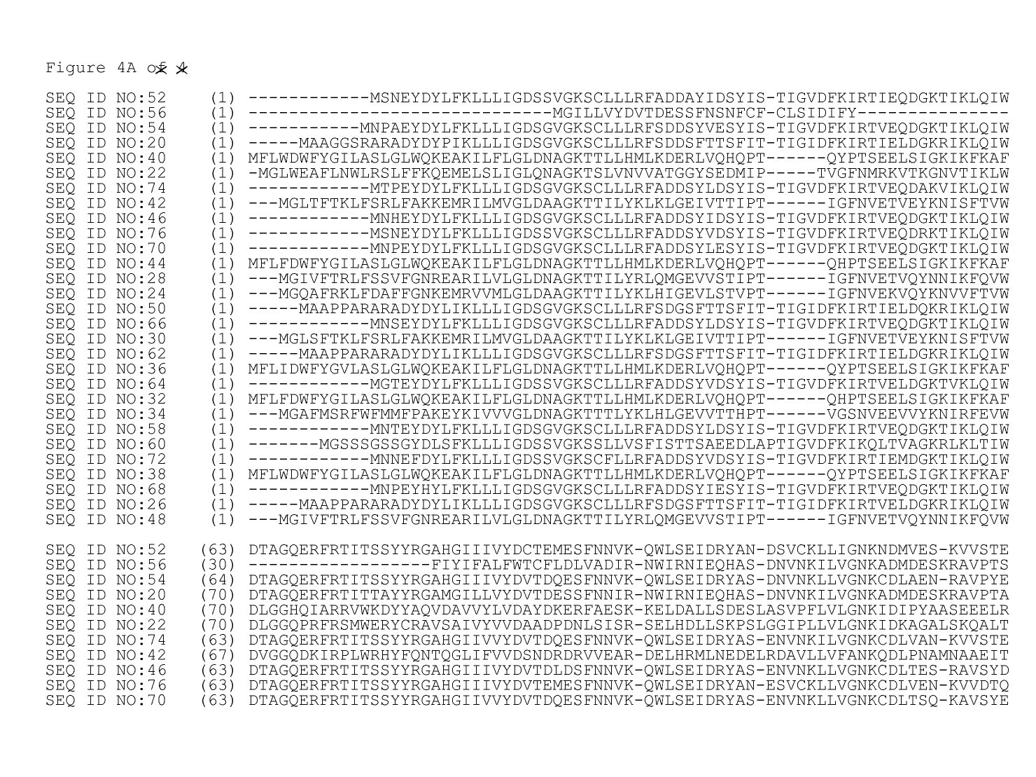

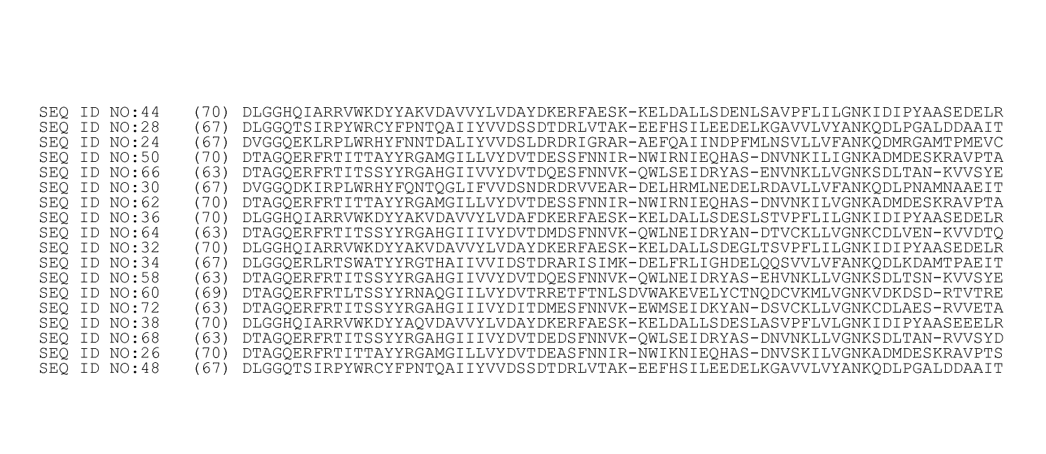

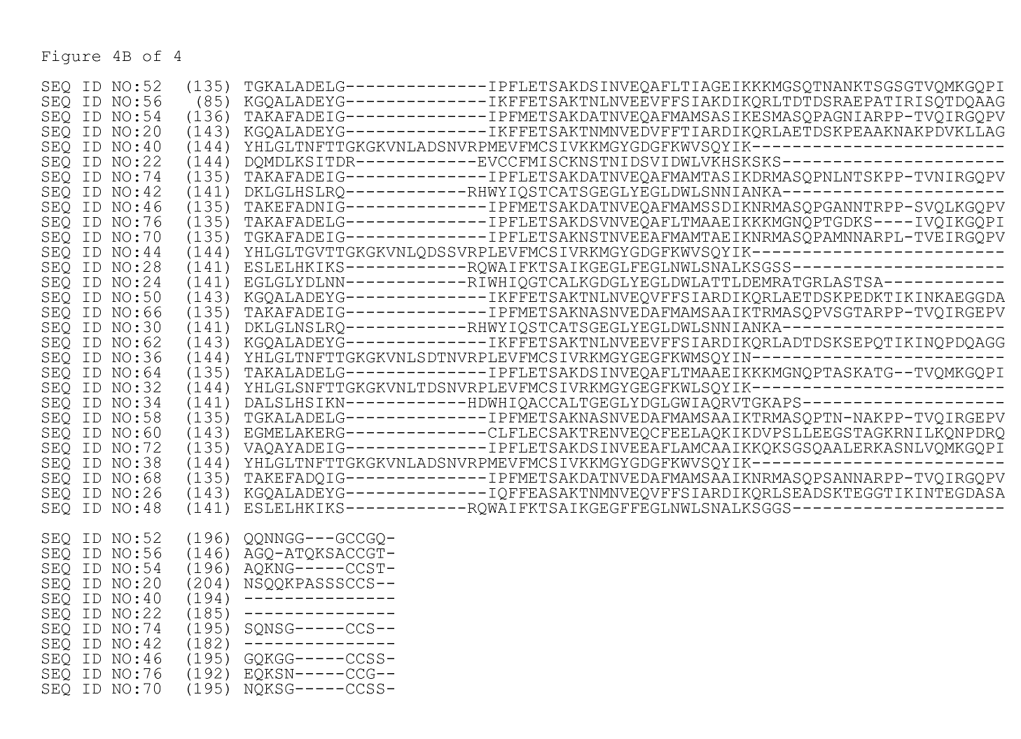

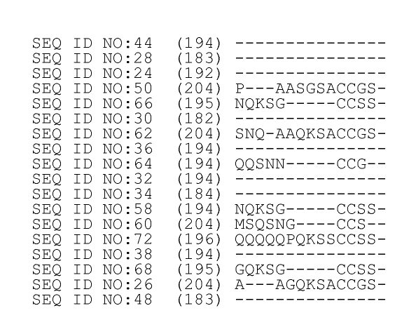

### DETAILED DESCRIPTION OF THE PREFERRED EMBODIMENTS

Throughout this application, various publications are referenced. The disclosures of all of these publications and those references cited within those publications in their entireties are hereby incorporated by reference into this application in order to more fully describe the state of the art to which this invention pertains. The terminology used herein is for the purpose of describing specific embodiments only and is not intended to be limiting. As used herein, “a” or “an” can mean one or more, depending upon the context in which it is used. Thus, for example, reference to “a cell” can mean that at least one cell can be used.

In one embodiment, the invention provides a transgenic plant that overexpresses an isolated polynucleotide identified in Table 1, or a homolog thereof. The transgenic plant of the invention demonstrates an increased tolerance to an environmental stress as compared to a wild type variety of the plant. The overexpression of such isolated nucleic acids in the plant may optionally result in an increase in plant growth or in yield of associated agricultural products, under normal or stress conditions, as compared to a wild type variety of the plant. Without wishing to be bound by any theory, the increased tolerance to an environmental stress, increased growth, and/or increased yield of a transgenic plant of the invention is believed to result from an increase in water use efficiency of the plant.

As defined herein, a “transgenic plant” is a plant that has been altered using recombinant DNA technology to contain an isolated nucleic acid which would otherwise not be present in the plant. As used herein, the term “plant” includes a whole plant, plant cells, and plant parts. Plant parts include, but are not limited to, stems, roots, ovules, stamens, leaves, embryos, meristematic regions, callus tissue, gametophytes, sporophytes, pollen, microspores, and the like. The transgenic plant of the invention may be male sterile or male fertile, and may further include transgenes other than those that comprise the isolated polynucleotides described herein.

As used herein, the term “variety” refers to a group of plants within a species that share constant characteristics that separate them from the typical form and from other possible varieties within that species. While possessing at least one distinctive trait, a variety is also characterized by some variation between individuals within the variety, based primarily on the Mendelian segregation of traits among the progeny of succeeding generations. A variety is considered “true breeding” for a particular trait if it is genetically homozygous for that trait to the extent that, when the true-breeding variety is self-pollinated, a significant amount of independent segregation of the trait among the progeny is not observed. In the present invention, the trait arises from the transgenic expression of one or more isolated polynucleotides introduced into a plant variety. As also used herein, the term “wild type variety” refers to a group of plants that are analyzed for comparative purposes as a control plant, wherein the wild type variety plant is identical to the transgenic plant (plant transformed with an isolated polynucleotide in accordance with the invention) with the exception that the wild type variety plant has not been transformed with an isolated polynucleotide in accordance with the invention.

As defined herein, the term “nucleic acid” and “polynucleotide” are interchangeable and refer to RNA or DNA that is linear or branched, single or double stranded, or a hybrid thereof. The term also encompasses RNA/DNA hybrids. An “isolated” nucleic acid molecule is one that is substantially separated from other nucleic acid molecules which are present in the natural source of the nucleic acid (i.e., sequences encoding other polypeptides). For example, a cloned nucleic acid is considered isolated. A nucleic acid is also considered isolated if it has been altered by human intervention, or placed in a locus or location that is not its natural site, or if it is introduced into a cell by transformation. Moreover, an isolated nucleic acid molecule, such as a cDNA molecule, can be free from some of the other cellular material with which it is naturally associated, or culture medium when produced by recombinant techniques, or chemical precursors or other chemicals when chemically synthesized. While it may optionally encompass untranslated sequence located at both the 3′ and 5′ ends of the coding region of a gene, an isolated nucleic acid is preferably free of the sequences which naturally flank the coding region in its naturally occurring replicon.

As used herein, the term “environmental stress” refers to a sub-optimal condition associated with salinity, drought, nitrogen, temperature, metal, chemical, pathogenic, or oxidative stresses, or any combination thereof. The terms “water use efficiency” and “WUE” refer to the amount of organic matter produced by a plant divided by the amount of water used by the plant in producing it, i.e., the dry weight of a plant in relation to the plant's water use. As used herein, the term “dry weight” refers to everything in the plant other than water, and includes, for example, carbohydrates, proteins, oils, and mineral nutrients.

Any plant species may be transformed to create a transgenic plant in accordance with the invention. The transgenic plant of the invention may be a dicotyledonous plant or a monocotyledonous plant. For example and without limitation, transgenic plants of the invention may be derived from any of the following diclotyledonous plant families: Leguminosae, including plants such as pea, alfalfa and soybean; Umbelliferae, including plants such as carrot and celery; Solanaceae, including the plants such as tomato, potato, aubergine, tobacco, and pepper; Cruciferae, particularly the genus Brassica, which includes plant such as oilseed rape, beet, cabbage, cauliflower and broccoli); and Arabidopsis thaliana; Compositae, which includes plants such as lettuce; Malvaceae, which includes cotton; Fabaceae, which includes plants such as peanut, and the like. Transgenic plants of the invention may be derived from monocotyledonous plants, such as, for example, wheat, barley, sorghum, millet, rye, triticale, maize, rice, oats, switchgrass, miscanthus and sugarcane. Transgenic plants of the invention are also embodied as trees such as apple, pear, quince, plum, cherry, peach, nectarine, apricot, papaya, mango, and other woody species including coniferous and deciduous trees such as poplar, pine, sequoia, cedar, oak, willow, and the like. Especially preferred are Arabidopsis thaliana, Nicotiana tabacum, oilseed rape, soybean, corn (maize), wheat, linseed, potato and tagetes.

As shown in Table 1, one embodiment of the invention is a transgenic plant transformed with an expression cassette comprising an isolated polynucleotide encoding a CBL-interacting protein kinase. The calcineurin B-like protein interacting protein kinase (CIPK) family of proteins represents a family of calcium dependent serine-threonine protein kinases. CIPKs have a two-domain structure consisting of a highly conserved N-terminal catalytic kinase domain and a less conserved C-terminal domain. It is this C-terminal domain that interacts with calcineurin B-like proteins (CBLs). The CIPK and CBL proteins interact directly in a calcium dependent manner to form a complex, which provides a regulatory mechanism for translating cellular calcium signals. A class of CIPKs has been identified distinguished by containing a minimum 24 amino acid protein interaction module that is both necessary and sufficient to mediate the interaction of CIPK and CBL proteins. This motif has been designated the NAF domain because of the characteristic asparagine, alanine, and phenylalanine residues it contains. An additional layer of regulation has been proposed for the NAF containing CIPK proteins by calcium dependent reversible membrane association following myristylation. These CIPKs have been demonstrated to be involved in plant stress signalling. Specifically, the SOS3(CBL4)/SOS2(CIPK24) signaling complex has been shown specifically to mediate salt stress signaling in Arabidopsis by regulating the membrane localized Na+/H+ exchanger SOS1.

The transgenic plant of this embodiment may comprise any polynucleotide encoding a CBL-interacting protein kinase having a sequence comprising amino acids 1 to 449 of SEQ ID NO:2. The transgenic plant of this embodiment may comprise a polynucleotide encoding a CBL-interacting protein kinase domain having a sequence comprising amino acids 21 to 293 of SEQ ID NO:2 or a NAF domain having a sequence comprising amino acids 315 to 376 of SEQ ID NO:2.

In another embodiment, the invention provides a transgenic plant transformed with an expression cassette comprising an isolated polynucleotide encoding a 14-3-3 protein. The 14-3-3 family of proteins form highly conserved dimeric proteins. They bind a diverse set of cellular proteins, over 200 of which are known to date. The structure of each monomer of 14-3-3 proteins consists of nine alpha helicies arranged in an antiparallel bundle creating a groove, which binds a phosphorylated ligand. The 14-3-3 proteins themselves can also be regulated by phosphorylation, dimerization, cAMP, and Ca⁺⁺ ions. The dimeric form of 14-3-3 proteins can accommodate two ligands, one in each groove of the monomer; thereby, 14-3-3 proteins play a role in scaffolding diverse protein targets and modifying the structure of individual protein targets. Binding of 14-3-3 proteins has been demonstrated to alter enzymes in a reversible manner, activation or inactivation, and can alter proteins via stabilization or degradation.

14-3-3 proteins have a highly conserved central domain, and variable N- and C-termini. It has been proposed that the C-terminal regions form a moveable cap that might regulate entry and exit of ligands from the central binding grooves and/or regulate specific binding of target ligands. Structural and truncated protein studies indicate that the C-terminal region has an inhibitory role and may prevent inappropriate interactions with 14-3-3 proteins and ligands by competing for binding within the groove.

The transgenic plant of this embodiment may comprise any polynucleotide encoding the 14-3-3 protein having the sequence comprising amino acids 1 to 257 of SEQ ID NO:4. The transgenic plant of this embodiment may comprise a polynucleotide encoding a 14-3-3 protein domain having a sequence comprising amino acids 6 to 243 of SEQ ID NO:4 or a C-terminal functional domain having a sequence comprising amino acids 245 to 258 of SEQ ID NO:4

As shown in Table 1, one embodiment of the invention is a transgenic plant transformed with an expression cassette comprising a polynucleotide encoding a RING H2 zinc finger protein or a RING H2 zinc finger protein domain. One of the regulators of protein degradation via the ubiquitin/26S proteasome pathway in Eukaryotes is ubiquitin ligases, also referred to as E3 enzymes. These E3 enzymes are responsible for recruiting the proteins that will be targeted for ubiquitination and thus act as the major substrate for the recognition component of the ubiquitination pathway. E3 ligases are grouped into 3 classes based upon the presence of a conserved domain. The RING type of E3 ligases can further be subdivided into simple and complex types. The simple type contains both the substrate-binding domain and the E2 binding RING domain in a single protein. The RING domain is similar to the zinc finger domain in containing cysteine and/or histidine to co-ordinate two zinc ions, but unlike a zinc finger, the RING domain functions as a protein-protein interaction domain. The canonical RING motif contains seven cysteines and one histidine. A family of C₃H₂C3/RING-H2 E3 ligases contains a substitution of the fifth cysteine for histidine. In Arabidopsis, this family of RING-H2 ligases has some evidence of being involved in growth regulator response, response to biotic stress, and plant development based upon elicitor and mutant studies.

The transgenic plant of this embodiment may comprise any polynucleotide encoding a RING H2 zinc finger protein. Preferably, the transgenic plant of this embodiment comprises a polynucleotide encoding a zinc finger, C3HC4 type domain having a sequence comprising amino acids 88 to 129 of SEQ ID NO:6; amino acids 98 to 139 of SEQ ID NO: 8; amino acids 121 to 162 of SEQ ID NO: 10; amino acids 123 to 164 of SEQ ID NO: 12; amino acids 84 to 125 of SEQ ID NO: 14; amino acids 117 to 158 of SEQ ID NO: 16; amino acids 80 to 121 of SEQ ID NO: 18. More preferably, the transgenic plant of this embodiment comprises a polynucleotide encoding a RING H2 zinc finger protein having a sequence comprising amino acids 1 to 381 of SEQ ID NO:6; amino aicds 1 to 199 of SEQ ID NO: 8; amino acids 1 to 268 of SEQ ID NO: 10; amino acids 1 to 278 of SEQ ID NO: 12; amino acids 1 to 320 of SEQ ID NO: 14; amino acids 1 to 219 of SEQ ID NO: 16; amino acids 1 to 177 of SEQ ID NO: 18.

In another embodiment, the invention provides a transgenic plant transformed with an expression cassette comprising an isolated polynucleotide encoding a GTP binding protein or a GTP binding protein domain. Monomeric/small G-proteins are involved in many different cellular processes and have been implicated in vesicle traffic/transport systems, cell cycle regulation, and protein import into organelles. When bound to a GTP nucleotide, GTP proteins activate cellular processes and become inactive when GTP is hydrolyzed to GDP. These proteins may be classified into five superfamilies based on structural and functional similarities: Ras, Rho/Rac/Cda42, Rab, Sar1/Arf, and Ran. Generally, members of only the Sar1 and Rab families of small G proteins are involved in vesicle trafficking in yeast and mammalian cells. In plants, Rab G proteins have been shown to function in a manner similar to their yeast and mammalian counterparts. Rab G proteins regulate endocytic trafficking pathways and biosynthetic trafficking pathways.

The transgenic plant of this embodiment may comprise any polynucleotide encoding a GTP binding protein. Preferably, the transgenic plant of this embodiment comprises a polynucleotide encoding a Ras family domain having a sequence comprising amino acids 17 to 179 of SEQ ID NO:20; amino acids 21 to 182 of SEQ ID NO: 22; amino acids 19 to 179 of SEQ ID NO: 24; amino acids 17 to 179 of SEQ ID NO: 26; amino acids 19 to 179 of SEQ ID NO: 28; amino acids 19 to 179 of SEQ ID NO: 30; amino aics 22 to 193 of SEQ ID NO: 32; amino acids 19 to 179 of SEQ ID NO: 34; amino acids 22 to 193 of SEQ ID NO: 36; amino acids 22 to 193 of SEQ ID NO: 38; amino acids 22 to 193 of SEQ ID NO: 40; amino acids 19 to 179 of SEQ ID NO: 42; amino acids 22 to 193 of SEQ ID NO: 44; amino acids 10 to 171 of SEQ ID NO: 46; amino acids 19 to 179 of SEQ ID NO: 48; amino acids 17 to 179 of SEQ ID NO: 50; amino acids 10 to 171 of SEQ ID NO: 52; amino acids 11 to 172 of SEQ ID NO: 54; amino acids 1 to 137 of SEQ ID NO: 56; amino acids 10 to 171 of SEQ ID NO: 58; amino acids 15 to 179 of SEQ ID NO: 60; amino aicds 17 to 195 of SEQ ID NO: 62; amino acids 10 to 171 of SEQ ID NO: 64; amino acids 10 to 171 of SEQ ID NO: 66; amino acids 10 to 171 of SEQ ID NO: 68; amino acids 10 to 171 of SEQ ID NO: 70, amino acids 10 to 171 of SEQ ID NO: 72; amino acids 10 to 171 of SEQ ID NO 74; amino acids 10 to 171 of SEQ ID NO: 76. More preferably, the transgenic plant of this embodiment comprises a polynucleotide encoding a GTP binding protein having a sequence comprising amino acids 1 to 216 of SEQ ID NO:20; amino acids 1 to 184 of SEQ ID NO: 22; amino acids 1 to 191 of SEQ ID NO: 24; amino acids 1 to 214 of SEQ ID NO: 26; amino acids 1 to 182 of SEQ ID NO: 28; amino acids 1 to 181 of SEQ ID NO: 30, amino acids 1 to 193 of SEQ ID NO: 32; amino acids 1 to 183 of SEQ ID NO: 34; amino acids 1 to 193 of SEQ ID NO: 36; amino acids 1 to 193 of SEQ ID NO: 38; amino acids 1 to 193 of SEQ ID NO: 40; amino acids 1 to 181 of SEQ ID NO: 42; amino acids 1 to 193 of SEQ ID NO: 44; amino acids 1 to 204 of SEQ ID NO: 46; amino acids 1 to 182 of SEQ ID NO: 48; amino acids 1 to 214 of SEQ ID NO: 50; amino acids 1 to 206 of SEQ ID NO: 52; amino acids 1 to 204 of SEQ ID NO: 54; amino acids 1 to 158 of SEQ ID NO: 56; amino acids 1 to 202 of SEQ ID NO: 58; amino acids 1 to 212 of SEQ ID NO: 60; amino acids 1 to 216 of SEQ ID NO: 62; amino acids 1 to 201 of SEQ ID NO: 64; amino acids 1 to 203 of SEQ ID NO: 66; amino acids 1 to 203 of SEQ ID NO: 68; amino acids 1 to 203 of SEQ ID NO: 70; amino acids 1 to 209 of SEQ ID NO: 72; amino acids 1 to 202 of SEQ ID NO: 74; amino acids 1 to 199 of SEQ ID NO: 76.

The invention further provides a seed produced by a transgenic plant expressing polynucleotide listed in Table 1, wherein the seed contains the polynucleotide, and wherein the plant is true breeding for increased growth and/or yield under normal or stress conditions and/or increased tolerance to an environmental stress as compared to a wild type variety of the plant. The invention also provides a product produced by or from the transgenic plants expressing the polynucleotide, their plant parts, or their seeds. The product can be obtained using various methods well known in the art. As used herein, the word “product” includes, but not limited to, a foodstuff, feedstuff, a food supplement, feed supplement, cosmetic or pharmaceutical. Foodstuffs are regarded as compositions used for nutrition or for supplementing nutrition. Animal feedstuffs and animal feed supplements, in particular, are regarded as foodstuffs. The invention further provides an agricultural product produced by any of the transgenic plants, plant parts, and plant seeds. Agricultural products include, but are not limited to, plant extracts, proteins, amino acids, carbohydrates, fats, oils, polymers, vitamins, and the like.

In a preferred embodiment, an isolated polynucleotide of the invention comprises a polynucleotide having a sequence selected from the group consisting of the nucleotide sequences listed in Table 1. These polynucleotides may comprise sequences of the coding region, as well as 5′ untranslated sequences and 3′ untranslated sequences.

A polynucleotide of the invention can be isolated using standard molecular biology techniques and the sequence information provided herein. For example, P. patens cDNAs of the invention were isolated from a P. patens library using a portion of the sequence disclosed herein. Synthetic oligonucleotide primers for polymerase chain reaction amplification can be designed based upon the nucleotide sequence shown in Table 1. A nucleic acid molecule of the invention can be amplified using cDNA or, alternatively, genomic DNA, as a template and appropriate oligonucleotide primers according to standard PCR amplification techniques. The nucleic acid molecule so amplified can be cloned into an appropriate vector and characterized by DNA sequence analysis. Furthermore, oligonucleotides corresponding to the nucleotide sequences listed in Table 1 can be prepared by standard synthetic techniques, e.g., using an automated DNA synthesizer.

“Homologs” are defined herein as two nucleic acids or polypeptides that have similar, or substantially identical, nucleotide or amino acid sequences, respectively. Homologs include allelic variants, analogs, and orthologs, as defined below. As used herein, the term “analogs” refers to two nucleic acids that have the same or similar function, but that have evolved separately in unrelated organisms. As used herein, the term “orthologs” refers to two nucleic acids from different species, but that have evolved from a common ancestral gene by speciation. The term homolog further encompasses nucleic acid molecules that differ from one of the nucleotide sequences shown in Table 1 due to degeneracy of the genetic code and thus encode the same polypeptide. As used herein, a “naturally occurring” nucleic acid molecule refers to an RNA or DNA molecule having a nucleotide sequence that occurs in nature (e.g., encodes a natural polypeptide).

To determine the percent sequence identity of two amino acid sequences (e.g., one of the polypeptide sequences of Table 1 and a homolog thereof), the sequences are aligned for optimal comparison purposes (e.g., gaps can be introduced in the sequence of one polypeptide for optimal alignment with the other polypeptide or nucleic acid). The amino acid residues at corresponding amino acid positions are then compared. When a position in one sequence is occupied by the same amino acid residue as the corresponding position in the other sequence then the molecules are identical at that position. The same type of comparison can be made between two nucleic acid sequences.

Preferably, the isolated amino acid homologs, analogs, and orthologs of the polypeptides of the present invention are at least about 50-60%, preferably at least about 60-70%, and more preferably at least about 70-75%, 75-80%, 80-85%, 85-90%, or 90-95%, and most preferably at least about 96%, 97%, 98%, 99%, or more identical to an entire amino acid sequence identified in Table 1. In another preferred embodiment, an isolated nucleic acid homolog of the invention comprises a nucleotide sequence which is at least about 40-60%, preferably at least about 60-70%, more preferably at least about 70-75%, 75-80%, 80-85%, 85-90%, or 90-95%, and even more preferably at least about 95%, 96%, 97%, 98%, 99%, or more identical to a nucleotide sequence shown in Table 1.

For the purposes of the invention, the percent sequence identity between two nucleic acid or polypeptide sequences is determined using Align 2.0 (Myers and Miller, CABIOS (1989) 4:11-17) with all parameters set to the default settings or the Vector NTI 9.0 (PC) software package (Invitrogen, 1600 Faraday Ave., Carlsbad, Calif. 92008). For percent identity calculated with Vector NTI, a gap opening penalty of 15 and a gap extension penalty of 6.66 are used for determining the percent identity of two nucleic acids. A gap opening penalty of 10 and a gap extension penalty of 0.1 are used for determining the percent identity of two polypeptides. All other parameters are set at the default settings. For purposes of a multiple alignment (Clustal W algorithm), the gap opening penalty is 10, and the gap extension penalty is 0.05 with blosum62 matrix. It is to be understood that for the purposes of determining sequence identity when comparing a DNA sequence to an RNA sequence, a thymidine nucleotide is equivalent to a uracil nucleotide.

Nucleic acid molecules corresponding to homologs, analogs, and orthologs of the polypeptides listed in Table 1 can be isolated based on their identity to said polypeptides, using the polynucleotides encoding the respective polypeptides or primers based thereon, as hybridization probes according to standard hybridization techniques under stringent hybridization conditions. As used herein with regard to hybridization for DNA to a DNA blot, the term “stringent conditions” refers to hybridization overnight at 60° C. in 10×Denhart's solution, 6×SSC, 0.5% SDS, and 100 μg/ml denatured salmon sperm DNA. Blots are washed sequentially at 62° C. for 30 minutes each time in 3×SSC/0.1% SDS, followed by 1×SSC/0.1% SDS, and finally 0.1×SSC/0.1% SDS. As also used herein, in a preferred embodiment, the phrase “stringent conditions” refers to hybridization in a 6×SSC solution at 65° C. In another embodiment, “highly stringent conditions” refers to hybridization overnight at 65° C. in 10×Denhart's solution, 6×SSC, 0.5% SDS and 100 μg/ml denatured salmon sperm DNA. Blots are washed sequentially at 65° C. for 30 minutes each time in 3×SSC/0.1% SDS, followed by 1×SSC/0.1% SDS, and finally 0.1×SSC/0.1% SDS. Methods for nucleic acid hybridizations are described in Meinkoth and Wahl, 1984, Anal. Biochem. 138:267-284; well known in the art (see, for example, Current Protocols in Molecular Biology, Chapter 2, Ausubel et al., eds., Greene Publishing and Wiley-Interscience, New York, 1995; and Tijssen, 1993, Laboratory Techniques in Biochemistry and Molecular Biology: Hybridization with Nucleic Acid Probes, Part I, Chapter 2, Elsevier, New York, 1993). Preferably, an isolated nucleic acid molecule of the invention that hybridizes under stringent or highly stringent conditions to a nucleotide sequence listed in Table 1 corresponds to a naturally occurring nucleic acid molecule.

There are a variety of methods that can be used to produce libraries of potential homologs from a degenerate oligonucleotide sequence. Chemical synthesis of a degenerate gene sequence can be performed in an automatic DNA synthesizer, and the synthetic gene is then ligated into an appropriate expression vector. Use of a degenerate set of genes allows for the provision, in one mixture, of all of the sequences encoding the desired set of potential sequences. Methods for synthesizing degenerate oligonucleotides are known in the art (See, e.g., Narang, 1983, Tetrahedron 39:3; Itakura et al., 1984, Annu. Rev. Biochem. 53:323; Itakura et al., 1984, Science 198:1056; Ike et al., 1983, Nucleic Acid Res. 11:477).

Additionally, optimized nucleic acids can be created. Preferably, an optimized nucleic acid encodes a polypeptide that has a function similar to those of the polypeptides listed in Table 1 and/or modulates a plant's growth and/or yield under normal or water-limited conditions and/or tolerance to an environmental stress, and more preferably increases a plant's growth and/or yield under normal or water-limited conditions and/or tolerance to an environmental stress upon its overexpression in the plant. As used herein, “optimized” refers to a nucleic acid that is genetically engineered to increase its expression in a given plant or animal. To provide plant optimized nucleic acids, the DNA sequence of the gene can be modified to: 1) comprise codons preferred by highly expressed plant genes; 2) comprise an A+T content in nucleotide base composition to that substantially found in plants; 3) form a plant initiation sequence; 4) to eliminate sequences that cause destabilization, inappropriate polyadenylation, degradation and termination of RNA, or that form secondary structure hairpins or RNA splice sites; or 5) elimination of antisense open reading frames. Increased expression of nucleic acids in plants can be achieved by utilizing the distribution frequency of codon usage in plants in general or in a particular plant. Methods for optimizing nucleic acid expression in plants can be found in EPA 0359472; EPA 0385962; PCT Application No. WO 91/16432; U.S. Pat. No. 5,380,831; U.S. Pat. No. 5,436,391; Perlack et al., 1991, Proc. Natl. Acad. Sci. USA 88:3324-3328; and Murray et al., 1989, Nucleic Acids Res. 17:477-498.

An isolated polynucleotide of the invention can be optimized such that its distribution frequency of codon usage deviates, preferably, no more than 25% from that of highly expressed plant genes and, more preferably, no more than about 10%. In addition, consideration is given to the percentage G+C content of the degenerate third base (monocotyledons appear to favor G+C in this position, whereas dicotyledons do not). It is also recognized that the XCG (where X is A, T, C, or G) nucleotide is the least preferred codon in dicots, whereas the XTA codon is avoided in both monocots and dicots. Optimized nucleic acids of this invention also preferably have CG and TA doublet avoidance indices closely approximating those of the chosen host plant. More preferably, these indices deviate from that of the host by no more than about 10-15%.

The invention further provides an isolated recombinant expression vector comprising a polynucleotide as described above, wherein expression of the vector in a host cell results in the plant's increased growth and/or yield under normal or water-limited conditions and/or increased tolerance to environmental stress as compared to a wild type variety of the host cell. The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. As used herein with respect to a recombinant expression vector, “operatively linked” is intended to mean that the nucleotide sequence of interest is linked to the regulatory sequence(s) in a manner which allows for expression of the nucleotide sequence (e.g., in a bacterial or plant host cell when the vector is introduced into the host cell). The term “regulatory sequence” is intended to include promoters, enhancers, and other expression control elements (e.g., polyadenylation signals). Such regulatory sequences are well known in the art. Regulatory sequences include those that direct constitutive expression of a nucleotide sequence in many types of host cells and those that direct expression of the nucleotide sequence only in certain host cells or under certain conditions. It will be appreciated by those skilled in the art that the design of the expression vector can depend on such factors as the choice of the host cell to be transformed, the level of expression of polypeptide desired, etc. The expression vectors of the invention can be introduced into host cells to thereby produce polypeptides encoded by nucleic acids as described herein.

Plant gene expression should be operatively linked to an appropriate promoter conferring gene expression in a timely, cell specific, or tissue specific manner. Promoters useful in the expression cassettes of the invention include any promoter that is capable of initiating transcription in a plant cell. Such promoters include, but are not limited to, those that can be obtained from plants, plant viruses, and bacteria that contain genes that are expressed in plants, such as Agrobacterium and Rhizobium.

The promoter may be constitutive, inducible, developmental stage-preferred, cell type-preferred, tissue-preferred, or organ-preferred. Constitutive promoters are active under most conditions. Examples of constitutive promoters include the CaMV 19S and 35S promoters (Odell et al., 1985, Nature 313:810-812), the sX CaMV 35S promoter (Kay et al., 1987, Science 236:1299-1302) the Sep1 promoter, the rice actin promoter (McElroy et al., 1990, Plant Cell 2:163-171), the Arabidopsis actin promoter, the ubiquitan promoter (Christensen et al., 1989, Plant Molec. Biol. 18:675-689), pEmu (Last et al., 1991, Theor. Appl. Genet. 81:581-588), the figwort mosaic virus 35S promoter, the Smas promoter (Velten et al., 1984, EMBO J 3:2723-2730), the super promoter (U.S. Pat. No. 5,955,646), the GRP1-8 promoter, the cinnamyl alcohol dehydrogenase promoter (U.S. Pat. No. 5,683,439), promoters from the T-DNA of Agrobacterium, such as mannopine synthase, nopaline synthase, and octopine synthase, the small subunit of ribulose biphosphate carboxylase (ssuRUBISCO) promoter, and the like.

Inducible promoters are preferentially active under certain environmental conditions, such as the presence or absence of a nutrient or metabolite, heat or cold, light, pathogen attack, anaerobic conditions, and the like. For example, the hsp80 promoter from Brassica is induced by heat shock; the PPDK promoter is induced by light; the PR-1 promoters from tobacco, Arabidopsis, and maize are inducible by infection with a pathogen; and the Adh1 promoter is induced by hypoxia and cold stress. Plant gene expression can also be facilitated via an inducible promoter (For a review, see Gatz, 1997, Annu. Rev. Plant Physiol. Plant Mol. Biol. 48:89-108). Chemically inducible promoters are especially suitable if gene expression is wanted to occur in a time specific manner. Examples of such promoters are a salicylic acid inducible promoter (PCT Application No. WO 95/19443), a tetracycline inducible promoter (Gatz et al., 1992, Plant J. 2: 397-404), and an ethanol inducible promoter (PCT Application No. WO 93/21334).

In one preferred embodiment of the present invention, the inducible promoter is a stress-inducible promoter. For the purposes of the invention, stress-inducible promoters are preferentially active under one or more of the following stresses: sub-optimal conditions associated with salinity, drought, nitrogen, temperature, metal, chemical, pathogenic, and oxidative stresses. Stress inducible promoters include, but are not limited to, Cor78 (Chak et al., 2000, Planta 210:875-883; Hovath et al., 1993, Plant Physiol. 103:1047-1053), Cor15a (Artus et al., 1996, PNAS 93(23):13404-09), Rci2A (Medina et al., 2001, Plant Physiol. 125:1655-66; Nylander et al., 2001, Plant Mol. Biol. 45:341-52; Navarre and Goffeau, 2000, EMBO J. 19:2515-24; Capel et al., 1997, Plant Physiol. 115:569-76), Rd22 (Xiong et al., 2001, Plant Cell 13:2063-83; Abe et al., 1997, Plant Cell 9:1859-68; Iwasaki et al., 1995, Mol. Gen. Genet. 247:391-8), cDet6 (Lang and Palve, 1992, Plant Mol. Biol. 20:951-62), ADH1 (Hoeren et al., 1998, Genetics 149:479-90), KAT1 (Nakamura et al., 1995, Plant Physiol. 109:371-4), KST1 (Müller-Röber et al., 1995, EMBO 14:2409-16), Rha1 (Terryn et al., 1993, Plant Cell 5:1761-9; Terryn et al., 1992, FEBS Lett. 299(3):287-90), ARSK1 (Atkinson et al., 1997, GenBank Accession #L22302, and PCT Application No. WO 97/20057), PtxA (Plesch et al., GenBank Accession #X67427), SbHRGP3 (Ahn et al., 1996, Plant Cell 8:1477-90), GH3 (Liu et al., 1994, Plant Cell 6:645-57), the pathogen inducible PRP1-gene promoter (Ward et al., 1993, Plant. Mol. Biol. 22:361-366), the heat inducible hsp80-promoter from tomato (U.S. Pat. No. 5,187,267), cold inducible alpha-amylase promoter from potato (PCT Application No. WO 96/12814), or the wound-inducible pinll-promoter (European Patent No. 375091). For other examples of drought, cold, and salt-inducible promoters, such as the RD29A promoter, see Yamaguchi-Shinozalei et al., 1993, Mol. Gen. Genet. 236:331-340.

Developmental stage-preferred promoters are preferentially expressed at certain stages of development. Tissue and organ preferred promoters include those that are preferentially expressed in certain tissues or organs, such as leaves, roots, seeds, or xylem. Examples of tissue-preferred and organ-preferred promoters include, but are not limited to fruit-preferred, ovule-preferred, male tissue-preferred, seed-preferred, integument-preferred, tuber-preferred, stalk-preferred, pericarp-preferred, leaf-preferred, stigma-preferred, pollen-preferred, anther-preferred, petal-preferred, sepal-preferred, pedicel-preferred, silique-preferred, stem-preferred, root-preferred promoters, and the like. Seed-preferred promoters are preferentially expressed during seed development and/or germination. For example, seed-preferred promoters can be embryo-preferred, endosperm-preferred, and seed coat-preferred (See Thompson et al., 1989, BioEssays 10:108). Examples of seed-preferred promoters include, but are not limited to, cellulose synthase (celA), Cim1, gamma-zein, globulin-1, maize 19 kD zein (cZ19B1), and the like.

Other suitable tissue-preferred or organ-preferred promoters include the napin-gene promoter from rapeseed (U.S. Pat. No. 5,608,152), the USP-promoter from Vicia faba (Baeumlein et al., 1991, Mol. Gen. Genet. 225(3): 459-67), the oleosin-promoter from Arabidopsis (PCT Application No. WO 98/45461), the phaseolin-promoter from Phaseolus vulgaris (U.S. Pat. No. 5,504,200), the Bce4-promoter from Brassica (PCT Application No. WO 91/13980), or the legumin B4 promoter (LeB4; Baeumlein et al., 1992, Plant Journal, 2(2): 233-9), as well as promoters conferring seed specific expression in monocot plants like maize, barley, wheat, rye, rice, etc. Suitable promoters to note are the Ipt2 or Ipt1-gene promoter from barley (PCT Application No. WO 95/15389 and PCT Application No. WO 95/23230) or those described in PCT Application No. WO 99/16890 (promoters from the barley hordein-gene, rice glutelin gene, rice oryzin gene, rice prolamin gene, wheat gliadin gene, wheat glutelin gene, oat glutelin gene, Sorghum kasirin-gene, and rye secalin gene).

Other promoters useful in the expression cassettes of the invention include, but are not limited to, the major chlorophyll a/b binding protein promoter, histone promoters, the Ap3 promoter, the β-conglycin promoter, the napin promoter, the soybean lectin promoter, the maize 15 kD zein promoter, the 22 kD zein promoter, the 27 kD zein promoter, the g-zein promoter, the waxy, shrunken 1, shrunken 2, and bronze promoters, the Zm13 promoter (U.S. Pat. No. 5,086,169), the maize polygalacturonase promoters (PG) (U.S. Pat. Nos. 5,412,085 and 5,545,546), and the SGB6 promoter (U.S. Pat. No. 5,470,359), as well as synthetic or other natural promoters.

Additional flexibility in controlling heterologous gene expression in plants may be obtained by using DNA binding domains and response elements from heterologous sources (i.e., DNA binding domains from non-plant sources). An example of such a heterologous DNA binding domain is the LexA DNA binding domain (Brent and Ptashne, 1985, Cell 43:729-736).

In a preferred embodiment of the present invention, the polynucleotides listed in Table 1 are expressed in plant cells from higher plants (e.g., the spermatophytes, such as crop plants). A polynucleotide may be “introduced” into a plant cell by any means, including transfection, transformation or transduction, electroporation, particle bombardment, agroinfection, and the like. Suitable methods for transforming or transfecting plant cells are disclosed, for example, using particle bombardment as set forth in U.S. Pat. Nos. 4,945,050; 5,036,006; 5,100,792; 5,302,523; 5,464,765; 5,120,657; 6,084,154; and the like. More preferably, the transgenic corn seed of the invention may be made using Agrobacterium transformation, as described in U.S. Pat. Nos. 5,591,616; 5,731,179; 5,981,840; 5,990,387; 6,162,965; 6,420,630, U.S. patent application publication number 2002/0104132, and the like. Transformation of soybean can be performed using for example a technique described in European Patent No. EP 0424047, U.S. Pat. No. 5,322,783, European Patent No. EP 0397 687, U.S. Pat. No. 5,376,543, or U.S. Pat. No. 5,169,770. A specific example of wheat transformation can be found in PCT Application No. WO 93/07256. Cotton may be transformed using methods disclosed in U.S. Pat. Nos. 5,004,863; 5,159,135; 5,846,797, and the like. Rice may be transformed using methods disclosed in U.S. Pat. Nos. 4,666,844; 5,350,688; 6,153,813; 6,333,449; 6,288,312; 6,365,807; 6,329,571, and the like. Other plant transformation methods are disclosed, for example, in U.S. Pat. Nos. 5,932,782; 6,153,811; 6,140,553; 5,969,213; 6,020,539, and the like. Any plant transformation method suitable for inserting a transgene into a particular plant may be used in accordance with the invention.

According to the present invention, the introduced polynucleotide may be maintained in the plant cell stably if it is incorporated into a non-chromosomal autonomous replicon or integrated into the plant chromosomes. Alternatively, the introduced polynucleotide may be present on an extra-chromosomal non-replicating vector and may be transiently expressed or transiently active.

Another aspect of the invention pertains to an isolated polypeptide having a sequence selected from the group consisting of the polypeptide sequences listed in Table 1. An “isolated” or “purified” polypeptide is free of some of the cellular material when produced by recombinant DNA techniques, or chemical precursors or other chemicals when chemically synthesized. The language “substantially free of cellular material” includes preparations of a polypeptide in which the polypeptide is separated from some of the cellular components of the cells in which it is naturally or recombinantly produced. In one embodiment, the language “substantially free of cellular material” includes preparations of a polypeptide of the invention having less than about 30% (by dry weight) of contaminating polypeptides, more preferably less than about 20% of contaminating polypeptides, still more preferably less than about 10% of contaminating polypeptides, and most preferably less than about 5% contaminating polypeptides.

The determination of activities and kinetic parameters of enzymes is well established in the art. Experiments to determine the activity of any given altered enzyme must be tailored to the specific activity of the wild-type enzyme, which is well within the ability of one skilled in the art. Overviews about enzymes in general, as well as specific details concerning structure, kinetics, principles, methods, applications and examples for the determination of many enzyme activities are abundant and well known to one skilled in the art.

The invention is also embodied in a method of producing a transgenic plant comprising at least one polynucleotide listed in Table 1, wherein expression of the polynucleotide in the plant results in the plant's increased growth and/or yield under normal or water-limited conditions and/or increased tolerance to an environmental stress as compared to a wild type variety of the plant comprising the steps of: (a) introducing into a plant cell an expression vector comprising at least one polynucleotide listed in Table 1, and (b) generating from the plant cell a transgenic plant that expresses the polynucleotide, wherein expression of the polynucleotide in the transgenic plant results in the plant's increased growth and/or yield under normal or water-limited conditions and/or increased tolerance to environmental stress as compared to a wild type variety of the plant. The plant cell may be, but is not limited to, a protoplast, gamete producing cell, and a cell that regenerates into a whole plant. As used herein, the term “transgenic” refers to any plant, plant cell, callus, plant tissue, or plant part, that contains at least one recombinant polynucleotide listed in Table 1. In many cases, the recombinant polynucleotide is stably integrated into a chromosome or stable extra-chromosomal element, so that it is passed on to successive generations.

The present invention also provides a method of increasing a plant's growth and/or yield under normal or water-limited conditions and/or increasing a plant's tolerance to an environmental stress comprising the steps of increasing the expression of at least one polynucleotide listed in Table 1 in the plant. Expression of a protein can be increased by any method known to those of skill in the art.

The effect of the genetic modification on plant growth and/or yield and/or stress tolerance can be assessed by growing the modified plant under less than suitable conditions and then analyzing the growth characteristics and/or metabolism of the plant. Such analysis techniques are well known to one skilled in the art, and include dry weight, wet weight, polypeptide synthesis, carbohydrate synthesis, lipid synthesis, evapotranspiration rates, general plant and/or crop yield, flowering, reproduction, seed setting, root growth, respiration rates, photosynthesis rates, etc., using methods known to those of skill in biotechnology.

The invention is further illustrated by the following examples, which are not to be construed in any way as imposing limitations upon the scope thereof.

### Example 1

### Identification of P. patens Open Reading Frames

cDNA libraries made from plants of the species P. patens (Hedw.) B.S.G. from the collection of the genetic studies section of the University of Hamburg were sequences using standard methods. The plants originated from the strain 16/14 collected by H. L. K. Whitehouse in Gransden Wood, Huntingdonshire (England), which was subcultured from a spore by Engel (1968, Am. J. Bot. 55:438-446).

P. patens partial cDNAs (ESTs) were identified in the P. patens EST sequencing program using the program EST-MAX (Bio-Max (Munich, Germany) The full-length nucleotide cDNA sequences were determined using known methods. The identity and similarity of the amino acid sequences of the disclosed polypeptide sequences to known protein sequences are shown in Tables 2 through 5 (Pairwise Comparison was used with Align and default settings).

TABLE 2
Comparison of EST462 (SEQ ID NO: 2) to
known CBL-interacting protein kinases

| Public Database | | Sequence |
|---|---|---|
| Accession # | Species | Identity (%) |
| ABJ91230 | Populus trichocarpa | 68.50% |
| ABJ91231 | P. trichocarpa | 66.20% |
| NP_001058901 | O. sativa | 65.60% |
| NP_171622 | A. thaliana | 65.40% |
| ABJ91219 | P. trichocarpa | 65.60% |
| EST443 (SEQ ID NO: 77) | P. patens | 58.00% |

TABLE 3
Comparison of EST329 (SEQ ID NO: 4) to known 14-3-3 proteins

| Public Database | | Sequence |
|---|---|---|
| Accession # | Species | Identity (%) |
| BAD12177 | Nicotiana tabacum | 84.20% |
| AAY67798 | Manihot esculenta | 84.10% |
| BAD12176 | Nicotiana tabacum | 83.80% |
| AAC04811 | Fritillaria agrestis | 83.40% |
| Q9SP07 | Lilium longiflorum | 83.40% |
| EST217 | P. patens | 75.5% |

TABLE 4
Comparison of EST373 (SEQ ID NO: 6)
to known RING H2 Zinc finger proteins

| Public Database | | Sequence |
|---|---|---|
| Accession # | Species | Identity (%) |
| AAF27026 | A. thaliana | 20.00% |
| AAD33584 | A. thaliana | 19.50% |
| AAM60957 | A. thaliana | 18.20% |
| NP_198094 | A. thaliana | 18.20% |
| NP_192651 | A. thaliana | 16.80% |

TABLE 5
Comparison of EST548 (SEQ ID NO:
20) to known GTP binding proteins

| Public Database | | Sequence |
|---|---|---|
| Accession # | Species | Identity (%) |
| NP_001055761 | O. sativa | 87.10% |
| BAB84323 | N. tabacum | 86.30% |
| NP_001059259 | O. sativa | 86.30% |
| BAB84324 | N. tabacum | 86.20% |
| ABE82101 | Medicago truncatula | 85.80% |

### Example 2

### Cloning of Full-Length cDNAs from Other Plants

Canola, soybean, rice, maize, linseed, and wheat plants were grown under a variety of conditions and treatments, and different tissues were harvested at various developmental stages. Plant growth and harvesting were done in a strategic manner such that the probability of harvesting all expressable genes in at least one or more of the resulting libraries is maximized. The mRNA was isolated from each of the collected samples, and cDNA libraries were constructed. No amplification steps were used in the library production process in order to minimize redundancy of genes within the sample and to retain expression information. All libraries were 3′ generated from mRNA purified on oligo dT columns. Colonies from the transformation of the cDNA library into E. coli were randomly picked and placed into microtiter plates.

Plasmid DNA was isolated from the E. coli colonies and then spotted on membranes. A battery of 288 ³³P radiolabeled 7-mer oligonucleotides were sequentially hybridized to these membranes. To increase throughput, duplicate membranes were processed. After each hybridization, a blot image was captured during a phosphorimage scan to generate a hybridization profile for each oligonucleotide. This raw data image was automatically transferred to a computer. Absolute identity was maintained by barcoding for the image cassette, filter, and orientation within the cassette. The filters were then treated using relatively mild conditions to strip the bound probes and returned to the hybridization chambers for another round of hybridization. The hybridization and imaging cycle was repeated until the set of 288 oligomers was completed.

After completion of the hybridizations, a profile was generated for each spot (representing a cDNA insert), as to which of the 288 ³³P radiolabeled 7-mer oligonucleotides bound to that particular spot (cDNA insert), and to what degree. This profile is defined as the signature generated from that clone. Each clone's signature was compared with all other signatures generated from the same organism to identify clusters of related signatures. This process “sorts” all of the clones from an organism into clusters before sequencing.

The clones were sorted into various clusters based on their having identical or similar hybridization signatures. A cluster should be indicative of the expression of an individual gene or gene family. A by-product of this analysis is an expression profile for the abundance of each gene in a particular library. One-path sequencing from the 5′ end was used to predict the function of the particular clones by similarity and motif searches in sequence databases.

The full-length DNA sequence of the P. patens RING H2 zinc finger protein (SEQ ID NO:6) was blasted against proprietary databases of canola, soybean, rice, maize, linseed, and wheat cDNAS at an e value of e⁻¹⁰ (Altschul et al., 1997, Nucleic Acids Res. 25: 3389-3402). All the contig hits were analyzed for the putative full length sequences, and the longest clones representing the putative full length contigs were fully sequenced. One homolog from barley, two homologs from Brassica, and three homologs from soybean were identified. The degree of amino acid identity and similarity of these sequences to the closest known public sequences is indicated in Tables 6-11 (Pairwise Comparison was used with Align and default settings).

TABLE 6
Comparison of HV62561245 (SEQ ID NO: 8)
to known RING-H2 zinc finger proteins

| Public Database | | Sequence |
|---|---|---|
| Accession # | Species | Identity (%) |
| NP_001053607 | O. sativa | 62.60% |
| CAH67054 | O. sativa | 62.60% |
| NP_001047725 | O. sativa | 50.20% |
| EAZ31640 | O. sativa | 41.1% |
| ABN08252 | M. truncatula | 36.1% |

TABLE 7
Comparison of BN43173847 (SEQ ID NO: 10)
to known RING-H2 zinc finger proteins

| Public Database | | Sequence |
|---|---|---|
| Accession # | Species | Identity (%) |
| AAM65773 | A. thaliana | 70.50% |
| AAC77829 | A. thaliana | 69.80% |
| NP_188294 | A. thaliana | 68.80% |
| AAW33880 | Populus alba × | 50.50% |
| | Populus tremula | |
| AAM61585 | A. thaliana | 37.40% |

TABLE 8
Comparison of BN46735603 (SEQ ID NO: 12)
to known RING-H2 zinc finger proteins

| Public Database | | Sequence |
|---|---|---|
| Accession # | Species | Identity (%) |
| AAM65773 | A. thaliana | 55.00% |
| AAC77829 | A. thaliana | 54.40% |
| NP_188294 | A. thaliana | 53.70% |
| AAM61585 | A. thaliana | 47.70% |
| NP_567480 | A. thaliana | 47.70% |

TABLE 9
Comparison of GM52504443 (SEQ ID NO: 14)
to known RING-H2 zinc finger proteins

| Public Database | | Sequence |
|---|---|---|
| Accession # | Species | Identity (%) |
| ABE77983 | M. truncatula | 66.10% |
| ABD32383 | M. truncatula | 59.20% |
| AAO45753 | Cucumis melo | 53.80% |
| AAF27026 | A. thaliana | 42.20% |
| AAL86301 | A. thaliana | 41.50% |

TABLE 10
Comparison of GM47122590 (SEQ ID NO: 16)
to known RING-H2 zinc finger proteins

| Public Database | | Sequence |
|---|---|---|
| Accession # | Species | Identity (%) |
| NP_192753 | A. thaliana | 44.90% |
| Q570X5 | A. thaliana | 41.90% |
| NP_192754 | A. thaliana | 40.40% |
| NP_001047138 | O. sativa | 39.5% |
| NP_174614 | A. thaliana | 21.90% |

TABLE 11
Comparison of GM52750153 (SEQ ID NO: 18)
to known RING-H2 zinc finger proteins

| Public Database | | Sequence |
|---|---|---|
| Accession # | Species | Identity (%) |
| NP_001053607 | O. sativa | 33.00% |
| CAH67054 | O. sativa | 33.00% |
| NP_001047725 | O. sativa | 31.60% |
| AAX92760 | O. sativa | 24.50% |
| ABA95805 | O. sativa | 19.40% |

The full-length DNA sequence of the P. patens GTP binding protein (SEQ ID NO:20) was blasted against proprietary databases of canola, soybean, rice, maize, linseed, sunflower, and wheat cDNAS at an e value of e⁻¹⁰ (Altschul et al., 1997, Nucleic Acids Res. 25: 3389-3402). All the contig hits were analyzed for the putative full length sequences, and the longest clones representing the putative full length contigs were fully sequenced. Three homologs from barley, three homologs from Brassica, two homologs from soybean, two homologs from wheat, nine homologs from linseed, three homologs from rice, and six homologs from sunflower were identified. The degree of amino acid identity and similarity of these sequences to the closest known public sequences is indicated in Tables 12-39 (Pairwise Comparison was used with Align and default settings).

TABLE 12
Comparison of GM50181682 (SEQ ID NO:
22) to known GTP binding proteins

| Public Database | | Sequence |
|---|---|---|
| Accession # | Species | Identity (%) |
| NP_190556 | A. thaliana | 92.90% |
| NP_569051 | A. thaliana | 91.30% |
| NP_001049292 | O. sativa | 87.50% |
| BAB08464 | A. thaliana | 82.10% |
| NP_568553 | A. thaliana | 81.00% |

TABLE 13
Comparison of HV62638446 (SEQ ID NO:
24) to known GTP binding proteins

| Public Database | | Sequence |
|---|---|---|
| Accession # | Species | Identity (%) |
| NP_001065511 | O. sativa | 96.90% |
| ABE90431 | M. truncatula | 87.40% |
| BAD07876 | O. sativa | 87.10% |
| AAW67545 | Daucus carota | 86.50% |
| NP_186962 | A. thaliana | 83.90% |

TABLE 14
Comparison of TA56528531 (SEQ ID NO:
26) to known GTP binding proteins

| Public Database | | Sequence |
|---|---|---|
| Accession # | Species | Identity (%) |
| NP_001051716 | O. sativa | 93.00% |
| AAS88430 | O. sativa | 92.10% |
| NP_001059259 | O. sativa | 92.10% |
| CAA04701 | D. carota | 89.80% |
| BAB84323 | N. tabacum | 89.80% |

TABLE 15
Comparison of HV62624858 (SEQ ID NO:
28) to known GTP binding proteins

| Public Database | | Sequence |
|---|---|---|
| Accession # | Species | Identity (%) |
| NP_001061368 | O. sativa | 98.40% |
| ABE83396 | M. truncatula | 92.30% |
| NP_850057 | A. thaliana | 90.70% |
| Q96361 | Brassica rapa | 90.10% |
| XP_416175 | Gallus gallus | 64.30% |

TABLE 16
Comparison of LU61640267 (SEQ ID NO:
30) to known GTP binding proteins

| Public Database | | Sequence |
|---|---|---|
| Accession # | Species | Identity (%) |
| ABB03801 | D. carota | 99.40% |
| AAF65512 | Capsicum annuum | 98.90% |
| AAI22856 | Bos taurus | 98.90% |
| AAR29293 | Medicago sativa | 98.30% |
| ABA40446 | Solanum tuberosum | 98.30% |

TABLE 17
Comparison of LU61872929 (SEQ ID NO:
32) to known GTP binding proteins

| Public Database | | Sequence |
|---|---|---|
| Accession # | Species | Identity (%) |
| O04266 | B. rapa | 95.30% |
| NP_001042942 | O. sativa | 93.30% |
| NP_191815 | A. thaliana | 93.30% |
| ABA81873 | S. tuberosum | 93.30% |
| O04267 | B. rapa | 92.80% |

TABLE 18
Comparison of LU61896092 (SEQ ID NO:
34) to known GTP binding proteins

| Public Database | | Sequence |
|---|---|---|
| Accession # | Species | Identity (%) |
| NP_188935 | A. thaliana | 91.80% |
| NP_001068170 | O. sativa | 85.90% |
| NP_648201 | Drosophila melanogaster | 59.00% |
| XP_623433 | Apis mellifera | 58.50% |
| XP_645417 | Dictyostelium discoideum | 58.10% |

TABLE 19
Comparison of LU61748785 (SEQ ID NO:
36) to known GTP binding proteins

| Public Database | | Sequence |
|---|---|---|
| Accession # | Species | Identity (%) |
| NP_191815 | A. thaliana | 94.30% |
| ABA81873 | S. tuberosum | 94.30% |
| O04266 | B. rapa | 94.30% |
| CAA69699 | Nicotiana plumbaginifolia | 93.80% |
| AAF17254 | N. tabacum | 93.30% |

TABLE 20
Comparison of OS34706416 (SEQ ID NO:
38) to known GTP binding proteins

| Public Database | | Sequence |
|---|---|---|
| Accession # | Species | Identity (%) |
| ABA81873 | S. tuberosum | 94.30% |
| NP_001042942 | O. sativa | 93.30% |
| AAC32610 | Avena fatua | 92.70% |
| BAA13463 | N. tabacum | 92.70% |
| CAA69699 | N. plumbaginifolia | 92.20% |

TABLE 21
Comparison of GM49750953 (SEQ ID NO:
40) to known GTP binding proteins

| Public Database | | Sequence |
|---|---|---|
| Accession # | Species | Identity (%) |
| ABA81873 | S. tuberosum | 94.30% |
| NP_001042942 | O. sativa | 93.30% |
| AAC32610 | A. fatua | 92.70% |
| BAA13463 | N. abacum | 92.70% |
| CAA69699 | N. plumbaginifolia | 92.20% |

TABLE 22
Comparison of HA66696606 (SEQ ID NO:
42) to known GTP binding proteins

| Public Database | | Sequence |
|---|---|---|
| Accession # | Species | Identity (%) |
| ABB03801 | D. carota | 99.40% |
| AAR29293 | M. sativa | 99.40% |
| ABA40446 | S. tuberosum | 99.40% |
| NP_001044599 | O. sativa | 98.90% |
| AAF65512 | C. annuum | 98.90% |

TABLE 23
Comparison of HA66783477 (SEQ ID NO:
44) to known GTP binding proteins

| Public Database | | Sequence |
|---|---|---|
| Accession # | Species | Identity (%) |
| ABA81873 | S. tuberosum | 96.40% |
| CAA69699 | N. plumbaginifolia | 95.30% |
| BAA13463 | N. tabacum | 94.80% |
| ABA46770 | S. tuberosum | 93.30% |
| NP_001042942 | O. sativa | 92.70% |

TABLE 24
Comparison of HA66705690 (SEQ ID NO:
46) to known GTP binding proteins

| Public Database | | Sequence |
|---|---|---|
| Accession # | Species | Identity (%) |
| CAA98161 | L. japonicus | 91.10% |
| CAA98162 | L. japonicus | 90.60% |
| BAA02117 | P. sativum | 90.10% |
| BAA02118 | P. sativum | 90.10% |
| AAB97115 | G. max | 89.20% |

TABLE 25
Comparison of TA59921546 (SEQ ID NO:
48) to known GTP binding proteins

| Public Database | | Sequence |
|---|---|---|
| Accession # | Species | Identity (%) |
| NP_001061368 | O. sativa | 97.30% |
| ABE83396 | M. truncatula | 92.30% |
| NP_850057 | A. thaliana | 89.60% |
| Q96361 | B. rapa | 89.00% |
| XP_636876 | D. discoideum | 64.50% |

TABLE 26
Comparison of HV62657638 (SEQ ID NO:
50) to known GTP binding proteins

| Public Database | | Sequence |
|---|---|---|
| Accession # | Species | Identity (%) |
| NP_001055761 | O. sativa | 95.80% |
| NP_001059259 | O. sativa | 94.00% |
| NP_001051716 | O. sativa | 93.50% |
| ABE82101 | M. truncatula | 92.10% |
| AAS88430 | O. sativa | 91.60% |

TABLE 27
Comparison of BN43540204 (SEQ ID NO:
52) to known GTP binding proteins

| Public Database | | Sequence |
|---|---|---|
| Accession # | Species | Identity (%) |
| AAB04618 | B. rapa | 99.00% |
| NP_187779 | A. thaliana | 98.10% |
| AAD10389 | Petunia axillaris × | 85.90% |
| | Petunia integrifolia | |
| AAA80679 | Solanum lycopersicum | 85.90% |
| CAA66447 | Lotus japonicus | 84.00% |

TABLE 28
Comparison of BN45139744 (SEQ ID NO:
54) to known GTP binding proteins

| Public Database | | Sequence |
|---|---|---|
| Accession # | Species | Identity (%) |
| NP_171715 | A. thaliana | 96.60% |
| AAB97115 | G. max | 93.10% |
| BAA00832 | A. thaliana | 92.60% |
| BAA02118 | Pisum sativum | 92.20% |
| CAA98161 | L. japonicus | 90.20% |

TABLE 29
Comparison of BN43613585 (SEQ ID NO:
56) to known GTP binding proteins

| Public Database | | Sequence |
|---|---|---|
| Accession # | Species | Identity (%) |
| NP_200792 | A. thaliana | 56.40% |
| CAA98173 | L. japonicus | 56.00% |
| ABE82101 | M. truncatula | 52.80% |
| BAB84326 | N. tabacum | 52.30% |
| BAB84324 | N. tabacum | 52.30% |

TABLE 30
Comparison of LU61965240 (SEQ ID NO:
58) to known GTP binding proteins

| Public Database | | Sequence |
|---|---|---|
| Accession # | Species | Identity (%) |
| CAA98160 | L. japonicus | 92.60% |
| BAA02116 | P. sativum | 92.10% |
| BAA76422 | Cicer arietinum | 90.60% |
| NP_193486 | A. thaliana | 90.60% |
| ABD65068 | Brassica oleracea | 90.60% |

TABLE 31
Comparison of LU62294414 (SEQ ID NO:
60) to known GTP binding proteins

| Public Database | | Sequence |
|---|---|---|
| Accession # | Species | Identity (%) |
| NP_568121 | A. thaliana | 81.10% |
| CAA98163 | L. japonicus | 79.70% |
| NP_187602 | A. thaliana | 73.60% |
| NP_001048954 | O. sativa | 71.20% |
| NP_001064756 | O. sativa | 68.50% |

TABLE 32
Comparison of LU61723544 (SEQ ID NO:
62) to known GTP binding proteins

| Public Database | | Sequence |
|---|---|---|
| Accession # | Species | Identity (%) |
| ABE82101 | M. truncatula | 97.70% |
| BAB84324 | N. tabacum | 94.90% |
| CAA90080 | P. sativum | 94.40% |
| BAB84326 | N. tabacum | 94.40% |
| BAB84323 | N. tabacum | 94.40% |

TABLE 33
Comparison of LU61871078 (SEQ ID NO:
64) to known GTP binding proteins

| Public Database | | Sequence |
|---|---|---|
| Accession # | Species | Identity (%) |
| CAA66447 | L. japonicus | 91.50% |
| AAD10389 | P. axillaris × | 90.60% |
| | P. integrifolia | |
| BAA02115 | P. sativum | 90.50% |
| AAA80679 | S. lycopersicum | 90.10% |
| AAA34003 | G. max | 89.60% |

TABLE 34
Comparison of LU61569070 (SEQ ID NO:
66) to known GTP binding proteins

| Public Database | | Sequence |
|---|---|---|
| Accession # | Species | Identity (%) |
| CAA98160 | L. japonicus | 93.60% |
| BAA02116 | P. sativum | 93.10% |
| BAA76422 | C. arietinum | 91.60% |
| NP_001042202 | O. sativa | 91.10% |
| CAC39050 | O. sativa | 91.10% |

TABLE 35
Comparison of OS34999273 (SEQ ID NO:
68) to known GTP binding proteins

| Public Database | | Sequence |
|---|---|---|
| Accession # | Species | Identity (%) |
| BAA02117 | P. sativum | 97.00% |
| CAA98161 | L. japonicus | 95.60% |
| CAA98162 | L. japonicus | 95.10% |
| AAB97115 | G. max | 92.10% |
| BAA02118 | P. sativum | 91.10% |

TABLE 36
Comparison of HA66779896 (SEQ ID NO:
70) to known GTP binding proteins

| Public Database | | Sequence |
|---|---|---|
| Accession # | Species | Identity (%) |
| CAA98160 | L. japonicus | 93.10% |
| CAA69701 | N. plumbaginifolia | 92.10% |
| AAA80678 | S. lycopersicum | 92.10% |
| BAA76422 | C. arietinum | 91.60% |
| ABD65068 | B. oleracea | 91.10% |

TABLE 37
Comparison of OS32667913 (SEQ ID NO:
72) to known GTP binding proteins

| Public Database | | Sequence |
|---|---|---|
| Accession # | Species | Identity (%) |
| ABD59352 | Saccharum officinarum | 90.00% |
| ABD59353 | S. officinarum | 89.50% |
| P16976 | Zea mays | 86.10% |
| 1707300A | Z. mays | 85.20% |
| CAA66447 | L. japonicus | 78.50% |

TABLE 38
Comparison of HA66453181 (SEQ ID NO:
74) to known GTP binding proteins

| Public Database | | Sequence |
|---|---|---|
| Accession # | Species | Identity (%) |
| ABK96799 | S. tuberosum | 89.20% |
| CAA51011 | N. tabacum | 89.20% |
| BAA76422 | C. arietinum | 89.20% |
| CAA98160 | L. japonicus | 89.20% |
| CAA69701 | N. plumbaginifolia | 88.70% |

TABLE 39
Comparison of HA66709897 (SEQ ID NO:
76) to known GTP binding proteins

| Public Database | | Sequence |
|---|---|---|
| Accession # | Species | Identity (%) |
| AAD10389 | P. axillaris × | 94.10% |
| | P. integrifolia | |
| AAA80679 | S. lycopersicum | 93.10% |
| CAA66447 | L. japonicus | 93.00% |
| BAA02115 | P. sativum | 89.60% |
| AAA34003 | G. max | 89.60% |

### Example 3

### Stress-Tolerant Arabidopsis Plants

A fragment containing the P. patens polynucleotide was ligated into a binary vector containing a selectable marker gene. The resulting recombinant vector contained the corresponding gene in the sense orientation under the constitutive super promoter. The recombinant vectors were transformed into Agrobacterium tumefaciens C58C1 and PMP90 plants according to standard conditions. A. thaliana ecotype C24 plants were grown and transformed according to standard conditions. T1 plants were screened for resistance to the selection agent conferred by the selectable marker gene, and T1 seeds were collected.

The P. patens polynucleotides were overexpressed in A. thaliana under the control of a constitutive promoter. T2 and/or T3 seeds were screened for resistance to the selection agent conferred by the selectable marker gene on plates, and positive plants were transplanted into soil and grown in a growth chamber for 3 weeks. Soil moisture was maintained throughout this time at approximately 50% of the maximum water-holding capacity of soil.

The total water lost (transpiration) by the plant during this time was measured. After 3 weeks, the entire above-ground plant material was collected, dried at 65° C. for 2 days and weighed. The ratio of above-ground plant dry weight (DW) to plant water use is water use efficiency (WUE). Tables 40 through 43 present WUE and DW for independent transformation events (lines) of transgenic plants overexpressing the P. patens polynucleotides. Least square means (LSM), standard errors, and significant value (P) of a line compared to wild-type controls from an Analysis of Variance are presented. The percent improvement of each transgenic line as compared to wild-type control plants for WUE and DW is also presented.

TABLE 40
A. thaliana lines overexpressing EST462 (SEQ ID NO: 2).

| Meas- | | | | Standard | % | |
|---|---|---|---|---|---|---|
| urement | Genotype | Line | LSM | Error | Improvement | P |
| DW | Wild-type | | 0.108 | 0.006 | | |
| | | 1 | 0.147 | 0.016 | 36 | 0.027 |
| | | 2 | 0.152 | 0.018 | 41 | 0.0208 |
| | | 3 | 0.168 | 0.018 | 56 | 0.0017 |
| | | 8 | 0.177 | 0.018 | 64 | 0.0004 |
| | | 5 | 0.178 | 0.018 | 64 | 0.0003 |
| | | 10 | 0.230 | 0.016 | 112 | <.0001 |
| WUE | Wild-type | | 1.951 | 0.069 | | |
| | | 8 | 2.156 | 0.195 | 10 | 0.3249 |
| | | 3 | 2.266 | 0.195 | 16 | 0.1308 |
| | | 5 | 2.308 | 0.195 | 18 | 0.0871 |
| | | 10 | 2.475 | 0.178 | 27 | 0.0069 |

TABLE 41
A. thaliana lines overexpressing EST329 (SEQ ID NO: 4)

| Meas- | | | | Standard | % | |
|---|---|---|---|---|---|---|
| urement | Genotype | Line | LSM | Error | Improvement | P |
| DW | Wild type | | 0.178 | 0.007 | | |
| | | 1 | 0.224 | 0.021 | 26 | 0.0414 |
| | | 9 | 0.229 | 0.021 | 29 | 0.0251 |
| | | 8 | 0.230 | 0.021 | 30 | 0.0205 |
| | | 10 | 0.236 | 0.021 | 33 | 0.01 |
| | | 7 | 0.241 | 0.021 | 35 | 0.0055 |
| | | 3 | 0.266 | 0.021 | 49 | 0.0001 |
| | | 4 | 0.284 | 0.021 | 59 | <.0001 |
| | | 5 | 0.290 | 0.021 | 63 | <.0001 |
| | | 2 | 0.311 | 0.021 | 75 | <.0001 |
| WUE | Wild type | | 1.895 | 0.051 | | |
| | | 4 | 1.997 | 0.158 | 5 | 0.5381 |
| | | 2 | 2.069 | 0.158 | 9 | 0.2972 |
| | | 10 | 2.077 | 0.158 | 10 | 0.2757 |
| | | 9 | 2.105 | 0.158 | 11 | 0.2071 |
| | | 8 | 2.238 | 0.158 | 18 | 0.0403 |
| | | 5 | 2.378 | 0.158 | 26 | 0.0041 |
| | | 7 | 2.446 | 0.158 | 29 | 0.0011 |

TABLE 42
A. thaliana lines overexpressing EST373 (SEQ ID NO: 6)

| Meas- | | | | Standard | % | |
|---|---|---|---|---|---|---|
| urement | Genotype | Line | LSM | Error | Improvement | P |
| DW | Wild type | | 0.099 | 0.017 | | |
| | | 7 | 0.131 | 0.020 | 32 | 0.2358 |
| WUE | Wild type | | 1.543 | 0.106 | | |
| | | 7 | 1.937 | 0.156 | 26 | 0.0479 |

TABLE 43
A. thaliana lines overexpressing EST548 (SEQ ID NO: 20).

| Meas- | | | | Standard | % | |
|---|---|---|---|---|---|---|
| urement | Genotype | Line | LSM | Error | Improvement | P |
| DW | Wild-type | | 0.114 | 0.00582 | — | — |
| | | 2 | 0.158 | 0.020 | 39 | 0.0367 |
| | | 1 | 0.164 | 0.018 | 43 | 0.0098 |
| | | 10 | 0.167 | 0.015 | 46 | 0.0014 |
| | | 7 | 0.169 | 0.018 | 49 | 0.004 |
| | | 8 | 0.170 | 0.015 | 49 | 0.0008 |
| | | 4 | 0.186 | 0.018 | 63 | 0.0002 |
| WUE | Wild-type | | 1.958 | 0.055 | — | — |
| | | 2 | 2.117 | 0.191 | 8 | 0.4253 |
| | | 10 | 2.210 | 0.145 | 13 | 0.1051 |
| | | 7 | 2.302 | 0.171 | 18 | 0.0574 |
| | | 8 | 2.325 | 0.145 | 19 | 0.0189 |
| | | 1 | 2.481 | 0.171 | 27 | 0.0041 |
| | | 4 | 2.518 | 0.171 | 29 | 0.0022 |

### Example 4

### Stress-Tolerant Rapeseed/Canola Plants

Canola cotyledonary petioles of 4 day-old young seedlings are used as explants for tissue culture and transformed according to EP1566443. The commercial cultivar Westar (Agriculture Canada) is the standard variety used for transformation, but other varieties can be used. A. tumefaciens GV3101:pMP90RK containing a binary vector is used for canola transformation. The standard binary vector used for transformation is pSUN (WO02/00900), but many different binary vector systems have been described for plant transformation (e.g. An, G. in Agrobacterium Protocols, Methods in Molecular Biology vol 44, pp 47-62, Gartland K M A and M R Davey eds. Humana Press, Totowa, N.J.). A plant gene expression cassette comprising a selection marker gene and a plant promoter regulating the transcription of the cDNA encoding the polynucleotide is employed. Various selection marker genes can be used including the mutated acetohydroxy acid synthase (AHAS) gene disclosed in U.S. Pat. Nos. 5,767,366 and 6,225,105. A suitable promoter is used to regulate the trait gene to provide constitutive, developmental, tissue or environmental regulation of gene transcription.

Canola seeds are surface-sterilized in 70% ethanol for 2 min, incubated for 15 min in 55° C. warm tap water and then in 1.5% sodium hypochlorite for 10 minutes, followed by three rinses with sterilized distilled water. Seeds are then placed on MS medium without hormones, containing Gamborg B5 vitamins, 3% sucrose, and 0.8% Oxoidagar. Seeds are germinated at 24° C. for 4 days in low light (<50 μMol/m²s, 16 hours light). The cotyledon petiole explants with the cotyledon attached are excised from the in vitro seedlings, and inoculated with Agrobacterium by dipping the cut end of the petiole explant into the bacterial suspension. The explants are then cultured for 3 days on MS medium including vitamins containing 3.75 mg/l BAP, 3% sucrose, 0.5 g/l MES, pH 5.2, 0.5 mg/l GA3, 0.8% Oxoidagar at 24° C., 16 hours of light. After three days of co-cultivation with Agrobacterium, the petiole explants are transferred to regeneration medium containing 3.75 mg/l BAP, 0.5 mg/l GA3, 0.5 g/l MES, pH 5.2, 300 mg/l timentin and selection agent until shoot regeneration. As soon as explants start to develop shoots, they are transferred to shoot elongation medium (A6, containing full strength MS medium including vitamins, 2% sucrose, 0.5% Oxoidagar, 100 mg/l myo-inositol, 40 mg/l adenine sulfate, 0.5 g/l MES, pH 5.8, 0.0025 mg/l BAP, 0.1 mg/l IBA, 300 mg/l timentin and selection agent).

Samples from both in vitro and greenhouse material of the primary transgenic plants (T0) are analyzed by qPCR using TaqMan probes to confirm the presence of T-DNA and to determine the number of T-DNA integrations.

Seed is produced from the primary transgenic plants by self-pollination. The second-generation plants are grown in greenhouse conditions and self-pollinated. The plants are analyzed by qPCR using TaqMan probes to confirm the presence of T-DNA and to determine the number of T-DNA integrations. Homozygous transgenic, heterozygous transgenic and azygous (null transgenic) plants are compared for their stress tolerance, for example, in the assays described in Example 3, and for yield, both in the greenhouse and in field studies.

### Example 5

### Screening for Stress-Tolerant Rice Plants

Transgenic rice plants comprising a polynucleotide of the invention are generated using known methods. Approximately 15 to 20 independent transformants (T0) are generated. The primary transformants are transferred from tissue culture chambers to a greenhouse for growing and harvest of T1 seeds. Five events of the T1 progeny segregated 3:1 for presence/absence of the transgene are retained. For each of these events, 10 T1 seedlings containing the transgene (hetero- and homozygotes), and 10 T1 seedlings lacking the transgene (nullizygotes) are selected by visual marker screening. The selected T1 plants are transferred to a greenhouse. Each plant receives a unique barcode label to link unambiguously the phenotyping data to the corresponding plant. The selected T1 plants are grown on soil in 10 cm diameter pots under the following environmental settings: photoperiod=11.5 h, daylight intensity=30,000 lux or more, daytime temperature=28° C. or higher, night time temperature=22° C., relative humidity=60-70%. Transgenic plants and the corresponding nullizygotes are grown side-by-side at random positions. From the stage of sowing until the stage of maturity, the plants are passed several times through a digital imaging cabinet. At each time point digital, images (2048×1536 pixels, 16 million colours) of each plant are taken from at least 6 different angles.

The data obtained in the first experiment with T1 plants are confirmed in a second experiment with T2 plants. Lines that have the correct expression pattern are selected for further analysis. Seed batches from the positive plants (both hetero- and homozygotes) in T1 are screened by monitoring marker expression. For each chosen event, the heterozygote seed batches are then retained for T2 evaluation. Within each seed batch, an equal number of positive and negative plants are grown in the greenhouse for evaluation.

Transgenic plants are screened for their improved growth and/or yield and/or stress tolerance, for example, using the assays described in Example 3, and for yield, both in the greenhouse and in field studies.

### Example 6

### Stress-Tolerant Soybean Plants

The polynucleotides of Tables 1 and 2 are transformed into soybean using the methods described in commonly owned copending international application number WO 2005/121345, the contents of which are incorporated herein by reference.

The transgenic plants generated are then screened for their improved growth under water-limited conditions and/or drought, salt, and/or cold tolerance, for example, using the assays described in Example 3, and for yield, both in the greenhouse and in field studies.

### Example 7

### Stress-Tolerant Wheat Plants

Transformation of wheat is performed with the method described by Ishida et al., 1996, Nature Biotech. 14745-50. Immature embryos are co-cultivated with Agrobacterium tumefaciens that carry “super binary” vectors, and transgenic plants are recovered through organogenesis. This procedure provides a transformation efficiency between 2.5% and 20%. The transgenic plants are then screened for their improved growth and/or yield under water-limited conditions and/or stress tolerance, for example, is the assays described in Example 3, and for yield, both in the greenhouse and in field studies.

### Example 8

### Stress-Tolerant Corn Plants

Agrobacterium cells harboring the genes and the maize ahas gene on the same plasmid are grown in YP medium supplemented with appropriate antibiotics for 1-3 days. A loop of Agrobacterium cells is collected and suspended in 1.5 ml M-LS-002 medium (LS-inf) and the tube containing Agrobacterium cells is kept on a shaker for 1-4 hours at 1,000 rpm.

Corncobs [genotype J553x(HIIIAxA188)] are harvested at 7-12 days after pollination. The cobs are sterilized in 20% Clorox solution for 15 minutes followed by thorough rinse with sterile water. Immature embryos with size 0.8-2.0 mm are dissected into the tube containing Agrobacterium cells in LS-inf solution.

Agro-infection is carried out by keeping the tube horizontally in the laminar hood at room temperature for 30 minutes. Mixture of the agro infection is poured on to a plate containing the co-cultivation medium (M-LS-011). After the liquid agro-solution is piped out, the embryos transferred to the surface of a filter paper that is placed on the agar co-cultivation medium. The excess bacterial solution is removed with a pipette. The embryos are placed on the co-cultivation medium with scutellum side up and cultured in the dark at 22° C. for 2-4 days.

Embryos are transferred to M-MS-101 medium without selection. Seven to ten days later, embryos are transferred to M-LS-401 medium containing 0.50 μM imazethapyr and grown for 4 weeks (two 2-week transfers) to select for transformed callus cells. Plant regeneration is initiated by transferring resistant calli to M-LS-504 medium supplemented with 0.75 μM imazethapyr and grown under light at 25-27° C. for two to three weeks. Regenerated shoots are then transferred to rooting box with M-MS-618 medium (0.5 μM imazethapyr). Plantlets with roots are transferred to potting mixture in small pots in the greenhouse and after acclimatization are then transplanted to larger pots and maintained in greenhouse till maturity.

The copy number of the transgene in each plantlet is assayed using Taqman analysis of genomic DNA, and transgene expression is assayed using qRT-PCR of total RNA isolated from leaf samples.

Using assays such as the assay described in Example 3, each of these plants is uniquely labeled, sampled and analyzed for transgene copy number. Transgene positive and negative plants are marked and paired with similar sizes for transplanting together to large pots. This provides a uniform and competitive environment for the transgene positive and negative plants. The large pots are watered to a certain percentage of the field water capacity of the soil depending the severity of water-stress desired. The soil water level is maintained by watering every other day. Plant growth and physiology traits such as height, stem diameter, leaf rolling, plant wilting, leaf extension rate, leaf water status, chlorophyll content and photosynthesis rate are measured during the growth period. After a period of growth, the above ground portion of the plants is harvested, and the fresh weight and dry weight of each plant are taken. A comparison of the drought tolerance phenotype between the transgene positive and negative plants is then made.

Using assays such as the assay described in Example 3, the pots are covered with caps that permit the seedlings to grow through but minimize water loss. Each pot is weighed periodically and water added to maintain the initial water content. At the end of the experiment, the fresh and dry weight of each plant is measured, the water consumed by each plant is calculated and WUE of each plant is computed. Plant growth and physiology traits such as WUE, height, stem diameter, leaf rolling, plant wilting, leaf extension rate, leaf water status, chlorophyll content and photosynthesis rate are measured during the experiment. A comparison of WUE phenotype between the transgene positive and negative plants is then made.

Using assays such as the assay described in Example 3, these pots are kept in an area in the greenhouse that has uniform environmental conditions, and cultivated optimally. Each of these plants is uniquely labeled, sampled and analyzed for transgene copy number. The plants are allowed to grow under theses conditions until they reach a predefined growth stage. Water is then withheld. Plant growth and physiology traits such as height, stem diameter, leaf rolling, plant wilting, leaf extension rate, leaf water status, chlorophyll content and photosynthesis rate are measured as stress intensity increases. A comparison of the dessication tolerance phenotype between transgene positive and negative plants is then made.

Segregating transgenic corn seeds for a transformation event are planted in small pots for testing in a cycling drought assay. These pots are kept in an area in the greenhouse that has uniform environmental conditions, and cultivated optimally. Each of these plants is uniquely labeled, sampled and analyzed for transgene copy number. The plants are allowed to grow under theses conditions until they reach a predefined growth stage. Plants are then repeatedly watered to saturation at a fixed interval of time. This water/drought cycle is repeated for the duration of the experiment. Plant growth and physiology traits such as height, stem diameter, leaf rolling, leaf extension rate, leaf water status, chlorophyll content and photosynthesis rate are measured during the growth period. At the end of the experiment, the plants are harvested for above-ground fresh and dry weight. A comparison of the cycling drought tolerance phenotype between transgene positive and negative plants is then made.

In order to test segregating transgenic corn for drought tolerance under rain-free conditions, managed-drought stress at a single location or multiple locations is used. Crop water availability is controlled by drip tape or overhead irrigation at a location which has less than 10 cm rainfall and minimum temperatures greater than 5° C. expected during an average 5 month season, or a location with expected in-season precipitation intercepted by an automated “rain-out shelter” which retracts to provide open field conditions when not required. Standard agronomic practices in the area are followed for soil preparation, planting, fertilization and pest control. Each plot is sown with seed segregating for the presence of a single transgenic insertion event. A Taqman transgene copy number assay is used on leaf samples to differentiate the transgenics from null-segregant control plants. Plants that have been genotyped in this manner are also scored for a range of phenotypes related to drought-tolerance, growth and yield. These phenotypes include plant height, grain weight per plant, grain number per plant, ear number per plant, above ground dry-weight, leaf conductance to water vapor, leaf CO₂ uptake, leaf chlorophyll content, photosynthesis-related chlorophyll fluorescence parameters, water use efficiency, leaf water potential, leaf relative water content, stem sap flow rate, stem hydraulic conductivity, leaf temperature, leaf reflectance, leaf light absorptance, leaf area, days to flowering, anthesis-silking interval, duration of grain fill, osmotic potential, osmotic adjustment, root size, leaf extension rate, leaf angle, leaf rolling and survival. All measurements are made with commercially available instrumentation for field physiology, using the standard protocols provided by the manufacturers. Individual plants are used as the replicate unit per event.

In order to test non-segregating transgenic corn for drought tolerance under rain-free conditions, managed-drought stress at a single location or multiple locations is used. Crop water availability is controlled by drip tape or overhead irrigation at a location which has less than 10 cm rainfall and minimum temperatures greater than 5° C. expected during an average 5 month season, or a location with expected in-season precipitation intercepted by an automated “rain-out shelter” which retracts to provide open field conditions when not required. Standard agronomic practices in the area are followed for soil preparation, planting, fertilization and pest control. Trial layout is designed to pair a plot containing a non-segregating transgenic event with an adjacent plot of null-segregant controls. A null segregant is progeny (or lines derived from the progeny) of a transgenic plant that does not contain the transgene due to Mendelian segregation. Additional replicated paired plots for a particular event are distributed around the trial. A range of phenotypes related to drought-tolerance, growth and yield are scored in the paired plots and estimated at the plot level. When the measurement technique could only be applied to individual plants, these are selected at random each time from within the plot. These phenotypes include plant height, grain weight per plant, grain number per plant, ear number per plant, above ground dry-weight, leaf conductance to water vapor, leaf CO₂ uptake, leaf chlorophyll content, photosynthesis-related chlorophyll fluorescence parameters, water use efficiency, leaf water potential, leaf relative water content, stem sap flow rate, stem hydraulic conductivity, leaf temperature, leaf reflectance, leaf light absorptance, leaf area, days to flowering, anthesis-silking interval, duration of grain fill, osmotic potential, osmotic adjustment, root size, leaf extension rate, leaf angle, leaf rolling and survival. All measurements are made with commercially available instrumentation for field physiology, using the standard protocols provided by the manufacturers. Individual plots are used as the replicate unit per event.

To perform multi-location testing of transgenic corn for drought tolerance and yield, five to twenty locations encompassing major corn growing regions are selected. These are widely distributed to provide a range of expected crop water availabilities based on average temperature, humidity, precipitation and soil type. Crop water availability is not modified beyond standard agronomic practices. Trial layout is designed to pair a plot containing a non-segregating transgenic event with an adjacent plot of null-segregant controls. A range of phenotypes related to drought-tolerance, growth and yield are scored in the paired plots and estimated at the plot level. When the measurement technique could only be applied to individual plants, these are selected at random each time from within the plot. These phenotypes included plant height, grain weight per plant, grain number per plant, ear number per plant, above ground dry-weight, leaf conductance to water vapor, leaf CO₂ uptake, leaf chlorophyll content, photosynthesis-related chlorophyll fluorescence parameters, water use efficiency, leaf water potential, leaf relative water content, stem sap flow rate, stem hydraulic conductivity, leaf temperature, leaf reflectance, leaf light absorptance, leaf area, days to flowering, anthesis-silking interval, duration of grain fill, osmotic potential, osmotic adjustment, root size, leaf extension rate, leaf angle, leaf rolling and survival. All measurements are made with commercially available instrumentation for field physiology, using the standard protocols provided by the manufacturers. Individual plots are used as the replicate unit per event.

APPENDIX
cDNA sequence of EST462 from P. patens (SEQ ID NO: 1):
atcccgggtgtaaggtggaggaatggcactgtgacacacggctgatttttgaagaaacgagctccgggtgaaaaatgaaaat
gagttgcggtgcaggatgtggaagcgttcgtcagacagcatgagaagatttgtgtgcccagactctttttattgtatgttagggaag
gaaagatatcgcgaaaccagcgcaagactgagaagggtgaaagttagataggttacttacgtacaagcaaacatgactacc
gcgacaccaagtatcccggctacgaacgtggagcgcacgcgggtcggcaaatatgatctcggcaagaccctgggagagg
gcacatttgccaaagtcaaggtggctaagcacatcgacactggccatactgttgccataaagattttggacaaggacaagattc
tcaagcataagatggttgagcagatcaaaagagaaatatctaccatgaagctagtgaagcacccttacgtcgtccagctgttg
gaagttatggccagcaggacaaaaatctatattgtgctggagtatgttacaggtggcgaacttttcaacaagattgctcaacaag
gaaggctgtcagaggacgacgcaaggaaatactttcagcagctcattgatgcagttgattattgccacagccggcaagtttttca
tagagatttgaagccagagaatctccttctggatgcgaaggggagcttgaaaatttcggactttggtttgagtgcgctaccgcag
caatttagggctgatggattattacacacaacttgcggaacacccaattatgtggctcctgaggtgattatggataagggatattc
gggcgctactgctgatttgtggtcttgcggtgtcatcttatacgtgctgatggctgggtacttgccttttgaggagcccactattatggc
tttgtacaagaagatatatcgggctcaattctcatggcctccctggttcccgtcaggtgcccggaaattaatttcaaagatattggat
cccaaccctagaactcgcatctcagcagctgaaatttataaaaatgattggttcaagaagggatacactccagctcagtttgacc
gagaagctgatgtcaaccttgatgatgtgaatgctatcttcagcggttcacaagaacatatagttgtagaaaggaaggaatcaa
aaccggttactatgaacgcttttgagctcatctctttgtcttcgggcctcaatctttctagtttgtttgagacaaaagagattcctgaaa
aggaggacacgcggtttacaagcaagaaatctgccaaagagatcatcagttcaatcgaggaagctgcgaagcccttgggct
ttaatgttcagaagcgagattataagatgaagttacaaggagacaagctgggcaggaagggacatctttcagtctcaaccga
ggtgttcgaggtggcgccttctctttacatggttgagttacagaagaacagtggtgatacattggagtataaccatttttacaagaat
ctttccaagggcctaaaagacatagtgtggaaagcagaccctcttcctgcatgtgaacaaaagtagacgcttccgctacggctt
caaaataagcccgtgccgtgaagtacccacatctcctcacttggcatctcagttaacgc
The EST 462 cDNA is translated into the following amino acid sequence (SEQ ID NO: 2):
mttatpsipatnvertrvgkydlgktlgegtfakvkvakhidtghtvaikildkdkilkhkmveqikreistmklvkhpyvvqllevm
asrtkiyivleyvtggelfnkiaqqgrlseddarkyfqqlidavdychsrqvfhrdlkpenllldakgslkisdfglsalpqqfradgllh
ttcgtpnyvapevimdkgysgatadlwscgvilyvlmagylpfeeptimalykkiyraqfswppwfpsgarkliskildpnprtris
aaeiykndwfkkgytpaqfdreadvnlddvnaifsgsqehivverkeskpvtmnafelislssglnlsslfetkeipekedtrftsk
ksakeiissieeaakplgfnvqkrdykmklqgdklgrkghlsvstevfevapslymvelqknsgdtleynhfyknlskglkdivw
kadplpaceqk
cDNA sequence of EST329 from P. patens (SEQ ID NO: 3):
atcccgggctcgctcgcttgggtgcagtaacgaccgagatcgaccatggcgacggaggcgcgcgaggagaatgtgtacatg
gccaagctggccgagcaggccgagcgctacgacgagatggtggaggccatggagaaggtggccaagaccgtcgacacc
gaggagctcaccgtcgaagagcgcaacttgttgtctgtggcttacaagaacgtgattggcgctcggagggcgtcgtggaggat
catctcctccatcgagcagaaggaggagagcaagggaaacgacgagcacgtttccgccatcaaggagtaccgtggcaag
gtggagtctgagttgagcaccatctgtgacagtattcttaagcttctggatacccacctgatccctacttctagctctggggagtcga
aagttttctacttgaagatgaagggtgattatcacaggtacttggctgagtttaagaccggggccgagaggaaggaagctgctg
aagcgacattgttggcgtataagtctgctcaagatattgcgttgacagagttggctcctacccaccccatcagactgggtttggca
ttgaacttctctgtgttttattacgagattcttaactcaccagatcgggcgtgcactcttgcgaagcaggcatttgatgaagcgatcg
ctgagcttgatactcttggagaggagtcttacaaggatagcactcttattatgcagctcctccgcgacaacctgacgttgtggacct
ctgatatgcaggatgaggtcggccccgaggtcaaggatgccaaagttgatgatgctgagcactgaagtggaacttaagctata
tttatctttgcacagcagagaggtcatggttagtggatgattttcccgctcggtgcgagtagtggtgcaataccagagacttttctatt
gccggatcaggacattgtgggacttttctggcaagtccgtggagaagccgctgctttgcgaagcacttctgttgtggttaatttaca
ggttggtgcttgtgcttttccagttgctcttatagtgccggtatctttgtaagcaagcgagttgtttatttgtctggtggatgacgcatcttc
cgatatcgc
The EST329 cDNA is translated into the following amino acid sequence (SEQ ID NO: 4):
mateareenvymaklaeqaerydemveamekvaktvdteeltveernllsvayknvigarraswriissieqkeeskgnde
hvsaikeyrgkveselsticdsilklldthliptsssgeskvfylkmkgdyhrylaefktgaerkeaaeatllayksaqdialtelapth
pirlglalnfsvfyyeilnspdractlakqafdeaiaeldtlgeesykdstlimqllrdnltlwtsdmqdevgpevkdakvddaeh
cDNA sequence of EST 373 from P. patens (SEQ ID NO: 5):
atcccgggcgtgtgagtaccctcattgctcgcagcagcatcatcaggttgtactgctcgaagcgaacgtttattgaatggccacc
acaattgatcttgatgtgtgggtggacggttgcaataaactcttttagcagcgctagatggcgttttcttaggccaagctgagagtc
ataagcgagtcagtttttgggtgaccatcactgcttatcgattcgtgagaagcattccacttggaattgcggatggttagtcaagga
tagtgaattggatgatgtagatgatttttacccacacatgggctgctgctcggtctgcagttcggtcctatgcagcatcaggatgatg
cttttgcttctgccaggacttcaccgggtcataacgagtccggagaggtacaaccgagggttagatgttggtgagcatggttggg
cgagttgacacccttgtcctcaattcatccgtcgttttcgcaatctgctgttcctagttctgcatgcaagcttccgtttcgagagtgtgag
tgacaactgttctagatccctaaaggatcagatattcgggaactcaagggtgctgttgcaattttcgaaagatgtggatggggtac
aaccacgcgctagtgcgaggagcgacaagcaaaccgatgaggggaagcggagctcttgcagtcactgttcgtattagaatt
gaggattttagcaacagaaggtcttgtggatctaagtccctgcgtttggcgatggaagttggtctcatcagctgaaatcctttgtagt
cgctaaacggccgagtttagtgtctggcggaattgaccattctgcagcactccaaggtctttcagctgatatgaaacaattgaca
aatgaggtatgcaaatactgtgggttgcgagacaagttcacaagacatttgattcaggatatataaccccatgcatagattatcc
aagcgtcacttagcagggatatttcagttttagaacagaatttgctaattgggcgaagctcttcaagttgatagtttcatgaatttcca
ctcattactggagtctgcgccagtttttcgaagtatcaaggagagtggtcaaaatggcggcgttgatggttgagacgcccatagc
cttcgggcttacgatggcggtgtgtttggctttattcttctattgttggcgcattcggaagtttcgtaatcggctcacctccgtccaagtc
gcagccacgcctaatgaagtgaattcagggttgcagattggaatcaagcaggatgtgatcaaaaccttcccaactgtgatgact
aaggagctgaaaattgacatcaaggatgggcttcagtgcccgatatgtctggtcgagtacgaggaggcggaagtgctgcgaa
aacttccactctgcggccatgttttccacatacgttgcgtcgactcctggctagaaaagcaagtcacttgtcctgtttgccgcattgtt
ctcgcgggagtttccaagttatcacttcgaactaaccgccagcaaaactatcttaatcactacagatttccctccagcccccgctct
gtaaccgtagaggtggctggcaacatacccgcatgggttcttgtcaatcgacctctgcccttgccaccagccattcctgagcgcc
cctcggtggacagcgtcacctctctagaatccagccccttggacattgatgtgcagccttcagccaatttcggcatgaccggcga
gtctccactcctcattcctcacgatgcaggatggggagctatctacctgcagaggagtcatggcgcactgagctttaaggcgcg
aacaggcgcagacatcgcaatcgaaaccaaagagtgcgtcgatcattcttccataagcgagaggtggatgacagagtcgttc
tcttttggcatctccacctgcgaggacgtgtcttcgacaagatctagccataatgtgtggcaagctgactcgactacacgccattct
tcgtggagctcacactcccacaactcattgtgtgatatcaaccaacccacgatgaagaattgggagtcggaggaagtgtttgag
tcgctagccacccatcaccagcccttgacgatgtccccagagcgctgctcctttgagtttctgcccatcatcacaggcactgaag
gtgactgcattttgaagcacaattcttatgcgccgaaaccagaaagaactgagatcggttcaagccctcactcttactcccagct
ctgaatttttcctcccgaattctggagaaccatctcttcaccacattagtgcactccgcaaatttcttcatggtcatgactgttggaagc
attcatttttcgggagggcggagtgcaccgctggttttacgtgtctcgcaacgaaggtttagaaggggactgtcggagaagattg
gtttgctcgaaaagagttgctccgttgaagaagcacttttacgggacggaatcccaaaccgaaaataaggttcaaattttaggc
agagtagatggtaacaaactgtacattcacactgtggcttaaggaatcaccgccggaatgtagtaatcttgtaaataatcaccc
agccgtgatcttagaggcgttaacgc
The EST373 cDNA is translated into the following amino acid sequence (SEQ ID NO: 6):
maalmvetpiafgltmavclalffycwrirkfrnrltsvqvaatpnevnsglqigikqdviktfptvmtkelkidikdglqcpiclveye
eaevlrklplcghvfhircvdswlekqvtcpvcrivlagvsklslrtnrqqnylnhyrfpssprsvtvevagnipawvlvnrplplpp
aiperpsvdsvtslesspldidvqpsanfgmtgesplliphdagwgaiylqrshgalsfkartgadiaietkecvdhssiserwm
tesfsfgistcedvsstrsshnvwqadsttrhsswsshshnslcdinqptmknweseevfeslathhqpltmspercsfeflpiit
gtegdcilkhnsyapkperteigssphsysql
cDNA sequence of HV62561245 from barley (SEQ ID NO: 7):
gcgagggggaaacgatgatgttcgggtcggggatgaatctcctcagcgcggcgctcggcttcggcatgaccgccgtcttcgtc
gcgttcgtctgcgcgcggttcatctgctgccgcgcccggggcgcgggcgacggcgccccgccgccggtggactttgacgttga
cttcccggcagatctcgaacgcccggtggaggatgctcattgtgggttggagcctttggttattgctgcaattcctattatgaagtac
tccgaggaattatattcaaaggatgatgcccagtgctccatatgtctaagtgaatacactgagaaagagcttctaagaatcattcc
gacatgtcggcataactttcaccgttcctgcttagatttatggttgcagaaacagactacttgcccaatatgccgggtctcgttaaaa
gagctgcctagcagaaaagctgctataacaccttcatgtagcaaccctcaagtgtgccctcgcactgagaactctgttaatcca
gcacctgactggctcctccctgttcatcattctcacagaggtcaacaaagtggtttagacacacaaggatcagtagaagtgatta
ttgagatacgccaataagcacagcatgaggttgctatggaagagagcaaaatgggaatatgtaataggtttcctgcctcattgc
attgttgcagcaccctaactggattggcattgtatgccacctcgttgcaggtaatgtgtaaacatttgttgtacatttcacattgtagat
aagcatattgtgttatgacacataaatactttcaatgttcttttctaatgcactgtatattgtaaaaatggtaaggaaatattggatgtta
gataaattcctg
The HV62561245 cDNA is translated into the following amino acid sequence (SEQ ID NO: 8):
mmfgsgmnllsaalgfgmtavfvafvcarficcrargagdgapppvdfdvdfpadlerpvedahcgleplviaaipimkysee
lyskddaqcsiclseytekellriiptcrhnfhrscldlwlqkqttcpicrvslkelpsrkaaitpscsnpqvcprtensvnpapdwllpvhhshrgqqsgl
dtqgsveviieirq
cDNA sequence of BN43173847 from canola (SEQ ID NO: 9):
ctctctccctctcaatctctcattcgccaccatcttcaaactcatgaactccaacgaccaatatccaatgggcaggcccgacgaa
accacctccggctcttctcgaacctacgccatgagcgggaagatcatgctgagcgccatcgtcatcctcttcttcgtcgtcatccta
atggtcttcctccacctctacgcccgctggtacctcctccgcgctcgccgccgtcatttccgccgccgcagccgtaaccgtcgctc
cacgatggttttcttcgccgcggatccttccgccgccgccgccgcctcgcgcggcctcgatcccgcggtgatcaagtctctcccc
gttttcgctttctccgagttgactcacaaagatctgaccgagtgcgccgtttgcctctccgagttcgaggaaggcgagtcgggtcg
ggttttgcccggttgcaagcatacgtttcatgttgactgtatagatatgtggtttcattctcattccacgtgtcctctctgccgctctctcgt
cgagcctcccgtggaggagcaagttgcgatcacgatttctcctgaaccggtttctgttgcaattgaaccgggttcgagctctggatt
gagaaaaccggcggcgattgaggtgccgaggaggaacttcagtgaatttgacgatcggaactcgccggcgaatcactcgttt
aggtcgccgatgagtcgtatgttatctttcactcggatgctgagcagaggaaactcctcgtcgcccatagccggagctccgcctc
aatctccgtcgtctaactgccggatagcgatgactgagtcagatatagagcgtggaggagaagagactaggtgagctattggt
cggaaagtaaaaactataaattttattacaggattgataaagtcaactagcctttgccgacggttgatttaagctccagtaacacg
ttgcgtggtctgaacgaatcttattcaccgagtgtttacttgtgttagtttagatagaattgtctgaagatgtacataaaattgtcagttgt
cgatgatgttatattgaatcttttttttccatttgtttttattcccagtctctatagactctttatgtaataccaccaattcaatggtcatgaaatcatgat
agagacttaacctg
The BN43173847cDNA is translated into the following amino acid sequence (SEQ ID NO: 10):
mnsndqypmgrpdettsgssrtyamsgkimlsaivilffvvilmvflhlyarwyllrarrrhfrrrsrnrrstmvffaadpsaaaaas
rgldpavikslpvfafselthkdltecavclsefeegesgrvlpgckhtfhvdcidmwfhshstcplcrslveppveeqvaitispe
pvsvaiepgsssglrkpaaievprrnfsefddrnspanhsfrspmsrmlsftrmlsrgnssspiagappqspssncriamtes
dierggeetr
cDNA sequence of BN46735603 from canola (SEQ ID NO: 11):
tttcacccaactctctctctctcagttcccactcgtgatccgaaagcatgagtcttagagacccgaatccagtaactaacacaccc
ggatccttttcggatccaggcgggttcgctataaacagcagaatcatgttcaccgccataatcataatcatattcttcgtcattctcat
ggtctctcttcacctctactctcgttgctacctccaccgctctcgccgtttccacatccgccgcttaaaccgtagtagacgcgccgcc
gccgctatgaccttcttcgccgatccttcctcctccacctccgaggtcaccactcgcggtctcgacccctccgtcgtcaaatctcttc
ccactttcacgttctccgccgcagccgccccggacgcgatcgagtgtgcggtttgcctctcggagtttgaggagagcgaaccgg
gtcgggttttgcccaattgcaagcacgcgtttcatgttgagtgcattgatatgtggtttctttctcattcctcttgtcctctgtgccgatcgc
tcgtcgaacctatcgccggagttgtaaaaactgcggcggaggaagtcgcgatttcgatttctgacccggtttcaggcgacacaa
acgacgttataggagctgggacttccgatcatgaagattccagggggaaaccggcggcgattgaagtctcaacgaggaatct
cggagaatcggagaacgagttgagtcggagtaactcgtttaagtcacgggtgatatcttccacgcggattttcagcaaagaac
ggagaagcgcttcgtcgtcttcttctatcgggttccctccgcctccggtctctagcatgccgatgacggagttagatatcgagtctg
gaggagaagagcctcgttgactttaagacgctaaatttttactgctacgtggacgtgtatgatttgttataaatgtttccttgtttagag
ctaagatgcggagatgaaataattctttgttagggcatcagcattgggacttcttaagcccatttcttagtaaatttgggtcgaaattt
aaatcaaaaaggctggatatgtttgg
The BN46735603 cDNA is translated into the following amino acid sequence (SEQ ID NO: 12):
mslrdpnpvtntpgsfsdpggfainsrimftaiiiiiffvilmvslhlysrcylhrsrrfhirrlnrsrraaaamtffadpssstsevttrgld
psvvkslptftfsaaaapdaiecavclsefeesepgrvlpnckhafhvecidmwflshsscplcrslvepiagvvktaaeevaisi
sdpvsgdtndvigagtsdhedsrgkpaaievstrnlgesenelsrsnsfksrvisstrifskerrsasssssigfppppvssmpm
teldiesggeepr
cDNA sequence of GM52504443 from soybean (SEQ ID NO: 13):
cctgccaccaaccaaaaccaatcctattacaacaagttcagcccttccatggccatcataatcgtcatcctcatcgccgccctctt
tctaatgggcttcttctccatctacatccgccactgctccgactccccctccgccagcatccgcaatctcgccgccgccactggac
gctcacggcgcggcacccgcggcctcgagcaggcggtgatcgacaccttcccgacgctggagtactcggcggtgaagatcc
acaagctgggaaagggaactctggagtgcgctgtgtgcttgaacgagttcgaggacaccgaaacgctgcgtttaatccccaa
gtgtgaccacgtgttccaccccgagtgcatcgacgagtggctagcttcccacaccacttgccccgtttgccgcgccaacctcgtc
cctcagcccggcgagtccgtccacggaatcccaatcctcaacgctcctgaggacatcgaggcccaacacgaagcccaaaa
cgacctcgtcgagcccgaacagcaacagcaagaccccaagcctcccgttcccactgaacctcaagtgctgtcattaaacca
gacgctgaaccggaaccgcaccagaggctcccggtcgggccggccgcggcgattcccgcggtctcactcgaccggtcattc
tttagtcctgccgggcgaagacactgaacggttcactttgcggcttcccgaggaagttagaaagcagatattgcagaacccgc
aactgcatcgcgcgagaagcctcgttatcttaccgagagaaggtagttcgcggcgggggtatcgaaccggtgaaggaagta
gcagagggagatcgtcgaggcggttggaccgggggtttaagtcggaccggtgggttttcaccatggcgccgccttttttggtga
gagcgtcgtcgattaggtcgcccagggtggccaataacggtggcgaaggaacttccgctgctgcgtctttgcctccgccgcctg
ctgtggagtctgtttgagttttgattcccccttctgcaagatttcaatattttattgtatttaccaattattttttgctgccacgatttttttacgctagaa
tttgtaagatgtgtataatatttggcacacttgttttgcgtttgaagataaataactgaaatcctgaatcacgatagattcttaa
atcataatcttggtcatcagttcagatatgaat
The GM52504443 cDNA is translated into the following amino acid sequence (SEQ ID NO: 14):
maiiiviliaalflmgffsiyirhcsdspsasirnlaaatgrsrrgtrgleqavidtfptleysavkihklgkgtlecavclnefedtetlrlip
kcdhvfhpecidewlashttcpvcranlvpqpgesvhgipilnapedieaqheaqndlvepeqqqqdpkppvptepqvlsln
qtlnrnrtrgsrsgrprrfprshstghslvlpgedterftlrlpeevrkqilqnpqlhrarslvilpregssrrgyrtgegssrgrssrrldrg
fksdrwvftmappflvrassirsprvannggegtsaaaslppppavesv
cDNA sequence of GM47122590 from soybean (SEQ ID NO: 15):
gtgatgtctgagtgtggctgttccgagtcagacccttcgtgtggttgttggtcgagcagcagcagcagatctgtggcctcaactga
actgaagctgtaccgagcattcatcttctgtgttcccatcttcttcactctcattctcctctttctcttctatctcttctacctccgaccgcga
actaggctccattggatttcacactttcgccttcccagcaacaacaaccgcaataatgccatctccacattgggtttgggcttgaac
aaagaacttagagagatgctgcccattattgtctacaaggaaagcttctccgtcaaagatactcaatgctcagtgtgccttttgga
ctaccaggcagaggataggctgcaacaaatacctgcatgtggccatacatttcatatgagctgcattgatctttggcttgccaccc
ataccacctgtcctctctgccgcttctccctactaaccactgctaaatcttcaacgcaggcatccgatatgcagaacaatgaaga
aacacaagccatggaattctctgaatcaacatctcctagggatctagaaaccaatgtcttccaaaatgtctctggagaagttgcc
atcagcactcactgcattgatgttgaagggcaaaatgtgcaaaacaatcaataggagcatgatgatgcaaaactctttcaggtg
tatcaagttgataatcaattctactatcaaaatgatgaaatccagatatattgacaaacttatcccttccaactcagttgaatgaagc
ctccagagtgtgcgcagcaactgcacagattgatacttcggcaagaaatgtcttcattcggggaactacagctttgatggtacatt
tgaattgactcatcattattgtaacttatggtaccctgaatgtgtcttttaagcattctaattttggttaatgtacctaagatagtttacatc
acaagtgaaaagtattttatg
The GM47122590 cDNA is translated into the following amino acid sequence (SEQ ID NO: 16):
msecgcsesdpscgcwsssssrsvastelklyrafifcvpifftlillflfylfylrprtrlhwishfrlpsnnnrnnaistlglglnkelrem
lpiivykesfsvkdtqcsvclldyqaedrlqqipacghtfhmscidlwlathttcplcrfsllttaksstqasdmqnneetqamefse
stsprdletnvfqnvsgevaisthcidvegqnvqnnq
This is the cDNA sequence of GM52750153 from soybean (SEQ ID NO: 17):
ggtaccaatttggtgaccacggtcattgggtttgggatgagtgccactttcattgtgtttgtgtgcaccagaatcatttgtgggaggct
aagagggggtgttgaatctcggatgatgtacgagattgaatcaagaattgatatggaacagccagaacatcatgttaatgacc
ctgaatccgatcctgttcttcttgatgcaatccctactttgaagttcaaccaagaggctttcagttcccttgaacacacacagtgtgta
atatgtttggcagattacagagaaagagaagtattgcgcatcatgcccaaatgtggccacacttttcatctttcttgcattgatatatg
gctgaggaaacaatccacctgtccagtatgccgtctgccgttgaaaaactcttccgaaacgaaacatgtgagacctgtgacattt
accatgagccaatcccttgacgagtctcacacatcagacagaaacgatgatattgagagatatgttgaacctacacctactgc
agccagtaactctttacaaccaacttcaggagaacaagaagcaaggcaatgatcttagagaactaaaggggttgttctgctca
aaaagagaagaatgtagaatttctgcttctatagaggaatgcttctaattatagattggattcaaattctttgtctgtaatatggccttc
atattcacttggtggtgtaaatatgtttccttttgtagcatatgcgggccaaggttttggtggaatttcttgcataccgatttgaagttctttt
gtctatggtatcgcttactcaagcaagcacactgctcttgttaatgcttaacagattaaacaaatggttgattac
This cDNA is translated into the following amino acid sequence (SEQ ID NO: 18):
msatfivfvctriicgrlrggvesrmmyeiesridmeqpehhvndpesdpvlldaiptlkfnqeafsslehtqcvicladyrerevlr
impkcghtfhlscidiwlrkqstcpvcrlplknssetkhvrpvtftmsqsldeshtsdrnddieryveptptaasnslqptsgeqearq
cDNA sequence of EST 548 from P. patens (SEQ ID NO: 19):
atcccgggagtggcaggctgtaactagcgtcatggccgcaggtggatcaagagcccgagccgattacgattaccccatcaag
ttgctgttgattggcgacagtggggttgggaaatcttgtcttctccttcgtttctcggatgactcctttactacaagtttcatcaccacaat
agggattgacttcaagatacggaccatcgagctggatgggaagcgcatcaagcttcagatatgggacacggctggacaaga
acgtttccgcacaatcacaacagcttactacagaggtgccatgggaatattgctggtatacgatgtaacggacgaatcttcattta
acaatattcggaactggatcaggaacatcgagcagcatgcatctgacaatgtgaacaagatcttggttggaaacaaagctgat
atggacgagagcaaaagagctgtcccaactgccaaaggtcaagccctagctgatgaatatggcatcaagttttttgaaactag
cgctaaaacaaacatgaacgtggaagatgttttcttcacaattgcaagggacatcaaacagaggttggctgagactgattcga
agcctgaggctgctaagaatgcaaagccagatgtcaagcttcttgcaggaaattctcagcaaaagccagcttctagttcctgct
gctcgtagctgaaagcttatgttgagacatttgtctggtaagcttttggatctattccgagtaaaggctgtctgagctcgc
The EST 548 cDNA is translated into the following amino acid sequence (SEQ ID NO: 20):
maaggsraradydypikllligdsgvgksclllrfsddsfttsfittigidfkirtieldgkriklqiwdtagqerfrtittayyrgamgillvyd
vtdessfnnirnwirnieqhasdnvnkilvgnkadmdeskravptakgqaladeygikffetsaktnmnvedvfftiardikqrla
etdskpeaaknakpdvkllagnsqqkpasssccs
cDNA sequence of GM50181682 from soybean (SEQ ID NO: 21):
ggaagggaaggaggagagggagagggagagagaaagaaaggtgaattggattgcatctctctctgtgtgttggaagaggg
gaatcgtagatctgatttctttctttctttttaataattttgtgatcagaattattgagctgaacaaaagacaatgggattgtgggaagct
tttctcaattggcttcgcagcctttttttcaagcaggaaatggagttatctctaataggacttcagaatgctgggaagacttcccttgta
aatgtagttgctaccggtggatatagtgaggacatgattccaactgtgggattcaatatgaggaaagtgacaaaagggaatgtt
acaataaagttatgggatcttggagggcaacctaggttccgcagcatgtgggaacgttactgtcgtgccgtttctgctattgtttatgt
tgttgatgctgccgatccagataaccttagcatatcaagaagtgagcttcatgatttgctgagcaaaccatcattgggtggcatcc
ctctgttggtattggggaacaagattgacaaagcgggggctctgtctaaacaagcattgactgaccaaatggatttgaagtcaat
tactgacagggaagtttgctgcttcatgatctcgtgcaaaaactcgaccaacatcgactctgttattgactggcttgtaaagcattcc
aaatcaaagagctgagagcctactttctgttttgaactctagtgtaatttatgggtgacacattttctggatttactagaggcatttgca
tgtctaactcggttgctgattgatttgtttttcccttttgtcagatgctttgtaatataatatcacatcattcttgtccaatagggagttaaacggg
The GM50181682 cDNA is translated into the following amino acid sequence (SEQ ID NO: 22):
mglweaflnwlrslffkqemelsliglqnagktslvnvvatggysedmiptvgfnmrkvtkgnvtiklwdlggqprfrsmwerycr
avsaivyvvdaadpdnlsisrselhdllskpslggipllvlgnkidkagalskqaltdqmdlksitdrevccfmiscknstnidsvid
wlvkhsksks
cDNA sequence of HV62638446 from barley (SEQ ID NO: 23):
ccggctccgacttcggccagaggaaggaaggcaggcaagggcggggacgatcgagccttccccgaaccccgcgcgcat
cccataaccttccactagccgttccattctcatcctcttcggcggccgaccagccggccagattctcctgatccagggttatgggtc
aggccttccgcaagctcttcgatgccttcttcggcaacaaggagatgcgggtggtgatgcttgggttggatgcagccggtaaaa
ccaccatactctacaagctacacattggcgaagtactctccaccgttcccactattggcttcaatgttgagaaggttcagtacaag
aatgtagtattcactgtgtgggacgtgggtggccaggagaaattgaggcccttgtggaggcactacttcaacaacacagatgct
ctgatctatgtggtcgattccctcgacagggatagaattggaagagccagggctgaatttcaggccataatcaatgacccgtttat
gctcaacagtgtattattggtgtttgctaacaagcaagacatgaggggagcaatgactccgatggaagtatgcgagggtcttggt
ctgtacgacctgaacaatcgtatctggcatatccaaggtacctgtgctcttaaaggcgatggcctgtatgaaggcttggactggct
agcgacgaccctggatgaaatgcgagctacagggcggttagcttcgacatcggcgtaaagagtaacagggaaggaccgtc
tgtgtttcttggcccctcatttttcctttttgtgtctgccctgtggccgctttttgatgtgttcgacagatttgttgtagtatgaatgattcacaa
gaggagatgcgttttctgaagagggggtcatcctcttagttggaggcgcatatatattctgttctactctaggattgtgggatgtaaat
actgatgtttctactgatggcatgacacgcttaatatttgtggtttagtctgaag
The HV62638446 cDNA is translated into the following amino acid sequence (SEQ ID NO: 24):
mgqafrklfdaffgnkemrvvmlgldaagkttilyklhigevlstvptigfnvekvqyknvvftvwdvggqeklrplwrhyfnntda
liyvvdsldrdrigraraefqaiindpfmlnsvllvfankqdmrgamtpmevceglglydlnnriwhiqgtcalkgdglyegldwl
attldemratgrlastsa
cDNA sequence of TA56528531 from wheat (SEQ ID NO: 25):
acggacgaagcggagatcgatcggacgaacgccgccgccgcatcggagcacgcgcgcgcgcgagcgaagccgtcccc
gcctcgctcggcctgggagttagggcgcgatggcggcgccgccggctagggctcgggccgactacgactacctcatcaagct
cctcctcatcggcgacagcggtgttgggaaaagttgtctgcttctgcggttctcagatggctccttcaccactagcttcatcaccact
attggtattgacttcaagataaggactgttgagttggatggtaagcggattaagttgcagatctgggatactgctggccaagaac
gctttcggactataactactgcctactacaggggagcgatgggcattttacttgtttatgatgtcacggacgaggcgtcattcaata
acatcagaaattggatcaaaaacattgaacagcatgcttcagataacgtgagcaaaattttggtggggaacaaagcggatat
ggatgaaagcaaaagggctgttcccacttcaaagggccaggccctggccgatgaatacgggatccagttctttgaagcgagt
gcaaagacaaacatgaatgtcgagcaggttttcttctctatagcaagagacatcaaacagagactctcggaggcagattccaa
gactgagggggggactatcaagatcaacacggagggtgatgccagtgcagcagcaggacagaagtcggcttgctgtgggt
cttgaaccgtcgtcgtcgctacggaaaaaaaaagatagttgcgacacggtgcttgtaattcttgtcattccattctttgcctgctggtt
tcgttgtgttatttaagttatcgctgttgttaggatttggacaaattggtgttacgtcagcaattacttgcagtatcggtgg
The TA56528531 cDNA is translated into the following amino acid sequence (SEQ ID NO: 26):
maappararadydylikllligdsgvgksclllrfsdgsfttsfittigidfkirtveldgkriklqiwdtagqerfrtittayyrgamgillvyd
vtdeasfnnirnwiknieqhasdnvskilvgnkadmdeskravptskgqaladeygiqffeasaktnmnveqvffsiardikqr
lseadskteggtikintegdasaaagqksaccgs
cDNA sequence of HV62624858 from barley (SEQ ID NO: 27):
caaatcgccgaagcaactgataggagagaggaagtgggggagagatcttcgtcttcaccactcgcgcgcgcaagctcgctc
gctccagatctcccccttccatcgtagatcccacgaccgcaagccgccgcgtccccgacgaaaccctagctcgcgcccctcc
gccgcgtaggggcgccgccatgggcatcgtgttcacgcggctcttctcgtcagtattcggaaaccgcgaggctcgcatcctcgt
cctcggccttgacaatgccggcaagactactatcctctatcggctgcagatgggggaggtcgtctccacgatcccaacaatcgg
cttcaacgtggagacggtgcagtacaataacatcaagttccaagtttgggatctcggtggtcaaacaagcataaggccgtactg
gagatgctactttccaaacactcaggctatcatatatgttgttgattcaagtgatactgataggctggtaactgcaaaagaagaatt
tcattctatccttgaggaggatgagctgaaaggtgcggttgtccttgtatatgcaaataaacaggaccttccaggtgcacttgatg
atgctgccataactgaatcattagaacttcacaagattaagagccgccaatgggcaattttcaaaacatctgctataaaagggg
agggcctttttgaaggcttgaattggctcagtaacgcactcaagtccggaagcagctaatgcaggctccattccgcgaatcattg
cttgatggtaaggaacagggacgatgacatccttctcactagtctgcgcgaaaatcacattctctttatttaactcggaagttatac
acaatcagttatctgtagagtgcttgttgaagtttccagatacaacaccaggtgtacccatatcgggagcaagaatatatttgtag
aacatactgagcagacttatggtttgaaatctatggcttcaccgcg
The HV62624858 cDNA is translated into the following amino acid sequence (SEQ ID NO: 28):
mgivftrlfssvfgnrearilvlgldnagkttilyrlqmgevvstiptigfnvetvqynnikfqvwdlggqtsirpywrcyfpntqaiiyvv
dssdtdrlvtakeefhsileedelkgavvlvyankqdlpgalddaaiteslelhkiksrqwaifktsaikgeglfeglnwlsnalksgss
cDNA sequence of LU61640267 from linseed (SEQ ID NO: 29):
ctcgcgcctcccttctcttcttcgagatccaaagctagggcaaaaaacctttcccacaacacctcctccttcatttcgttctctgtctgt
agtttcaagatgggtctatcattcaccaagctgttcagccggctatttgccaaaaaagagatgcggattctgatggtgggtctcgat
gcagctggtaagactacaatcttgtacaagctcaagcttggagagatcgtgacaaccattcccaccattggattcaatgttgaga
ccgtggaatacaagaacatcagcttcactgtctgggatgttggaggtcaagacaagatccgtccattgtggagacactacttcc
aaaacactcaaggactgatctttgtcgttgatagcaacgatcgcgatcgtgtggtcgaggctagagatgaacttcatcgcatgttg
aatgaggatgagttgagggatgcagttctgctagtctttgccaacaaacaggatctcccgaatgccatgaatgcagctgagatc
acggacaagcttggccttaattcccttcgtcagcgccactggtacatccagagcacctgcgctacctctggtgaaggactctacg
agggactcgactggctgtccaacaacattgccaacaaggcatagaggactgtggtagacttcacgaagccttatgtaactgctt
cgatactgccgctagcgcgaacccataatatgatgtttttcgtgtttgttttgaggggtatgtcgatgtatcctgtaatcgtttgcaagtg
atgttggtaattctatctttttgtagattctcaaaataataatctttcatacgtattgttaaatatgattctgtaacgtgactcacaagttac
ctcttt
The LU61640267 cDNA is translated into the following amino acid sequence (SEQ ID NO: 30):
mglsftklfsrlfakkemrilmvgldaagkttilyklklgeivttiptigfnvetveyknisftvwdvggqdkirplwrhyfqntqglifvvd
sndrdrvveardelhrmlnedelrdavllvfankqdlpnamnaaeitdklglnslrqrhwyiqstcatsgeglyegldwlsnnianka
cDNA sequence of LU61872929 from linseed (SEQ ID NO: 31):
agcagcagggcgcaccggtcggccggccctttcccgatatgttcctattcgactggttctatggaattctcgcatctcttgggctatg
gcagaaagaggccaagatcctcttcttgggtctcgacaacgccggcaagaccactcttcttcacatgttgaaagacgagagac
tagtgcaacatcagccgacccagcatcctacttcagaggagttgagtattggcaaaatcaagttcaaagcttttgatttgggcgg
ccatcagatcgctcgccgcgtctggaaagactattatgccaaggttgatgccgtggtctaccttgttgatgcctacgacaaggag
aggtttgcagagtcgaagaaggagctggacgccctcttgtcagacgagggccttaccagtgttccattcctgatcctaggcaac
aaaatcgacatcccctatgcagcatcggaagacgagctccggtaccatctagggctgtcgaatttcacaaccggaaagggca
aggtgaacctcacggactccaacgtccggcctcttgaggttttcatgtgcagcattgtccggaagatgggttacggagaaggctt
caagtggctctctcagtacatcaagtagaggaattatatcaagatataatagaagatggggttattcagtactttctcctcccctca
gctgttctgtatttttgtactggagcttatttcctcatgcccttgcccattactgtttttgtttctgggtttatcgatgttttgttttttgcaagtcagt
tagatacaattagattggaagaatgggtattcttttgctgctgttatggataaactggattggtgtaaggagattaagcaacttgggagagcc
The LU61872929 cDNA is translated into the following amino acid sequence (SEQ ID NO: 32):
mflfdwfygilaslglwqkeakilflgldnagkttllhmlkderlvqhqptqhptseelsigkikfkafdlgghqiarrvwkdyyakvd
avvylvdaydkerfaeskkeldallsdegltsvpflilgnkidipyaasedelryhlglsnfttgkgkvnltdsnvrplevfmcsivrk
mgygegfkwlsqyik
cDNA sequence of LU61896092 from linseed (SEQ ID NO: 33):
cccgcctctgctcatacacgattaccacgattactaagttatcttttcattatctctttccctcgcccacccgctgcacctttcgatcattc
tcccgaatcaacttggattggtaatttttgctttcgatccgtttctcaagggggagtagaagcagaagatgggagcattcatgtcta
gattttggttcatgatgtttccagctaaggagtacaagattgtggtggttggattggataatgcagggaagaccaccactctttaca
aattgcacttgggagaggtcgtcactactcaccctactgtcggtagcaatgtggaagaagttgtctacaagaacattcgtttcgag
gtgtgggaccttggaggacaagagaggctgaggacatcatgggcaacatattacagaggaacacatgccataatagtagtg
atagacagcacggatagagcaaggatttcgataatgaaggatgaactttttagactgattgggcatgacgaattgcagcagtc
ggttgtactggtatttgcaaacaaacaagatctaaaggacgccatgactcctgctgagataacagatgcactttcactccacag
catcaaaaatcacgactggcacatccaggcatgttgcgcactcaccggtgaaggcttgtacgacggccttggatggattgcac
agcgtgttactggcaaggccccaagttagaagtgaaagttggtgatgaggtggaggaaattatagagagcatcttctttcttgta
caccatctgattgtacttgttcatcaatttactgcaattgtgtttcttgcgactc
The LU61896092 cDNA is translated into the following amino acid sequence (SEQ ID NO: 34):
mgafmsrfwfmmfpakeykivvvgldnagktttlyklhlgevvtthptvgsnveevvyknirfevwdlggqerlrtswatyyrgt
haiivvidstdrarisimkdelfrlighdelqqsvvlvfankqdlkdamtpaeitdalslhsiknhdwhiqaccaltgeglydglgwi
aqrvtgkaps
cDNA sequence of LU61748785 from linseed (SEQ ID NO: 35):
agcaaatcactttcgattctcgcctttaggttttcaattgagttgattgagatagaggagccatgtttctgatcgattggttctacggag
ttctcgcatcgctcgggctgtggcagaaggaagccaagatcttgttcctcggcctcgataatgccgggaaaaccactctcctcca
catgttgaaagatgagaggctagtgcagcatcagccgactcagtacccgacttctgaagagctgagcattgggaaaatcaag
ttcaaagcttttgatcttggtggtcaccagattgctcgtagagtctggaaagattactatgctaaggtggacgccgtggtctacttggt
cgatgcattcgacaaggaaagattcgcagagtccaagaaggaactcgatgcactcctctccgacgagtcactctccaccgtc
cctttcctgatacttgggaacaagatcgacataccatatgctgcctcggaagacgagttgcgttaccacttggggctcacaaactt
caccaccggcaagggcaaggtgaacttgagtgacacgaatgtccgccccctcgaggtgttcatgtgcagcatcgtccgcaaa
atggggtatggcgaagggttcaagtggatgtctcagtacatcaactagaccgtattgtagtgtgttttgtttttgtcttcagacattctc
aatggtatttttctacttgttatggtgttcttgttctgagtctggtgttaaaaaatatgtaatatacataaacctgattagagtttggtttttcta
ctgtattgtctgtatcatattttcctactatccaatgcttatagtctttcaagatcttatatctcg
The LU61748785 cDNA is translated into the following amino acid sequence (SEQ ID NO: 36):
mflidwfygvlaslglwqkeakilflgldnagkttllhmlkderlvqhqptqyptseelsigkikfkafdlgghqiarrvwkdyyakvd
avvylvdafdkerfaeskkeldallsdeslstvpflilgnkidipyaasedelryhlgltnfttgkgkvnlsdtnvrplevfmcsivrkm
gygegfkwmsqyin
cDNA sequence of OS34706416 from rice (SEQ ID NO: 37):
cctacccaaaacaaaacttcaatttctgtttcagttcgcggagatcaatattttatctaggtccatcgtcgatagaagatacgagaa
accaaaggcaatgtttttgtgggattggttttatgggattctagcgtcgctcgggctgtggcagaaggaggccaagatcttattcttg
ggcctcgataacgctggcaaaactaccttgcttcacatgctcaaagatgagagattagtccagcatcagcctacccagtatcct
acatcggaggagttgagtattgggaagatcaagtttaaagcttttgatctagggggtcatcagattgctcgaagagtttggaaag
attactatgcccaggtggatgcagtggtgtacttggttgatgcttatgacaaggagagatttgctgagtcaaaaaaagagctgga
tgctctactctctgatgaatctttagccagtgtcccttttcttgtccttgggaacaagatagatattccatatgctgcctcagaagaaga
attgcgctaccatttgggcctgactaacttcaccacaggcaagggtaaggtaaacttggccgactcaaatgtccgtcccatgga
ggtattcatgtgcagtattgtgaagaaaatgggttatggggatggtttcaaatgggtttcccagtacatcaaatagtcccttagcaa
gagatggcttggtacctcatttctagaagtttgtttctctagttgagatttggaggtgttgttgggacaaaattgctgttaaagaaattg
cagtatatttcaacttttatttatataaaatgactgggaaccttctcctgttttccccaccctcctacactgtcgatgatgtgctgagcaa
atttcagttgatttgtggtgattgatgattttttaggtgaaaaattgaggtggcccgaattattaggcatgctg
The OS34706416 cDNA is translated into the following amino acid sequence (SEQ ID NO: 38):
mflwdwfygilaslglwqkeakilflgldnagkttllhmlkderlvqhqptqyptseelsigkikfkafdlgghqiarrvwkdyyaqv
davvylvdaydkerfaeskkeldallsdeslasvpflvlgnkidipyaaseeelryhlgltnfttgkgkvnladsnvrpmevfmcsi
vkkmgygdgfkwvsqyik
cDNA sequence of GM49750953 from soybean (SEQ ID NO: 39):
ccaaaacaaaacttcaatttctgtttcagttcgcggagatcaatattttatctaggtccatcgtcgatagaagatacgagaaacca
aaggcaatgtttttgtgggattggttttatgggattctagcgtcgctcgggctgtggcagaaggaggccaagatcttattcttgggcc
tcgataacgctggcaaaactaccttgcttcacatgctcaaagatgagagattagtccagcatcagcctacccagtatcctacatc
ggaggagttgagtattgggaagatcaagtttaaagcttttgatctagggggtcatcagattgctcgaagagtttggaaagattact
atgcccaggtggatgcagtggtgtacttggttgatgcttatgacaaggagagatttgctgagtcaaaaaaagagctggatgctct
actctctgatgaatctttagccagtgtcccttttcttgtccttgggaacaagatagatattccatatgctgcctcagaagaagaattgc
gctaccatttgggcctgactaacttcaccacaggcaagggtaaggtaaacttggccgactcaaatgtccgtcccatggaggtatt
catgtgcagtattgtgaagaaaatgggttatggggatggtttcaaatgggtttcccagtacatcaaatagtcccttagcaagagat
ggcttggtaactcatttctagaagtttgtttctctagttgagatttggaggtgttgttgggacaaaattgctgttaaagaaattgcagtat
atttcaacttttatttatataaaatgactgggaaccttctcctgttttcctc
The GM49750953 cDNA is translated into the following amino acid sequence (SEQ ID NO: 40):
mflwdwfygilaslglwqkeakilflgldnagkttllhmlkderlvqhqptqyptseelsigkikfkafdlgghqiarrvwkdyyaqv
davvylvdaydkerfaeskkeldallsdeslasvpflvlgnkidipyaaseeelryhlgltnfttgkgkvnladsnvrpmevfmcsi
vkkmgygdgfkwvsqyik
cDNA sequence of HA66696606 from sunflower (SEQ ID NO: 41):
ccaaattccacaactcacaacccccctttctctctttctccttcgatccctctccacatccacagggatcctacgcggcaaaaaaat
ggggctaacgttcacgaaactctttagtcggctgtttgccaagaaggagatgcggatcttgatggtgggtcttgatgcagctggta
agacgaccattttgtacaagctcaagcttggtgagatcgtgacaacgattcctaccattgggtttaacgtggagaccgtggagta
caaaaacatcagcttcaccgtctgggatgtcgggggtcaagacaagatccgtccgttatggaggcactacttccagaacacac
aaggtcttatctttgtggttgatagcaatgacagggatagagttgttgaggcaagagatgaattacataggatgttgaatgagga
cgagcttcgagatgcagtcttgcttgtgtttgctaacaaacaagatcttccaaatgcaatgaatgctgccgaaatcactgataagc
ttggccttcattcccttcgccaacgccactggtacatccagagcacctgtgcaacctcaggagagggactttacgagggtctcga
ttggctttccaataacatcgctaacaaggcataagatgaaacaagaccaaacctaatgtcgatcttggatgctgggagcttttgct
ttgctctgtgtgtttgttaatgggtagcaaatgtgtctacttatataatatttggctgtattgcagttactttttaaaagcattgtctaaagttt
gtaacagaggttaattttgattgttttattatatgatgatgatgtttcttaacc
The HA66696606 cDNA is translated into the following amino acid sequence (SEQ ID NO: 42):
mgltftklfsrlfakkemrilmvgldaagkttilyklklgeivttiptigfnvetveyknisftvwdvggqdkirplwrhyfqntqglifvvd
sndrdrvveardelhrmlnedelrdavllvfankqdlpnamnaaeitdklglhslrqrhwyiqstcatsgeglyegldwlsnnianka
cDNA sequence of HA66783477 from sunflower (SEQ ID NO: 43):
actccaactgttacagaaataggtcagatccataaacataaccgcttgtgcaactccagatctgtgaacaaattcgatcaattctc
tcaattcaacgatgtttttgttcgattggttctacggcatccttgcgtcactcggtttatggcagaaggaagcgaagatcttgttccttg
gcctcgataacgccggtaaaacgacgttgcttcatatgttgaaagacgagagattagttcaacatcaaccgactcaacatccg
acgtcggaagaattgagtatagggaagattaagttcaaagcgtttgatttaggaggtcatcagattgctcgtagagtctggaagg
attattacgccaaggtggatgccgtagtgtatctagtagatgcatatgataaagaacggtttgccgaatcaaaaaaggaactag
atgcacttctttctgacgagaatctgtctgcagtcccctttctgattttaggaaacaagattgatataccatatgcagcctcagaaga
tgagctgcgttaccaccttggactgacaggggtcacgactggcaaagggaaggtaaatcttcaagattcaagcgtccgcccct
tggaggtatttatgtgcagcattgtgcgcaaaatgggttacggtgatggtttcaaatgggtctctcaatacatcaaatagtgggcgc
ctgagcaaatcgagtatcttatctgggaaataaaaaaggtaaggaagaatatggtgatttccccaatttgattttgtattcattctgt
aagagtgggattttgtttgtttgtgttggcatgtaaaattctgttagaccaaattgctagttgttttgtttg
The HA66783477 cDNA is translated into the following amino acid sequence (SEQ ID NO: 44):
mflfdwfygilaslglwqkeakilflgldnagkttllhmlkderlvqhqptqhptseelsigkikfkafdlgghqiarrvwkdyyakvd
avvylvdaydkerfaeskkeldallsdenlsavpflilgnkidipyaasedelryhlgltgvttgkgkvnlqdssvrplevfmcsivrk
mgygdgfkwvsqyik
cDNA sequence of HA66705690 from sunflower (SEQ ID NO: 45):
ccaaacgaataaccttcacccttggatcactcgcccttgttatataccccctgcaatttctataccatgaatcacgaatatgattactt
gttcaagcttttgctgattggggattcgggagtcggcaaatcttgtctcctacttagatttgctgatgactcatatattgacagctacat
cagcacaattggtgtggactttaaaatccgcaccgttgagcaggatggaaaaaccattaagcttcaaatttgggacacagctg
gacaagaaaggttcaggacaattaccagtagctactaccgtggggcccatggcattatcatagtttacgatgttactgacctaga
cagtttcaacaacgttaagcaatggttgagtgaaattgaccgttatgcaagtgaaaatgtgaataaacttcttgttggaaacaaat
gtgaccttacagaaagtagagccgtgtcctatgatactgctaaggaatttgcggataacattggcattccgtttatggaaactagt
gccaaagatgctaccaatgttgagcaggctttcatggccatgtcctctgacatcaaaaacaggatggcaagtcagcctggggc
aaacaacacgaggccaccttctgtgcagctcaagggtcaacctgttggtcaaaagggcggttgctgctcatcttagaatacca
gtcttgcagctgtttgattataaagaatcaccatgaatccaactgtcattcaagttttttgctattttattttcatataattcccctataaaa
gctattatagtttttattatttcaagaatttaatttttttttttaaaattggttgtacaaatttgcaaaaactgtctgctgctagtgttgatttgctattc
ttt
The HA66705690 cDNA is translated into the following amino acid sequence (SEQ ID NO: 46):
mnheydylfkllligdsgvgksclllrfaddsyidsyistigvdfkirtveqdgktiklqiwdtagqerfrtitssyyrgahgiiivydvtdl
dsfnnvkqwlseidryasenvnkllvgnkcdltesravsydtakefadnigipfmetsakdatnveqafmamssdiknrmas
qpganntrppsvqlkgqpvgqkggccss
cDNA sequence of TA59921546 from wheat (SEQ ID NO: 47):
ccgaagttactctcttcgtcttgagcactcgcgcgcgcaagctcactcgctccagatctccccttaccatcgtgtagatctcacgcc
cccaagccgccacgcccccaacgagacctagctcgcgcccctccgccgcgtaggggcgccgccatgggcatcgtgttcac
gcggctcttctcgtcggtattcggaaaccgcgaggcccgcatcctcgtcctcggcctcgacaatgccggcaagactactatcctc
tatcggctgcagatgggggaggtcgtttccacgatcccaacgatcgggttcaacgtggagacggtgcagtacaataacatcaa
gttccaagtttgggatctcggtggtcaaacaagcatcaggccatactggagatgctactttccaaacactcaggctatcatatatg
ttgttgattcaagtgatactgataggctggtaactgcaaaagaagaatttcattccatccttgaggaggatgagctgaaaggtgc
ggttgttcttgtatatgcgaataaacaggaccttccaggtgcacttgatgatgctgccataactgaatcattagaacttcacaagatt
aagagccgccaatgggcaattttcaaaacatctgctataaaaggggaggggttttttgaaggcttgaactggctcagtaatgca
ctcaagtccggaggcagctaatgtaggaggcccagcctccattccgtgaatcattgcttgatggtaaggaacagggacgatga
cagccttctcgctagtctgcgtggaaatcagaatccctttattttaactctggaagttatacacaatcagttatctgtagagtgcttgtt
gaagtttccagacacaacactaggtgtaccatgtcgagagcaagaatatatttgtagaaaataccgagcaaacgattacggttt
gaaatag
The TA59921546 cDNA is translated into the following amino acid sequence (SEQ ID NO: 48):
mgivftrlfssvfgnrearilvlgldnagkttilyrlqmgevvstiptigfnvetvqynnikfqvwdlggqtsirpywrcyfpntqaiiyvv
dssdtdrlvtakeefhsileedelkgavvlvyankqdlpgalddaaiteslelhkiksrqwaifktsaikgegffeglnwlsnalksg
gs
cDNA sequence of HV62657638 from barley (SEQ ID NO: 49):
cccgccccctcgtctgccggtcggggatcagcaacagcgccgatcgaggggtaggacgaggaggaggaggcgggtgcgc
gcgacatggctgcgccgccggcgagggcccgggccgactacgactacctcatcaagctcctcctcatcggggacagcggtg
ttggcaagagttgcctccttctgcggttctctgatggctccttcactacgagctttattaccacgattggtattgactttaagatcagaa
caatagagctggatcagaaacgtattaagctacaaatatgggacacggctggtcaagaacggttccggactattaccactgcg
tattaccgtggagccatgggtatcctgcttgtttatgacgtcaccgacgagtcatctttcaacaacataaggaactggatccggaa
cattgagcagcatgcctctgacaacgtcaacaaaattttgattggcaacaaggctgatatggatgagagtaaaagggctgtac
ctactgcgaaggggcaagctttggccgatgaatatggcatcaagttctttgaaactagtgccaagacaaacctgaacgtggag
caggttttcttctccattgcccgcgacattaagcagaggcttgccgagaccgattccaagcctgaggacaaaacaatcaagatt
aacaaggcagaaggcggtgatgcgccggcagcttcgggatctgcctgctgtggctcttaagggatggatgattgagtgtgtcg
gtgatcattgtttatttgacatcattcggttcccgctgctgctgctgcttgtctgttataggaagaatgtcaatcaagaagaaaactatg
acttatgatacagatctggttgtacttatattcgcttcccattctttgaagcaactacccttgcctttgacgg
The HV62657638 cDNA is translated into the following amino acid sequence (SEQ ID NO: 50):
maappararadydylikllligdsgvgksclllrfsdgsfttsfittigidfkirtieldqkriklqiwdtagqerfrtittayyrgamgillvyd
vtdessfnnirnwirnieqhasdnvnkilignkadmdeskravptakgqaladeygikffetsaktnlnveqvffsiardikqrlae
tdskpedktikinkaeggdapaasgsaccgs
cDNA sequence of BN43540204 from Brassica (SEQ ID NO: 51):
gacacgcctaaccgtaacctccttttatttttttcttagaaaacttcttttttcctgggaaaaattcacgaatcaatcggaaaaaactca
cgaagagctcgagaaaccatgagcaacgagtacgattatctgttcaagcttctgttgatcggcgactcatccgtaggaaaatca
tgcctgcttcttcgattcgctgatgatgcgtacatcgacagttacataagtaccattggtgttgacttcaaaattaggacgattgagc
aggatgggaagacgattaagcttcaaatctgggatactgctgggcaggagcgtttcaggaccatcactagcagctactacag
aggagctcatggaatcattattgtgtatgactgtaccgagatggagagtttcaacaatgtgaagcagtggttgagtgagattgac
agatatgctaatgacagtgtttgcaagcttcttattggtaacaagaatgatatggttgaaagtaaagttgtttccaccgaaactgga
aaggccttagccgatgagctcggaataccctttctcgagacaagtgctaaggattctatcaacgtcgaacaggcattcttaacta
ttgctggcgagatcaagaagaaaatgggaagccagacgaatgcaaacaagacatctggaagtggaactgtccaaatgaa
aggtcagccaatccaacagaacaatggtggcggttgctgcggtcagtagttaagcaaagtgttatcaaaactatgtgagactttt
ttttttcttactatgtgctgtgaaaactaatggctgtctaaaacagtaacgctggaaactttgataccatgtcactctatgttcaatctat
ggtggtagttgcg
The BN43540204 cDNA is translated into the following amino acid sequence (SEQ ID NO: 52):
msneydylfkllligdssvgksclllrfaddayidsyistigvdfkirtieqdgktiklqiwdtagqerfrtitssyyrgahgiiivydctem
esfnnvkqwlseidryandsvckllignkndmveskvvstetgkaladelgipfletsakdsinveqafltiageikkkmgsqtn
anktsgsgtvqmkgqpiqqnngggccgq
cDNA sequence of BN45139744 from Brassica (SEQ ID NO: 53):
tccaccctccccccccagattttcctctgttcgctgtcatctaaagtcgaaaccaccatgaatcccgccgagtacgactaccttttca
agctcctgctcattggggattctggcgtgggcaagtcttgtctactgttgagattctctgatgattcgtatgtagaaagttacataagc
actattggagtcgattttaaaattcggactgtggagcaagacgggaagacgattaagctccaaatttgggacactgctggtcaa
gagcgcttcaggactattactagcagttattaccgtggcgcacatggaatcattattgtctacgacgtcacagatcaagaaagctt
taataatgtgaagcaatggttgagtgaaattgatcgttatgctagtgacaatgtgaacaaactcctagttggaaacaagtgtgatc
ttgctgaaaacagagccgttccatatgaaaccgcaaaggcttttgccgatgaaattggaattcctttcatggagactagtgcaaa
agatgctacaaacgtggaacaggctttcatggccatgtcggcatccatcaaagagagtatggcaagccaaccagctgggaa
cattgccagaccgccgacggtgcagatcagaggacagcctgttgcccaaaagaatggctgttgctcaacttgattgcctagca
atatccttttccgttcagtcttcgagtcctacaaccttaagccaaaattgttttctcttcagttcacttgtactttgtacgtcatttctggtctgt
aattaaggtcacttgtcctttggttggctgtttttctctttgcgtatcaacattttcgtaccaccacatttttgtggctgccttcagtgtatttat
atactgtcgttttgcttaacaatgtttattagat
The BN45139744 cDNA is translated into the following amino acid sequence (SEQ ID NO: 54):
mnpaeydylfkllligdsgvgksclllrfsddsyvesyistigvdfkirtveqdgktiklqiwdtagqerfrtitssyyrgahgiiivydvt
dqesfnnvkqwlseidryasdnvnkllvgnkcdlaenravpyetakafadeigipfmetsakdatnveqafmamsasikes
masqpagniarpptvqirgqpvaqkngccst
cDNA sequence of BN43613585 from Brassica (SEQ ID NO: 55):
tccgtcatttccattgatctctctcgttcttctctgctcatcactatcaccacggtcctcttctctgcctcgtttgatccgattcgatttcgatg
gcagctccacctgctaggggtagagccgattacgattacctcataaagcttctcctgatcggtgatagcggtgtgggcaaaagtt
gtttgctgttaaggttctctgatggctcattcaccactagcttcatcaccaccattgggtttgtattatctttaagaatctattagagacta
tggtgatgcatgatgtttcacactgactctctttggtgtttgtgtgttggcttataatgatgcagcattgattttaagattagaactattgag
cttgatactaaacgcatcaagctccagatttgggatactgctggtcaagaacgttttcgaaccatcaccactggttagtcagtgga
aattggattagagaggattaagagtcactagcagtctacttaatgctatggatgatgctttgaggatatttagtttttttttttttttttgaaa
actgataagtaccattgcagcttattaccgaggggcaatgggcattttgctggtctatgatgtcacagacgagtcatcctttaacag
taacttttgcttctgtctaagcattgacatcttttattttatttacatttttgctctgttctggacctgttttcttgaccttgttgcagatattagga
actggattcgtaatattgaacagcacgcttcggataatgttaataaaatcttggtagggaacaaagccgatatggatgagagca
agagggctgttccaacatcaaagggtcaagcacttgctgatgaatatggaatcaagttctttgaaacaagtgccaaaacaaat
ctaaatgtggaagaggttttcttctcgatagcaaaggacattaagcagagactcacagatactgactcgagagcagagcctgc
gacgattaggataagccaaacagaccaggctgctggagccggacaagccacgcagaagtctgcatgctgtggaacttaaa
agttactcaagttgaagtgaagtgcaaagaaaccagatttgtgccaaatcatttgtcttgtctttggtgcttttgtatttttttttctcttttga
tgattgttctaaatttgccatttttagtttagattcgatggccctatagctgattcagtggcttttgattgttaacacttttgctcacaactca
aaatctcttgcactctctgttaataaagcttttccctttgcagcac
The BN43613585 cDNA is translated into the following amino acid sequence (SEQ ID NO: 56):
mgillvydvtdessfnsnfcfclsidifyfiyifalfwtcfldlvadirnwirnieqhasdnvnkilvgnkadmdeskravptskgqala
deygikffetsaktnlnveevffsiakdikqrltdtdsraepatirisqtdqaagagqatqksaccgt
cDNA sequence of LU61965240 from linseed (SEQ ID NO: 57):
ttttccacccaatttctctcccaactccgattcgccggcgtagcttcgtccgcctccgacgagttcgagcccgatctccttaaccgcc
gacaacgtcatcatcatgaacactgaatacgattacttgttcaagcttttgcttattggagattctggagtcggcaaatcgtgtctgct
tttgagattcgctgatgattcgtaccttgacagctacatcagtaccataggagtcgatttcaaaatccgcactgtggagcaggatg
ggaagaccatcaaactccaaatttgggacacagcagggcaagagcgatttaggacgatcaccagcagttactacaggggt
gctcacgggatcattgttgtttatgatgtcacggaccaagagagtttcaacaacgtaaaacagtggctgaacgagatcgatcgc
tacgctagcgagcacgtgaacaagcttcttgtgggaaacaagagtgacctcactagcaacaaagtcgtttcgtatgaaacagg
gaaggcattagctgatgaactcggtatcccgttcatggagacgagtgccaagaacgcgtccaacgtagaagacgctttcatgg
ccatgtcagctgcaatcaagaccaggatggctagccagcccacgaacaatgccaagccaccgactgtccaaatccgtgga
gaaccggtcaaccagaagtcaggctgctgttcttcttgaacagcatggattgggatcgtacggtgatgttaatcgtgttcggctaat
ccttgtggcatgtaaacttggtttcaatattcttattggttttccatatgaacgacaggattattcgtttcgttttcgccttcctgtttttttagtc
gcacgtcacatttacagattctgtcgaaacttcgctctttaatgtaattcgattccaggtctgaacaaaacatttgtacaaagtaggg
aattctgttgaaatgtg
The LU61965240 cDNA is translated into the following amino acid sequence (SEQ ID NO: 58):
mnteydylfkllligdsgvgksclllrfaddsyldsyistigvdfkirtveqdgktiklqiwdtagqerfrtitssyyrgahgiivvydvtdq
esfnnvkqwlneidryasehvnkllvgnksdltsnkvvsyetgkaladelgipfmetsaknasnvedafmamsaaiktrmas
qptnnakpptvqirgepvnqksgccss
cDNA sequence of LU62294414 from linseed (SEQ ID NO: 59):
ccgaaattgaccccgttctgtttgtgagatctttttgatcattattagccagacagaaacggtgcattaacagttgttgagaggaaa
agcaaagcaaaagcaggaacaagaggaagaagcaagagagaaagaaagcttgcttcttttttttctgttttctgttccatttggg
tggctgctgctggaatttgggaggagaaatttagttctggaatgggatcttcttcaggtagtagtgggtatgatctgtcgttcaagttg
ttgttgattggagattcaagtgttggcaaaagcagcctgcttgtcagcttcatctccaccacctctgctgaagaagatcttgctccca
ccattggtgtggacttcaagatcaagcagctgacagtagctggcaagagattgaagctcaccatttgggatactgctgggcag
gagaggttcaggacactaacaagctcttactacaggaatgcacagggtatcatacttgtttatgacgtgaccaggagagagac
ctttacgaacctatcggacgtatgggctaaagaagttgagctctactgcacaaaccaggactgtgtcaagatgcttgttggcaac
aaagttgacaaagactctgacagaactgtaaccagagaagaaggaatggaacttgcaaaagagcgtggatgtttgttcctcg
agtgcagtgccaaaactcgtgaaaacgtggagcaatgcttcgaggagcttgcgcaaaagataaaggatgttccaagtctcttg
gaagaaggatctacggccgggaagaggaacattctaaagcaaaacccagatcgccaaatgtctcaaagcaacggctgttg
ctcttaaataatgattgactaactgattgatgtatattcagcttcagttctttacctttgtttcttctgtttgtgatttcgagggtgtgtatttccc
agagtttccgattagtttgttgcaaaagattggtttgatgaggctaacggtgaatccagtcgagtcgtcaatgaacgaatgtgatat
gatatatataggtttgtaattgatgt
The LU62294414 cDNA is translated into the following amino acid sequence (SEQ ID NO: 60):
mgsssgssgydlsfkllligdssvgkssllvsfisttsaeedlaptigvdfkikqltvagkrlkltiwdtagqerfrtltssyyrnaqgiilv
ydvtrretftnlsdvwakevelyctnqdcvkmlvgnkvdkdsdrtvtreegmelakergclflecsaktrenveqcfeelaqkikd
vpslleegstagkrnilkqnpdrqmsqsngccs
cDNA sequence of LU61723544 from linseed (SEQ ID NO: 61):
ggtacctgaagaagaaggcctttccctcttcattctgcattttcttttcctctttggcttttccattagatcttcctcttctgcttcttcctgatct
ggttttcctctggaattttctgatttagagagtaaatttgttagcgtttgaatcaatggctgctccgcccgcaagagctcgtgccgatta
tgattaccttataaagctcctcctgatcggcgatagcggtgtgggtaagagttgcctcctcctacgtttctcagatggttccttcacca
ctagtttcattacgaccattggtattgatttcaagataaggacaattgagcttgatggaaaacggatcaagttgcaaatatgggat
actgctggtcaagagcgtttccgcactattacaactgcttactatcgtggagcaatgggtattttgctcgtgtatgatgtcactgatga
gtcatcattcaacaatatcaggaattggattcgcaacattgaacaacatgcctctgataatgtgaacaagatcttggttggaaac
aaagccgatatggatgagagcaaaagggcggttcctaccgcaaagggccaggctcttgcagacgaatacggcatcaagttc
tttgagacgagtgcaaagacaaacttaaacgtggaggaggttttcttctcaatagccagagacatcaagcaacgacttgcaga
tacggattcaaagtccgagccacagacgatcaagattaaccagccggaccaggcgggtggttcgaaccaggctgcacaaa
agtctgcttgctgtggttcttagagattaagacagaaggaataagagtaatatccaattcccttttggccttgtgcgaaattcaaact
cgatactattcgtcttctccctcttcaatctcgtctccacgttttcttcgtcattcttgtttcgcttaattttcgtatgaggttagcgcgacaaa
gagggctgcgattgtttcaccccttctgaaccttaatgtttttgttgcttccttcc
The LU61723544 cDNA is translated into the following amino acid sequence (SEQ ID NO: 62):
maappararadydylikllligdsgvgksclllrfsdgsfttsfittigidfkirtieldgkriklqiwdtagqerfrtittayyrgamgillvyd
vtdessfnnirnwirnieqhasdnvnkilvgnkadmdeskravptakgqaladeygikffetsaktnlnveevffsiardikqrla
dtdsksepqtikinqpdqaggsnqaaqksaccgs
cDNA sequence of LU61871078 from linseed (SEQ ID NO: 63):
aggaactcaattcccttccatctccagacggaattcattcattgagagcaagaaaccctatcatcttcaatcatgggcaccgaat
acgactatctcttcaagcttctgctaatcggcgactcctccgttggaaaatcttgcctgctgctccgatttgctgatgattcgtacgttg
acagctacatcagtactataggagttgatttcaaaatcagaactgtggagctggatggaaagacggtcaagcttcagatctggg
atactgctggtcaggagcgctttagaacaataacaagcagttattaccgaggggcacatggaatcatcattgtctatgatgttact
gacatggacagcttcaacaatgtcaaacaatggttaaatgagattgaccgatatgcaaatgatactgtatgcaagcttttggttgg
gaacaaatgcgatcttgttgagaacaaagttgtcgatacgcagacagcaaaggcgttggccgatgagctaggcatcccttttct
ggagaccagtgccaaagattcaataaatgtggaacaagctttcttaacaatggctgcagaaattaagaaaaaaatgggtaat
caaccgacagctagcaaggcgaccggaacggttcagatgaaaggacaaccgatccagcaaagcaacaactgctgtggtt
aaacctagtcgggctattttgatgtcctgggataagactagtgtggtgaaagtttgtttccatggtttctaggttttctaacttgatgaag
tttagagcaaggtgtagtagattcagttccagataatgtatctccttataatgcttgtaatctatgtgaactgcgatccaatcgagtcg
ttatccgagtagatctcaactgttgtccgttccccagaattcaactggtttaaaatgttgcctttctgc
The LU61871078 cDNA is translated into the following amino acid sequence (SEQ ID NO: 64):
mgteydylfkllligdssvgksclllrfaddsyvdsyistigvdfkirtveldgktvklqiwdtagqerfrtitssyyrgahgiiivydvtd
mdsfnnvkqwlneidryandtvckllvgnkcdlvenkvvdtqtakaladelgipfletsakdsinveqafltmaaeikkkmgnq
ptaskatgtvqmkgqpiqqsnnccg
cDNA sequence of LU61569070 from linseed (SEQ ID NO: 65):
tgaaactctctctctctctctctctctctctctctctctctctctcgtcttcaacaacaacagaaaacatcgccgctgttcgcttcacatct
actccggcgtagctcgatctacgacggttttaggtttcgcttccttctccacgcgttcgtcagctcgccatcatgaactctgagtacg
attacttgttcaagcttttgcttatcggagattccggagtcggcaagtcatgtctacttttgcgattcgctgatgattcgtacttggacag
ttacatcagtaccatcggagtggacttcaaaattcgcaccgtggagcaggatggcaaaaccattaagctccaaatctgggata
cggcagggcaagaacgattcaggaccattacaagtagttactatcgtggtgctcatgggattattgtggtctatgatgtcacaga
ccaagagagtttcaacaatgtcaaacagtggttgagtgaaattgatcgctacgcaagtgagaacgtgaacaaacttctagttgg
gaacaagagtgacctcactgccaacaaagttgtttcatatgaaactgctaaggcatttgccgatgaaattgggattcccttcatgg
agacgagtgccaagaacgcttccaatgtcgaagatgcttttatggcaatgtcagctgcaatcaagaccaggatggctagccaa
cctgtgtcaggcactgccagacctccaacggtgcaaatccgcggagaaccagtgaaccagaagtcaggttgctgctcttcttg
aaaagtagaagcggtggtagtggtgttgggtctctgaagcttaattgtgtgtcctttattatgaatgacatgtaaaactagttctcact
gttgttactgcttttgatgtgaaaaaggatttatttgcatcttttctatttcttgggtcagtttcagtaatgtgttgaaactttgattgttttaaat
gtaatttggtttcaggacaacatttgtacaaattagaaatactgttttgttgaacgcc
The LU61569070 cDNA is translated into the following amino acid sequence (SEQ ID NO: 66):
mnseydylfkllligdsgvgksclllrfaddsyldsyistigvdfkirtveqdgktiklqiwdtagqerfrtitssyyrgahgiivvydvtd
qesfnnvkqwlseidryasenvnkllvgnksdltankvvsyetakafadeigipfmetsaknasnvedafmamsaaiktrma
sqpvsgtarpptvqirgepvnqksgccss
cDNA sequence of OS34999273 from rice (SEQ ID NO: 67):
ttttcccttccgttggtgccattcgtgcagcaccggatcctctcatttctccggcgataactctcccttttccggcgaattcaccgcttcct
cgatatgaatcccgagtatcactatctgttcaagctccttctgattggagactctggtgttggtaaatcatgccttcttctaagatttgct
gatgattcatacattgagagctacataagcaccatcggagttgattttaaaattcgcactgttgagcaggatgggaagacaatta
aactacagatttgggatactgctggacaagaacgatttaggacaataactagtagctactatcgtggagcacatggaatcattat
tgtttatgacgtgacagatgaagatagcttcaataatgtgaagcaatggctcagtgaaattgaccgctatgccagtgataatgtta
acaaacttttggttggaaacaagagtgatctgacagcaaatagagttgtctcatatgacacagctaaggaattcgcagatcaaa
ttggcatacctttcatggaaacaagtgcaaaagatgctacaaatgtggaagatgctttcatggccatgtctgctgccatcaagaat
agaatggctagtcagccttcagcaaacaatgcaaggcctccaacagtgcagatcagagggcaacctgttggacaaaaaagt
ggttgctgctcttcctaaccaggtggtgctgcttggtctacacttaccttttgcatgtaaggggcatatgctatttcactaaatagtgga
ccagtgtcacgtaatccaacctgtggtttgggaattggcctagatgatcccattctttaccatatacttgaatgctatgattgtgcttag
tacttgttaatgataaaacttttatatttctgctc
The OS34999273 cDNA is translated into the following amino acid sequence (SEQ ID NO: 68):
mnpeyhylfkllligdsgvgksclllrfaddsyiesyistigvdfkirtveqdgktiklqiwdtagqerfrtitssyyrgahgiiivydvtde
dsfnnvkqwlseidryasdnvnkllvgnksdltanrvvsydtakefadqigipfmetsakdatnvedafmamsaaiknrmas
qpsannarpptvqirgqpvgqksgccss
cDNA sequence of HA66779896 from sunflower (SEQ ID NO: 69):
gccacctgcaacaaaatctccacaaatctttcactcaaccgatcacaactccacacacaaacaaagatgaatcccgaatacg
actatctgttcaagcttttactcattggagattcaggagttggaaaatcatgtctcctattgcgttttgctgatgattcgtacttggaaagt
tacattagcaccattggggttgactttaaaattcgcactgtggaacaagatggcaaaacaattaagcttcaaatttgggatacag
ctggacaagaacgtttcaggaccatcactagcagctactatcgtggagctcatggcattattgttgtttatgacgtgacagatcaa
gagagtttcaacaacgtgaaacaatggttgagtgaaatcgatcgttacgctagtgagaacgtaaacaagcttcttgtcggaaac
aaatgcgatcttacgtctcagaaagctgtttcctacgaaacaggaaaggcgtttgctgatgagatcgggatcccgtttctcgaaa
caagtgccaagaattccaccaatgtcgaagaggcgtttatggctatgactgctgaaataaaaaacaggatggcaagccagc
cggcaatgaacaatgctagaccgctaactgttgaaatccgaggtcaaccggtcaaccaaaagtcaggatgctgctcttcttga
agagggtaaggatgtgggtggtcaacgtgtgttaagatatgcatttttgttcactcatacttgtcgatgtgaagaagccatttcgttg
atcgccaaacttttgtcattcttttcgatgaattcggggaccttttgtacaaagtaggataagactgttgaatgtgtattatgttatactgt
tttgctgtttgcatttcctttacattttaatgacatttcaagtgtgt
The HA66779896 cDNA is translated into the following amino acid sequence (SEQ ID NO: 70):
mnpeydylfkllligdsgvgksclllrfaddsylesyistigvdfkirtveqdgktiklqiwdtagqerfrtitssyyrgahgiivvydvtd
qesfnnvkqwlseidryasenvnkllvgnkcdltsqkavsyetgkafadeigipfletsaknstnveeafmamtaeiknrmas
qpamnnarpltveirgqpvnqksgccss
cDNA sequence of OS32667913 from rice (SEQ ID NO: 71):
ctcaccaccttcttgttcctggagaacctcctctccagctctgtccaagcatcaattctctttcttttgcttcctgctgatacctttgatcctg
agcagaagaagctgcagaagtgggttaaggcaggaagagccatgaacaacgaatttgattacctgttcaagctgctcctcat
cggcgactcctcggtcggcaagtcatgcttcctcctccgattcgcggacgactcctacgtcgacagctacatcagcacgatcgg
tgttgacttcaagattcgcacgatcgagatggacgggaagaccatcaagctgcagatctgggacacagcaggacaggagc
gattcagaaccatcaccagtagctactaccggggagctcatgggataattatcgtctatgacattacggatatggagagcttcaa
caatgtgaaggagtggatgagcgagatcgacaagtacgccaatgacagcgtatgcaagcttcttgttggtaacaagtgtgatct
ggcagagagcagagttgttgaaactgcagtagcacaggcttatgctgatgagataggcattccattccttgaaacaagtgctaa
ggactcgatcaatgtcgaagaggctttcttggctatgtgtgccgcaatcaaaaagcaaaaatctgggagccaggcagccctgg
agaggaaggcatccaatctagttcagatgaaaggtcagccaattcagcaacagcagcagccacagaagagcagctgttgtt
catcgtgatggcacaatggtctggcatcttccatgaattgggatgaacatggcatatctgttaagtgtgttcctctgtcttctcatagat
ttgagcactttagttactgcaaggtgtcgccacatctgttgaaaatcgagtcaagaacctaatttcctgtctttgatgattctctaataa
acattgcatctagaaagttgtaccatatttaatagatacatgtagtttccagtctgaaaggtcg
The OS32667913 cDNA is translated into the following amino acid sequence (SEQ ID NO: 72):
mnnefdylfkllligdssvgkscfllrfaddsyvdsyistigvdfkirtiemdgktiklqiwdtagqerfrtitssyyrgahgiiivyditdm
esfnnvkewmseidkyandsvckllvgnkcdlaesrvvetavaqayadeigipfletsakdsinveeaflamcaaikkqksg
sqaalerkasnlvqmkgqpiqqqqqpqkssccss
cDNA sequence of HA66453181 from sunflower (SEQ ID NO: 73):
tgtcccccaattctctctctctctctctctctcatcggagcttcaccaccgccggtgatccacaacattcgctatatacctttctccgatc
actatcaacagccatgactcctgagtatgactacctgttcaagcttttgctcattggagattcgggtgtaggaaagtcatgtctactt
ctgaggtttgctgacgattcttacttggacagttacataagcaccatcggagtcgattttaaaattcgtaccgtggagcaagatgcc
aaggttatcaagcttcaaatttgggatactgctggccaagaacgttttaggacaatcacaagcagctactatcgaggagcacat
ggcatcatcgtggtttatgatgtgacggaccaagagagctttaataacgttaagcagtggctgagtgaaatcgaccgttacgcta
gtgagaacgttaacaagatccttgttggaaacaaatgcgatcttgttgcaaataaagtcgtttcaaccgaaacagccaaggcat
ttgctgatgaaattggaattccgttcttggaaacaagtgcaaaagatgcaaccaatgtcgaacagggtcaaccggtctcccaga
acagcggatgctgctcttagtggttgtatttgatgggggtgatgtggcggtgtacaagtattgtccttgtgttactttcatggccatgac
ggcttccatcaaagacaggatggcgagtcaacccaatttgaatacctcaaagcctccaacggtcaacattcgtggggttggatt
ctttttactttctttgtttcagattgtttgcattgtataaaattcaagaattcttttt
The HA66453181 cDNA is translated into the following amino acid sequence (SEQ ID NO: 74):
mtpeydylfkllligdsgvgksclllrfaddsyldsyistigvdfkirtveqdakviklqiwdtagqerfrtitssyyrgahgiivvydvtd
qesfnnvkqwlseidryasenvnkilvgnkcdlvankvvstetakafadeigipfletsakdatnveqafmamtasikdrmas
qpnlntskpptvnirgqpvsqnsgccs
cDNA sequence of HA66709897 from sunflower (SEQ ID NO: 75):
agaaaccaatcatccaccgacaccgtcacaatgagcaacgaatacgattatctcttcaaacttttactcatcggtgactcctccgt
cggaaaatcatgccttcttctccgatttgctgatgattcttatgtggatagttacataagcacaattggagttgactttaaaattagga
ctgtggagcaggataggaagaccatcaagctgcagatatgggatactgctggccaggagcggtttcggactataacaagca
gttactacagaggagcacatggaataattatcgtgtatgatgtgactgagatggagagcttcaacaatgtgaagcaatggctga
gtgaaatcgacagatatgcaaatgaatcagtctgcaagcttcttgttggaaacaaatgtgatctagttgagaacaaggttgttga
cacacaaacagctaaggcatttgcagatgagctcgggatccctttcctcgagaccagtgcaaaagactccgtaaacgtggaa
caggctttcttgacaatggctgcagagataaagaaaaaaatgggtaaccagccaacgggcgacaagagcatagttcaaatc
aaagggcagccgattgagcagaagagcaattgttgtggttaatactgttaaggtccgcaggacaactggtaaaaatgtttgtaa
aatgttgttggcttttaattagcttcatggacttttttgtatcatctgatttcaactacgggtaattttctgcatcaaattactttgaaaggtg
gcaaaatgagcatggttgtgtgacgggtcacaacaggttaaaaaggtcgggccgccgacttgaaacgcttttgatctagttttcg
ttctattacactttgaaatactatcccaataattttttttggattaattagattataagcttacattgctcgacgttggtttatatc
The HA66709897 cDNA is translated into the following amino acid sequence (SEQ ID NO: 76):
msneydylfkllligdssvgksclllrfaddsyvdsyistigvdfkirtveqdrktiklqiwdtagqerfrtitssyyrgahgiiivydvte
mesfnnvkqwlseidryanesvckllvgnkcdlvenkvvdtqtakafadelgipfletsakdsvnveqafltmaaeikkkmgn
qptgdksivqikgqpieqksnccg
TheThe EST443 amino acid sequence (SEQ ID NO: 77):
mvmrkvgkyevgrtigegtfakvkfaqntetgesvamkvldrqtvlkhkmveqirreisimklvrhpnvvrlhevlasrckiyiile
fvtggelfdkivhqgrlnendsrkyfqqlmdgvdychskgvshrdlkpenllldsldnlkisdfglsalpqqvredgllhttcgtpny
vapevlndkgydgavadiwscgvilfvlmagflpfdeadlntlyskireadftcppwfssgaktlitnildpnpltrirmrgirddewf
kknyvpvrmyddedinlddvetafddskeqfvkeqrevkdvgpslmnafelislsqglnlsalfdrrqdhvkrqtrftskkpardii
nrmetaaksmgfgvgtrnykmrleaasecrisqhlavaievyevapslfmievrkaagdtleyhkfyksfctrlkdiiwttavdkd
evktltpsvvknk
The ABJ91230 amino acid sequence (SEQ ID NO: 78):
msssrsggsgsrtrvgryelgrtlgegtfakvkfarnvetgenvaikildkekvlkhkmigqikreistmklirhpnvvrmyevma
sktkiyivlefvtggelfdkiaskgrlkedearkyfqqlinavdychsrgvyhrdlkpenllldasgflkvsdfglsalpqqvredgllht
tcgtpnyvapevinnkgydgakadlwscgvilfvlmagylpfeesnlmalykkifkadftcppwfsssakklikrildpnpstritis
elienewfkkgykpptfekanvslddvdsifnesmdsqnlvverreegfigpmapvtmnafelistsqglnlsslfekqmglvkr
etrftskhsaseiiskieaaaaplgfdvkknnfkmklqgekdgrkgrlsvstevfevapslymvevrksdgdtlefhkfyknlstgl
kdivwktideeeeeeaatng
The ABJ91231 amino acid sequence (SEQ ID NO: 79):
msssrsggggggggggsgsktrvgryelgrtlgegnfakvkfarnvetkenvaikildkenvlkhkmigqikreistmklirhpn
vvrmyevmasktkiyivlqfvtggelfdkiaskgrlkedearkyfqqlicavdychsrgvyhrdlkpenllmdangilkvsdfglsa
lpqqvredgllhttcgtpnyvapevinnkgydgakadlwscgvilfvlmagylpfeeanlmalykkifkadftcppwfsssakkli
krildpnpstritiaelienewfkkgykppafeqanvslddvnsifnesvdsrnlvverreegfigpmapvtmnafelistsqglnls
slfekqmglvkresrftskhsaseiiskieaaaaplgfdvkknnfkmklqgdkdgrkgrlsvateifevapslymvevrksggdtl
efhkfyknlstglkdivwktideekeeeeaatng
The NP_001058901 amino acid sequence (SEQ ID NO: 80):
msvsggrtrvgryelgrtlgegtfakvkfarnadsgenvaikildkdkvlkhkmiaqikreistmklirhpnvirmhevmasktkiy
ivmelvtggelfdkiasrgrlkeddarkyfqqlinavdychsrgvyhrdlkpenllldasgtlkvsdfglsalsqqvredgllhttcgtp
nyvapevinnkgydgakadlwscgvilfvlmagylpfedsnlmslykkifkadfscpswfstsakklikkildpnpstritiaelinn
ewfkkgyqpprfetadvnlddinsifnesgdqtqlvverreerpsvmnafelistsqglnlgtlfekqsqgsvkretrfasrlpaneil
skieaaagpmgfnvqkrnyklklqgenpgrkgqlaiatevfevtpslymvelrksngdtlefhkfyhnisnglkdvmwkpessi
iagdeiqhrrsp
The NP_171622 amino acid sequence (SEQ ID NO: 81):
msgsrrkatpasrtrvgnyemgrtlgegsfakvkyakntvtgdqaaikildrekvfrhkmveqlkreistmklikhpnvveiiev
masktkiyivlelvnggelfdkiaqqgrlkedearryfqqlinavdychsrgvyhrdlkpenlildangvlkvsdfglsafsrqvred
gllhtacgtpnyvapevlsdkgydgaaadvwscgvilfvlmagylpfdepnlmtlykrickaefscppwfsqgakrvikrilepn
pitrisiaelledewfkkgykppsfdqddeditiddvdaafsnskeclvtekkekpvsmnafelissssefslenlfekqaqlvkke
trftsqrsaseimskmeetakplgfnvrkdnykikmkgdksgrkgqlsvatevfevapslhvvelrktggdtlefhkfyknfssgl
kdvvwntdaaaeeqkq
The ABJ91219 amino acid sequence (SEQ ID NO: 82):
msvkvpaartrvgkyelgktigegsfakvkvaknvqtgdvvaikildrdqvlrhkmveqlkreistmklikhpnvikifevmaskt
kiyiviefvdggelfdkiakhgrlkedearryfqqlikavdychsrgvfhrdlkpenllldsrgvlkvsdfglsalsqqlrgdgllhtacg
tpnyvapevlrdqgydgtasdvwscgvilyvlmagflpfsesslvvlyrkicradftfpswfssgakklikrildpkpltritvseiled
ewfkkgykppqfeqeedvniddvdavfndskehlvterkvkpvsinafelisktqgfsldnlfgkqagvvkrethiashspanei
msrieeaakplgfnvdkrnykmklkgdksgrkgqlsvatevfevapslhmvelrkiggdtlefhkfyksfssglkdvvwksdqti
eglr
The BAD12177 amino acid sequence (SEQ ID NO: 83):
maestreenvymaklaeqaeryeemvefmekvaktvdveeltveernllsvayknvigarraswriissieqkeesrgnedh
vssikeyrgkieaelskicdgilnlleshlipvastaeskvfylkmkgdyhrylaefktgaerkeaaentllayksaqdialaelapt
hpirlglalnfsvfyyeilnssdracnlakqafddaiaeldtlgeesykdstlimqllrdnltlwtsdstddagdeikeaskresgdgeq
The AAY67798 amino acid sequence (SEQ ID NO: 84):
mlptessreenvymaklaeqaeryeemvefmekvaktvdveeltveernllsvayknvigarraswriissieqkeesrgne
dhvsiikeyrgkieaelskicdgilslleshlipsassaeskvfylkmkgdyhrylaefktaaerkeaaestllayksaqdialadla
pthpirlglalnfsvfyyeilnspdracnlakqafdeaiseldtlgeesykdstlimqllrdnltlwtsditdeagdeikdaskresgeg
qpqq
The BAD12176 amino acid sequence (SEQ ID NO: 85):
maestreenvymaklaeqaeryeemvefmekvaktvdveeltveernllsvayknvigarraswriissieqkeesrgnedh
vssikeyrgkieaelskicdgilnlleshlipvastaeskvfylkmkgdyhrylaefktgaerkeaaentllayksaqdialaelapt
hpirlglalnfsvfyyeilnssdracnlakqafddaiaeldtlgeesykdstlimqllrdnltlwtsdttddagdeikeaskresgegeq
The AAC04811 amino acid sequence (SEQ ID NO: 86):
mspaepsreenvymaklaeqaeryeemvefmekvartvdteeltveernllsvayknvigarraswriissieqkeesrgne
dhvalikdyrgkieaelskicdgilklldshlvpsstaaeskvfylkmkgdyhrylaefksgaerkeaaestllayksaqdialaela
pthpirlglalnfsvfyyeilnspdracnlakqafdeaiseldtlgeesykdstlimqllrdnltlwtsdineeagdeikeaskagegq
The Q9SP07 amino acid sequence (SEQ ID NO: 87):
mspaepsreenvymaklaeqaeryeemvefmekvartvdteeltveernllsvayknvigarraswriissieqkeesrgne
dhvalikdyrgkieaelskicdgilklldshlvpsstapeskvfylkmkgdyhrylaefksgaerkeaaestllayksaqdialaela
pthpirlglalnfsvfyyeilnspdracnlakqafdeaiseldtlgeesykdstlimqllrdnltlwtsdineeagdeikeaskavegq
The EST217 amino acid sequence (SEQ ID NO: 88):
Mstekeresyvymaklaeqaerydemvesmkkvakldveltveernllsvgyknvigarraswrimssieqkeeskgneq
nvkrikdyrhkveeelskicndilsiidghlipssstgestvfyykmkgdyyrylaefktgnerkeaadqslkayqaasstavtdla
pthpirlglalnfsvfyyeilnsperachlakqafdeaiaeldtlseesykdstlimqllrdnltlwtsdlqdeggddqgkgddmrpeeae

1. A transgenic plant transformed with an expression cassette comprising an isolated polynucleotide encoding a CBL-interacting protein kinase having a sequence as set forth in SEQ ID NO:2.

2. A transgenic plant transformed with an expression cassette comprising an isolated polynucleotide encoding a 14-3-3 protein having a sequence as set forth in SEQ ID NO:4.

3. A transgenic plant transformed with an expression cassette comprising an isolated polynucleotide encoding a RING H2 zinc finger protein or a zinc finger, C3HC4 type domain of a RING H2 zinc finger protein.

4. The transgenic plant of claim 3, wherein the RING H2 zinc finger protein comprises a sequence selected from the group consisting of amino acids 1 to 381 of SEQ ID NO:6; amino acids 1 to 199 of SEQ ID NO:8; amino acids 1 to 268 of SEQ ID NO:10; amino acids 1 to 164 of SEQ ID NO:12; amino acids 1 to 320 of SEQ ID NO:14; amino acids 1 to 219 of SEQ ID NO:16 and amino acids 1 to 177 of SEQ ID NO:18.

5. The transgenic plant of claim 3, wherein the zinc finger, C3HC4 domain is selected from the group consisting of amino acids 88 to 129 of SEQ ID NO:6; amino acids 98 to 139 of SEQ ID NO: 8; amino acids 121 to 162 of SEQ ID NO: 10; amino acids 123 to 164 of SEQ ID NO: 12; amino acids 84 to 125 of SEQ ID NO: 14; amino acids 117 to 158 of SEQ ID NO: 16; amino acids 80 to 121 of SEQ ID NO: 18. More preferably, the transgenic plant of this embodiment comprises a polynucleotide encoding a RING H2 zinc finger protein having a sequence comprising amino acids 1 to 381 of SEQ ID NO:6; amino acids 1 to 199 of SEQ ID NO: 8; amino acids 1 to 268 of SEQ ID NO: 10; amino acids 1 to 278 of SEQ ID NO: 12; amino acids 1 to 320 of SEQ ID NO: 14; amino acids 1 to 219 of SEQ ID NO: 16; amino acids 1 to 177 of SEQ ID NO: 18.

6. A transgenic plant transformed with an expression cassette comprising an isolated polynucleotide encoding a GTP binding protein or a Ras family domain of a GTP binding protein.

7. The transgenic plant of claim 6, wherein the GTP binding protein is selected from the group consisting of a GTP binding protein having a sequence comprising amino acids 1 to 216 of SEQ ID NO:20; amino acids 1 to 184 of SEQ ID NO: 22; amino acids 1 to 191 of SEQ ID NO: 24; amino acids 1 to 214 of SEQ ID NO: 26; amino acids 1 to 182 of SEQ ID NO: 28; amino acids 1 to 181 of SEQ ID NO: 30, amino acids 1 to 193 of SEQ ID NO: 32; amino acids 1 to 183 of SEQ ID NO: 34; amino acids 1 to 193 of SEQ ID NO: 36; amino acids 1 to 193 of SEQ ID NO: 38; amino acids 1 to 193 of SEQ ID NO: 40; amino acids 1 to 181 of SEQ ID NO: 42; amino acids 1 to 193 of SEQ ID NO: 44; amino acids 1 to 204 of SEQ ID NO: 46; amino acids 1 to 182 of SEQ ID NO: 48; amino acids 1 to 214 of SEQ ID NO: 50; amino acids 1 to 206 of SEQ ID NO: 52; amino acids 1 to 204 of SEQ ID NO: 54; amino acids 1 to 158 of SEQ ID NO: 56; amino acids 1 to 202 of SEQ ID NO: 58; amino acids 1 to 212 of SEQ ID NO: 60; amino acids 1 to 216 of SEQ ID NO: 62; amino acids 1 to 201 of SEQ ID NO: 64; amino acids 1 to 203 of SEQ ID NO: 66; amino acids 1 to 203 of SEQ ID NO: 68; amino acids 1 to 203 of SEQ ID NO: 70; amino acids 1 to 209 of SEQ ID NO: 72; amino acids 1 to 202 of SEQ ID NO: 74; and amino acids 1 to 199 of SEQ ID NO: 76.

8. The transgenic plant of claim 6, wherein the Ras family domain is selected from the group consisting of a domain having a sequence comprising amino acids 17 to 179 of SEQ ID NO:20; amino acids 21 to 182 of SEQ ID NO: 22; amino acids 19 to 179 of SEQ ID NO: 24; amino acids 17 to 179 of SEQ ID NO: 26; amino acids 19 to 179 of SEQ ID NO: 28; amino acids 19 to 179 of SEQ ID NO: 30; amino aics 22 to 193 of SEQ ID NO: 32; amino acids 19 to 179 of SEQ ID NO: 34; amino acids 22 to 193 of SEQ ID NO: 36; amino acids 22 to 193 of SEQ ID NO: 38; amino acids 22 to 193 of SEQ ID NO: 40; amino acids 19 to 179 of SEQ ID NO: 42; amino acids 22 to 193 of SEQ ID NO: 44; amino acids 10 to 171 of SEQ ID NO: 46; amino acids 19 to 179 of SEQ ID NO: 48; amino acids 17 to 179 of SEQ ID NO: 50; amino acids 10 to 171 of SEQ ID NO: 52; amino acids 11 to 172 of SEQ ID NO: 54; amino acids 1 to 137 of SEQ ID NO: 56; amino acids 10 to 171 of SEQ ID NO: 58; amino acids 15 to 179 of SEQ ID NO: 60; amino aicds 17 to 195 of SEQ ID NO: 62; amino acids 10 to 171 of SEQ ID NO: 64; amino acids 10 to 171 of SEQ ID NO: 66; amino acids 10 to 171 of SEQ ID NO: 68; amino acids 10 to 171 of SEQ ID NO: 70, amino acids 10 to 171 of SEQ ID NO: 72; amino acids 10 to 171 of SEQ ID NO 74; and amino acids 10 to 171 of SEQ ID NO: 76.

9. An isolated polynucleotide having a sequence selected from the group consisting of the polynucleotide sequences set forth in Table 1.

10. An isolated polypeptide having a sequence selected from the group consisting of the polypeptide sequences set forth in Table 1.

11. A method of producing a transgenic plant comprising at least one polynucleotide listed in Table 1, wherein expression of the polynucleotide in the plant results in the plant's increased growth and/or yield under normal or water-limited conditions and/or increased tolerance to an environmental stress as compared to a wild type variety of the plant comprising the steps of:
(a) introducing into a plant cell an expression vector comprising at least one polynucleotide listed in Table 1, and
(b) generating from the plant cell a transgenic plant that expresses the polynucleotide,
wherein expression of the polynucleotide in the transgenic plant results in the plant's increased growth and/or yield under normal or water-limited conditions and/or increased tolerance to environmental stress as compared to a wild type variety of the plant.

12. A method of increasing a plant's growth and/or yield under normal or water-limited conditions and/or increasing a plant's tolerance to an environmental stress comprising the steps of increasing the expression of at least one polynucleotide listed in Table 1 in the plant.
